(19) United States
(12) Patent Application Publication
(10) Pub. No.: US 2018/0268937 A1
(43) Pub. Date: Sep. 20, 2018

(54) METHOD, APPARATUS, AND COMPUTER PROGRAM PRODUCT FOR ANALYZING BIOLOGICAL DATA

(71) Applicant: Caris Science, Inc., Irving, TX (US)

(72) Inventors: David SPETZLER, Paradise Valley, AZ (US); Quanyuan HE, Chandler, AZ (US); Nianqing XIAO, Rockville, MD (US)

(21) Appl. No.: 15/762,932

(22) PCT Filed: Sep. 24, 2016
(86) PCT No.: PCT/US16/53614

§ 371 Date: Mar. 23, 2018

Related U.S. Application Data
(60) Provisional application No. 62/232,345, filed on Sep. 24, 2015.
(60) Provisional application No. 62/399,376, filed on Sep. 24, 2016.

(51) Int. Cl. G16H 50/20; G06F 3/0484; G16H 50/70; G16H 10/40

(52) U.S. Cl. CPC G16H 50/20; G06F 3/0484; G06T 11/60; G16H 10/40; G16H 50/70

(57) ABSTRACT

A method, apparatus, and computer program product for analyzing biological data with an application that provides a graphical user interface are disclosed. A method in accordance with the disclosure may include receiving patient data associated with various patient attributes, determining an interrelationship between any one of those attributes, performing a therapeutic regime analysis, and displaying at least one graphical user interface. The method may further include assisting in providing patient care based on the interrelationships displayed on the graphical user interface. Patient attributes may include biomarker status that corresponds with the expression, sequence, or other state, of various biomarkers. The method, apparatus, and computer program product are useful for analyzing molecular profiling data.

### CROSS-REFERENCE

This application claims the benefit of priority to United States Provisional Patent Application Ser. No. 62/232,345, filed on Sep. 24, 2015, and 62/399,376, filed on Sep. 24, 2016; both of which applications are incorporated by reference herein in their entirety.

### BACKGROUND OF THE DISCLOSURE

Biomarkers for conditions and diseases such as cancer include biological molecules such as proteins, peptides, lipids, nucleic acids (e.g., DNA, RNA) and variations and modifications thereof. The identification of states of specific biomarkers, such as specific DNA, RNA and proteins, within a biological sample from a patient may provide for a diagnosis, prognosis, and/or theranosis of conditions and/or diseases for the patient. Accordingly, analysis of biomarkers present within a biological sample can assist in the detection of a condition and/or disease, determining the severity of the condition and/or disease, determining predisposition to the condition and/or disease, and/or determine appropriate treatment options.

There remains a need to easily identify biomarkers for detecting and/or treating a condition or disease. In this regard, advancements in computing technology, including increased memory and processing power, as well as advancements in user interface technology, have allowed for application developers to create more complex applications that provide a variety of control features enabling user control of application functionality. Accordingly, the present invention provides systems, apparatus, methods, and computer program products for analyzing biological data such that an analysis of biomarkers may assist in patient care, e.g., by providing for a diagnosis, prognosis, and/or theranosis of conditions and/or diseases present in the patient, or by generating hypotheses for research studies.

### BRIEF SUMMARY OF THE DISCLOSURE

The present disclosure provides systems, methods, apparatuses, and computer program products for providing a user interface for an application for analyzing biological data.

In an aspect, the invention provides a method of analyzing biological data, the method comprising: receiving, at a computing device comprising a processor and memory, patient data for a plurality of patients, the patient data corresponding to at least one of a biological sampling event, a biological processing event, at least one therapeutic regime, at least one biomarker status, and a patient status; determining at least one interrelationship between any one of the biological sampling event, the biological processing event, the at least one therapeutic regime, the at least one biomarker status, and the patient status; performing a therapeutic regime analysis to determine an interrelationship status for the interrelationship between at least one therapeutic regime and at least one of the patient status and the at least one biomarker status; and displaying at least one graphical interface on a user interface in communication with the computing device, the graphical interface including a plurality of visual elements, each visual element of the plurality of visual elements being associated with the patient data, at least one visual element being associated with the at least one interrelationship, at least one visual element including an indicium corresponding to at least one of the interrelationship status and the biomarker status. In a related aspect, the invention provides a method of analyzing biological data associated with a biological sample from a target patient, the method comprising: receiving, at a computing device comprising a processor and memory, patient data associated with the target patient, the patient data corresponding to a biological sampling event, a biological processing event, a therapeutic regime, a marker status, and a patient status; receiving reference data associated with a plurality of patients, the reference data corresponding to a plurality of biological sampling events, biological processing events, therapeutic regimes, marker statuses, and patient statuses; determining at least one interrelationship between any one of the biological sampling events, the biological processing events, the therapeutic regimes, the marker statuses, and the patient statuses; performing a therapeutic regime analysis to determine the interrelationship between at least one therapeutic regime and at least one of the at least one patient status and the at least one marker status; displaying at least one graphical user interface, the graphical user interface configured to: i) display a plurality of graphical user interface objects associated with the reference data, ii) display a plurality of graphical user interface objects associated with the patient data, iii) display, on at least one graphical interface on a user interface in communication with the computing device, a primary graphical user interface object configured to, upon receiving an indication of a user input defining a selection of the primary graphical user interface object, cause the graphical user interface to display a secondary graphical user interface object; and assisting in providing patient care based on the one or more interrelationships displayed on the user interface.

The method of any preceding or subsequent aspect or embodiment, or combinations thereof, may further comprise manipulating a primary visual element to display a secondary visual element including additional information corresponding to the patient data upon selection thereof. The method may further comprise displaying the secondary visual element such that the secondary visual element overlays the primary visual element or the primary visual element is resized such that the secondary visual element is displayed adjacent to the primary visual element.

The method of any preceding or subsequent aspect or embodiment, or combinations thereof, may further comprise assisting in providing patient care based on the one or more interrelationships displayed on the user interface. In some embodiments, assisting in providing the patient care comprises assisting in at least one of providing a diagnosis, providing a prognosis, selecting a recommended therapeutic regime, generating a hypothesis, and evaluating an efficiency of the therapeutic regime, based on the one or more interrelationships. In some embodiments, assisting in providing the patient care comprises selectively manipulating the graphical interface and one or more of the plurality of visual elements displayed thereon to visually compare a target patient against a set of reference patients. Visually comparing the target patient against the set of reference patients can be based on various desired attributes, including without limitation shared patient attributes, the at least one therapeutic regime, and/or the at least one biomarker status.

In the method of any preceding or subsequent aspect or embodiment, or combinations thereof, performing the therapeutic regime analysis may comprise identifying a positive interrelationship status between the at least one therapeutic regime and at least one positive biomarker status in response to determining that the at least one therapeutic regime is likely to be more effective for a condition and/or disease when a positive biomarker status for a particular biomarker is detected in the at least one biological sampling event. The particular biomarker can be a biomarker listed in any one of Tables 1-7. The particular biomarker can be listed elsewhere herein. The particular biomarker may be as described in any one of US Patent Publications US20100113299, published May 6, 2010; US20140222443, published Aug. 7, 2014; US20150307947, published Oct. 29, 2015; US20160186266, published Jun. 30, 2016; and US20150024952, published Jan. 22, 2015; U.S. Pat. No. 8,700,335, issued Apr. 15, 2014 and U.S. Pat. No. 8,768,629, issued Jul. 1, 2014; and Int'l Patent Publications WO2015116868, published Aug. 6, 2015 (equivalent to U.S. patent application Ser. No. 15/115,617, filed Jul. 29, 2016), and WO2016141169, published Sep. 9, 2016; each of which patent publications is incorporated herein by reference in its entirety.

The method of any preceding or subsequent aspect or embodiment, or combinations thereof, may further comprise storing the patient data for the plurality of patients in a clinical database, a biomarker database, a knowledge database, and/or a cohort database comprising a combination of the clinical database, the biomarker database, and the knowledge database. In an embodiment, the method further comprises mapping the patient data from the clinical database, the biomarker database, the knowledge database, and/or the cohort database and storing it in one or more external databases in communication with the computing device. The method may further comprise creating one or more user defined roles to restrict specific users from viewing specific portions of the patient data and/or manipulating the mapped patient data stored in the one or more external databases. The one or more user defined roles can be based on any desired criteria, including without limitation at least one of disease lineage, patient cohort, user affiliation, or user's membership in a study group.

In the method of any preceding or subsequent aspect or embodiment, or combinations thereof, the plurality of visual elements may comprise any useful visual element, including without limitation at least one of a sunburst plot (see, e.g., FIGS. 4N-4Q), a Kaplan Meier plot (see, e.g., FIG. 4B), a waterfall plot (see, e.g., FIGS. 4C-4H), a table (see, e.g., FIG. 4M), a volcano plot (see, e.g., FIGS. 4K-4L), or a graph (see, e.g., FIGS. 4I-4J).

The method of any preceding or subsequent aspect or embodiment, or combinations thereof, may further comprise applying a filter to the patient data to filter the patient data based on any useful attribute, including without limitation at least one of a particular biomarker or group thereof, the at least one biomarker status, a patient cohort, a patient status, the at least one therapeutic regime, the biological processing event, the biological sampling event, at least one indicium listed in Table 1, and any combination thereof. Display of at least one of the plurality of visual elements can be associated with the filtered patient data.

In the method of any preceding or subsequent aspect or embodiment, or combinations thereof, the patient data may further comprise historical data that tracks the patient status over a period of time. In some embodiments, the patient status comprises information associated with an age of the patient, a sex of the patient, a race of the patient, a condition and/or disease of the patient, a status of the condition and/or disease of the patient, and/or an outcome of the condition and/or disease of the patient. The outcome of the condition and/or disease of the patient may comprise any outcome of interest, including without limitation death, partial remission, complete remission, recurrence, or cure.

In the method of any preceding or subsequent aspect or embodiment, or combinations thereof, the condition or disease of the patient may comprise any condition or disease of interest, including without limitation a neoplastic/proliferative disease or disorder, neurological disease or disorder, autoimmune disease or disorder, cardiovascular disease or disorder, or infectious disease. In preferred embodiments, the neoplastic/proliferative disease comprises cancer. The lineage of the cancer can be a lineage listed in Table 1. The lineage of the cancer can be a lineage listed elsewhere herein. In some embodiments, the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumor (GIST), glioblastoma, head and neck squamous carcinoma, leukemia, liver hepatocellular carcinoma, low grade glioma, lung bronchioloalveolar carcinoma (BAC), non-small cell lung cancer (NSCLC), small cell lung cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma. The cancer may be an acute lymphoblastic leukemia; acute myeloid leukemia; adrenocortical carcinoma; AIDS-related cancer; AIDS-related lymphoma; anal cancer; appendix cancer; astrocytomas; atypical teratoid/rhabdoid tumor; basal cell carcinoma; bladder cancer; brain stem glioma; brain tumor, brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma; breast cancer; bronchial tumors; Burkitt lymphoma; cancer of unknown primary site (CUP); carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor. The stage of the cancer may comprise a stage listed in Table 1. For example, the stage can be stage I, stage II, stage III, stage IV, unknown, or various subsets of such stages. In some embodiments, the histology of the cancer is as listed in Table 1.

In the method of any preceding or subsequent aspect or embodiment, or combinations thereof, determining the at least one interrelationship may comprise determining an existence of a relationship between the patient status and the at least one biomarker status based on the biological sampling event.

The method of any preceding or subsequent aspect or embodiment, or combinations thereof, may further comprise determining the at least one biomarker status by detecting at least one biomarker in the at least one biological sampling event and assessing at least one characteristic for the at least one particular biomarker. The at least one characteristic may comprise any desired characteristic, including without limitation at least one of overexpression, underexpression, a modification, a polymorphism, a deletion, an insertion, a substitution, a translocation, a fusion, a break, a duplication, an amplification, a repeat, a copy number variant, a DNA methylation variation, a transcript expression level, a transcript variant, and a splice variant. One of skill will appreciate that the at least one characteristic can be selected based upon the particular biomarker. By way of non-limiting example, the overexpression and underexpression of proteins can be detected using immunological assays and mutations in nucleic acids can be detected via sequence analysis (e.g., Sanger dye-termination sequencing or high throughput next-generation sequencing (NGS)). In come embodiments, detecting the at least one particular biomarker in the at least one biological sampling event comprises assessing a biological sample from a patient using at least one assessment technique. The at least one assessment technique may comprise any useful technique, including without limitation gene expression analysis, nucleic acid sequence analysis, nucleic acid methylation analysis and/or proteomic analysis. The at least one particular biomarker may comprise any useful biomarker, e.g., a protein, a nucleic acid, a lipid, a carbohydrate, or any combination thereof. The at least one particular biomarker can be a biomarker listed in any one of Tables 1-7. The at least one particular biomarker can be listed elsewhere herein. The at least one particular biomarker may be as described in any one of US Patent Publications US20100113299, published May 6, 2010; US20140222443, published Aug. 7, 2014; US20150307947, published Oct. 29, 2015; US20160186266, published Jun. 30, 2016; and US20150024952, published Jan. 22, 2015; U.S. Pat. No. 8,700,335, issued Apr. 15, 2014 and U.S. Pat. No. 8,768,629, issued Jul. 1, 2014; and Int'l Patent Publications WO2015116868, published Aug. 6, 2015, and WO2016141169, published Sep. 9, 2016; each of which patent publications is incorporated herein by reference in its entirety.

The method of any preceding or subsequent aspect or embodiment, or combinations thereof, may further comprise processing the patient data to determine which members of the plurality of patients are matched and which members of the plurality of patients are unmatched. As further described herein, “matched” patient data may originate from patients who had molecular profiling performed and who received one or more therapeutic regime predicted to provide a benefit in treating a condition and/or disease based on the molecular profiling. And “unmatched” patient data may originate from patients who had molecular profiling performed and who received one or more therapeutic regime predicted to provide a potential lack of benefit in treating the condition and/or disease based on the molecular profiling. The patient data can also be processed to determine patients with mixed matched/unmatched treatments or treatments that were neither matched nor unmatched. In some embodiments, the method further comprises performing a survival analysis to compare the unmatched and matched patient data. Such embodiments may further comprise displaying on the at least one graphical user interface a visual element associated with the survival analysis. The visual element associated with the survival analysis can be a Kaplan Meier plot (see, e.g., FIGS. 4A-4C, 4E, 4H, 4K-N) or other appropriate visual element as desired.

In another related aspect, the invention provides a computer-readable storage medium that is non-transitory and has computer-readable program code portions stored therein that, in response to execution by a processor, cause an apparatus to at least: receive, at a computing device comprising the processor and memory, patient data for a plurality of patients, the patient data corresponding to at least one of a biological sampling event, a biological processing event, at least one therapeutic regime, at least one biomarker status, and a patient status; determine at least one interrelationship between any one of the biological sampling event, the biological processing event, the at least one therapeutic regime, the at least one biomarker status, and the patient status; perform a therapeutic regime analysis to determine an interrelationship status for the interrelationship between at least one therapeutic regime and at least one of the patient status and the at least one biomarker status; and display at least one graphical interface on a user interface in communication with the computing device, the graphical interface including a plurality of visual elements, each visual element of the plurality of visual elements being associated with the patient data, at least one visual element being associated with the at least one interrelationship, at least one visual element including an indicium corresponding to at least one of the interrelationship status and the biomarker status.

The computer readable storage medium of any preceding or subsequent aspect or embodiment, or combinations thereof, may have computer-readable program code portions stored therein that cause the apparatus to manipulate a primary visual element to display a secondary visual element including additional information corresponding to the patient data upon selection thereof. The apparatus may be caused to display the secondary visual element such that the secondary visual element overlays the primary visual element or the primary visual element is resized such that the secondary visual element is displayed adjacent to the primary visual element.

The computer readable storage medium of any preceding or subsequent aspect or embodiment, or combinations thereof, may have computer-readable program code portions stored therein that cause the apparatus to assist in providing patient care based on the one or more interrelationships displayed on the user interface. In some embodiments, the apparatus is caused to assist in at least one of providing a diagnosis, providing a prognosis, selecting a recommended therapeutic regime, generating a hypothesis, and evaluating an efficiency of the therapeutic regime, based on the one or more interrelationships in order to assist in providing the patient care.

The computer readable storage medium of any preceding or subsequent aspect or embodiment, or combinations thereof, may have computer-readable program code portions stored therein that cause the apparatus to selectively manipulate the graphical interface and one or more of the plurality of visual elements displayed thereon to visually compare a target patient against a set of reference patients in order to assist in providing the patient care. A visual comparison of the target patient against the set of reference patients can be based on various desired attributes, including without limitation shared patient attributes, the at least one therapeutic regime, and/or the at least one biomarker status.

In the computer-readable program code portions stored within the computer readable storage medium of any preceding or subsequent aspect or embodiment, or combinations thereof, performing the therapeutic regime analysis may comprise identifying a positive interrelationship status between the at least one therapeutic regime and at least one positive biomarker status in response to determining that the at least one therapeutic regime is likely to be more effective for a condition and/or disease when a positive biomarker status for a particular biomarker is detected in the at least one biological sampling event. The particular biomarker can be a biomarker listed in any one of Tables 1-7. The particular biomarker can be listed elsewhere herein. The particular biomarker may be as described in any one of US Patent Publications US20100113299, published May 6, 2010; US20140222443, published Aug. 7, 2014; US20150307947, published Oct. 29, 2015; US20160186266, published Jun. 30, 2016; and US20150024952, published Jan. 22, 2015; U.S. Pat. No. 8,700,335, issued Apr. 15, 2014 and U.S. Pat. No. 8,768,629, issued Jul. 1, 2014; and Int'l Patent Publications WO2015116868, published Aug. 6, 2015, and WO2016141169, published Sep. 9, 2016; each of which patent publications is incorporated herein by reference in its entirety.

The computer readable storage medium of any preceding or subsequent aspect or embodiment, or combinations thereof, may have computer-readable program code portions stored therein that cause the apparatus to store the patient data for the plurality of patients in a clinical database, a biomarker database, a knowledge database, and/or a cohort database comprising a combination of the clinical database, the biomarker database, and the knowledge database. In an embodiment, the apparatus is caused to map the patient data from the clinical database, the biomarker database, the knowledge database, and/or the cohort database and storing it in one or more external databases in communication with the computing device. In some embodiments, the apparatus is caused to create one or more user defined roles to restrict specific users from viewing specific portions of the patient data and/or manipulating the mapped patient data stored in the one or more external databases. The one or more user defined roles can be based on any desired criteria, including without limitation at least one of disease lineage, patient cohort, user affiliation, or user's membership in a study group.

Within the computer-readable program code portions stored within the computer readable storage medium of any preceding or subsequent aspect or embodiment, or combinations thereof, the plurality of visual elements may comprise any useful visual element, including without limitation at least one of a sunburst plot (see, e.g., FIGS. 4N-4Q), a Kaplan Meier plot (see, e.g., FIG. 4B), a waterfall plot (see, e.g., FIGS. 4C-4H), a table (see, e.g., FIG. 4M), a volcano plot (see, e.g., FIGS. 4K-4L), or a graph (see, e.g., FIGS. 4I-4J).

The computer readable storage medium of any preceding or subsequent aspect or embodiment, or combinations thereof, may have computer-readable program code portions stored therein that cause the apparatus to apply a filter to the patient data to filter the patient data based on any useful attribute, including without limitation at least one of a particular biomarker or group thereof, the at least one biomarker status, a patient cohort, a patient status, the at least one therapeutic regime, the biological processing event, the biological sampling event, at least one indicium listed in Table 1, and any combination thereof. Display of at least one of the plurality of visual elements can be associated with the filtered patient data.

The computer readable storage medium of any preceding or subsequent aspect or embodiment, or combinations thereof, may have computer-readable program code portions stored therein that cause the apparatus to process patient data comprising historical data that tracks the patient status over a period of time. In some embodiments, the patient status comprises information associated with an age of the patient, a sex of the patient, a race of the patient, a condition and/or disease of the patient, a status of the condition and/or disease of the patient, and/or an outcome of the condition and/or disease of the patient. The outcome of the condition and/or disease of the patient may comprise any outcome of interest, including without limitation death, partial remission, complete remission, recurrence, or cure. The condition or disease of the patient may comprise any condition or disease of interest, including without limitation a neoplastic/proliferative disease or disorder, neurological disease or disorder, autoimmune disease or disorder, cardiovascular disease or disorder, or infectious disease. In preferred embodiments, the neoplastic/proliferative disease comprises cancer. The lineage of the cancer can be a lineage listed in Table 1. The lineage of the cancer can be a lineage listed elsewhere herein. In some embodiments, the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumor (GIST), glioblastoma, head and neck squamous carcinoma, leukemia, liver hepatocellular carcinoma, low grade glioma, lung bronchioloalveolar carcinoma (BAC), non-small cell lung cancer (NSCLC), small cell lung cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma. The cancer may be an acute lymphoblastic leukemia; acute myeloid leukemia; adrenocortical carcinoma; AIDS-related cancer; AIDS-related lymphoma; anal cancer; appendix cancer; astrocytomas; atypical teratoid/rhabdoid tumor; basal cell carcinoma; bladder cancer; brain stem glioma; brain tumor, brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma; breast cancer; bronchial tumors; Burkitt lymphoma; cancer of unknown primary site (CUP); carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor. The stage of the cancer may comprise a stage listed in Table 1. For example, the stage can be stage I, stage II, stage III, stage IV, unknown, or various subsets of such stages. In some embodiments, the histology of the cancer is as listed in Table 1.

The computer readable storage medium of any preceding or subsequent aspect or embodiment, or combinations thereof, may have computer-readable program code portions stored therein that cause the apparatus to determine an existence of a relationship between the patient status and the at least one biomarker status based on the biological sampling event to determine the at least one interrelationship.

The computer readable storage medium of any preceding or subsequent aspect or embodiment, or combinations thereof, may have computer-readable program code portions stored therein that cause the apparatus to determine the at least one biomarker status by detecting at least one biomarker in the at least one biological sampling event and assessing at least one characteristic for the at least one particular biomarker The at least one characteristic may comprise any desired characteristic, including without limitation at least one of overexpression, underexpression, a modification, a polymorphism, a deletion, an insertion, a substitution, a translocation, a fusion, a break, a duplication, an amplification, a repeat, a copy number variant, a DNA methylation variation, a transcript expression level, a transcript variant, and a splice variant. One of skill will appreciate that the at least one characteristic can be selected based upon the particular biomarker. By way of non-limiting example, the overexpression and underexpression of proteins can be detected using immunological assays and mutations in nucleic acids can be detected via sequence analysis (e.g., Sanger dye-termination sequencing or high throughput next-generation sequencing (NGS)).

The computer readable storage medium of any preceding or subsequent aspect or embodiment, or combinations thereof, may have computer-readable program code portions stored therein that cause the apparatus to assess a biological sample from a patient using data generated via at least one assessment technique to detect the at least one particular biomarker in the at least one biological sampling event. The at least one assessment technique may comprise any useful technique, including without limitation gene expression analysis, nucleic acid sequence analysis, nucleic acid methylation analysis and/or proteomic analysis. The at least one particular biomarker may comprise any useful biomarker, e.g., a protein, a nucleic acid, a lipid, a carbohydrate, or any combination thereof. The at least one particular biomarker can be a biomarker listed in any one of Tables 1-7. The at least one particular biomarker can be listed elsewhere herein. The at least one particular biomarker may be as described in any one of US Patent Publications US20100113299, published May 6, 2010; US20140222443, published Aug. 7, 2014; US20150307947, published Oct. 29, 2015; US20160186266, published Jun. 30, 2016; and US20150024952, published Jan. 22, 2015; U.S. Pat. No. 8,700,335, issued Apr. 15, 2014 and U.S. Pat. No. 8,768,629, issued Jul. 1, 2014; and Int'l Patent Publications WO2015116868, published Aug. 6, 2015, and WO2016141169, published Sep. 9, 2016; each of which patent publications is incorporated herein by reference in its entirety.

The computer readable storage medium of any preceding or subsequent aspect or embodiment, or combinations thereof, may cause the apparatus to process the patient data to determine which members of the plurality of patients are matched and which members of the plurality of patients are unmatched. The patient data can also be processed to determine patients with mixed matched/unmatched treatments or treatments that were neither matched nor unmatched. In some embodiments, the apparatus is caused to perform a survival analysis with the unmatched and matched patient data. The apparatus can further be caused to display on the at least one graphical user interface a visual element associated with the survival analysis. The visual element associated with the survival analysis can be a Kaplan Meier plot (see, e.g., FIGS. 4A-4C, 4E, 4H, 4K-N) or other appropriate visual element as desired.

In still another related aspect, the invention provides an apparatus for analyzing biological data, the apparatus including a user interface, and a computing device in communication with the user interface, the computing device comprising a processor and memory including computer-readable program code stored therein, the computer-readable code configured, upon the execution thereof by the processor, to cause the apparatus to: receive patient data for a plurality of patients, the patient data corresponding to at least one of a biological sampling event, a biological processing event, at least one therapeutic regime, at least one biomarker status, and a patient status; determine at least one interrelationship between any one of the biological sampling event, the biological processing event, the at least one therapeutic regime, the at least one biomarker status, and the patient status; perform a therapeutic regime analysis to determine an interrelationship status for the interrelationship between at least one therapeutic regime and at least one of the patient status and the at least one biomarker status; and display at least one graphical interface on the user interface, the graphical interface including a plurality of visual elements, each visual element of the plurality of visual elements being associated with the patient data, at least one visual element being associated with the at least one interrelationship, at least one visual element including an indicium corresponding to at least one of the interrelationship status and the biomarker status.

The apparatus of any preceding or subsequent aspect or embodiment, or combinations thereof, may be caused to manipulate a primary visual element to display a secondary visual element including additional information corresponding to the patient data upon selection thereof. The apparatus may be caused to display the secondary visual element such that the secondary visual element overlays the primary visual element or the primary visual element is resized such that the secondary visual element is displayed adjacent to the primary visual element.

The apparatus of any preceding or subsequent aspect or embodiment, or combinations thereof, may be caused to assist in providing patient care based on the one or more interrelationships displayed on the user interface. In some embodiments, the apparatus is caused to assist in at least one of providing a diagnosis, providing a prognosis, selecting a recommended therapeutic regime, generating a hypothesis, and evaluating an efficiency of the therapeutic regime, based on the one or more interrelationships in order to assist in providing the patient care.

The apparatus of any preceding or subsequent aspect or embodiment, or combinations thereof, may be caused to selectively manipulate the graphical interface and one or more of the plurality of visual elements displayed thereon to visually compare a target patient against a set of reference patients in order to assist in providing the patient care. A visual comparison of the target patient against the set of reference patients can be based on various desired attributes, including without limitation shared patient attributes, the at least one therapeutic regime, and/or the at least one biomarker status.

In regards to the apparatus of any preceding or subsequent aspect or embodiment, or combinations thereof, performing the therapeutic regime analysis may comprise identifying a positive interrelationship status between the at least one therapeutic regime and at least one positive biomarker status in response to determining that the at least one therapeutic regime is likely to be more effective for a condition and/or disease when a positive biomarker status for a particular biomarker is detected in the at least one biological sampling event. The particular biomarker can be a biomarker listed in any one of Tables 1-7. The particular biomarker can be listed elsewhere herein. The particular biomarker may be as described in any one of US Patent Publications US20100113299, published May 6, 2010; US20140222443, published Aug. 7, 2014; US20150307947, published Oct. 29, 2015; US20160186266, published Jun. 30, 2016; and US20150024952, published Jan. 22, 2015; U.S. Pat. No. 8,700,335, issued Apr. 15, 2014 and U.S. Pat. No. 8,768,629, issued Jul. 1, 2014; and Int'l Patent Publications WO2015116868, published Aug. 6, 2015, and WO2016141169, published Sep. 9, 2016; each of which patent publications is incorporated herein by reference in its entirety.

The apparatus of any preceding or subsequent aspect or embodiment, or combinations thereof, may be caused to store the patient data for the plurality of patients in a clinical database, a biomarker database, a knowledge database, and/or a cohort database comprising a combination of the clinical database, the biomarker database, and the knowledge database. In an embodiment, the apparatus is caused to map the patient data from the clinical database, the biomarker database, the knowledge database, and/or the cohort database and storing it in one or more external databases in communication with the computing device. In some embodiments, the apparatus is caused to create one or more user defined roles to restrict specific users from viewing specific portions of the patient data and/or manipulating the mapped patient data stored in the one or more external databases. The one or more user defined roles can be based on any desired criteria, including without limitation at least one of disease lineage, patient cohort, user affiliation, or user's membership in a study group.

In regards to the apparatus of any preceding or subsequent aspect or embodiment, or combinations thereof, the plurality of visual elements may comprise any useful visual element, including without limitation at least one of a sunburst plot (see, e.g., FIGS. 4N-4Q), a Kaplan Meier plot (see, e.g., FIG. 4B), a waterfall plot (see, e.g., FIGS. 4C-4H), a table (see, e.g., FIG. 4M), a volcano plot (see, e.g., FIGS. 4K-4L), or a graph (see, e.g., FIGS. 4I-4J).

The apparatus of any preceding or subsequent aspect or embodiment, or combinations thereof, may be caused to apply a filter to the patient data to filter the patient data based on any useful attribute, including without limitation at least one of a particular biomarker or group thereof, the at least one biomarker status, a patient cohort, a patient status, the at least one therapeutic regime, the biological processing event, the biological sampling event, at least one indicium listed in Table 1, and any combination thereof. Display of at least one of the plurality of visual elements can be associated with the filtered patient data.

In regards to the apparatus of any preceding or subsequent aspect or embodiment, or combinations thereof, the patient data may comprise historical data that tracks the patient status over a period of time. In some embodiments, the patient status comprises information associated with an age of the patient, a sex of the patient, a race of the patient, a condition and/or disease of the patient, a status of the condition and/or disease of the patient, and/or an outcome of the condition and/or disease of the patient. The outcome of the condition and/or disease of the patient may comprise any outcome of interest, including without limitation death, partial remission, complete remission, recurrence, or cure. The condition or disease of the patient may comprise any condition or disease of interest, including without limitation a neoplastic/proliferative disease or disorder, neurological disease or disorder, autoimmune disease or disorder, cardiovascular disease or disorder, or infectious disease. In preferred embodiments, the neoplastic/proliferative disease comprises cancer. The lineage of the cancer can be a lineage listed in Table 1. The lineage of the cancer can be a lineage listed elsewhere herein. In some embodiments, the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumor (GIST), glioblastoma, head and neck squamous carcinoma, leukemia, liver hepatocellular carcinoma, low grade glioma, lung bronchioloalveolar carcinoma (BAC), non-small cell lung cancer (NSCLC), small cell lung cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma. The cancer may be an acute lymphoblastic leukemia; acute myeloid leukemia; adrenocortical carcinoma; AIDS-related cancer; AIDS-related lymphoma; anal cancer; appendix cancer; astrocytomas; atypical teratoid/rhabdoid tumor; basal cell carcinoma; bladder cancer; brain stem glioma; brain tumor, brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma; breast cancer; bronchial tumors; Burkitt lymphoma; cancer of unknown primary site (CUP); carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor. The stage of the cancer may comprise a stage listed in Table 1. For example, the stage can be stage I, stage II, stage III, stage IV, unknown, or various subsets of such stages. In some embodiments, the histology of the cancer is as listed in Table 1.

The apparatus of any preceding or subsequent aspect or embodiment, or combinations thereof, may be caused to determine an existence of a relationship between the patient status and the at least one biomarker status based on the biological sampling event to determine the at least one interrelationship.

The apparatus of any preceding or subsequent aspect or embodiment, or combinations thereof, may be caused to determine the at least one biomarker status by detecting at least one biomarker in the at least one biological sampling event and assessing at least one characteristic for the at least one particular biomarker. The at least one characteristic may comprise any desired characteristic, including without limitation at least one of overexpression, underexpression, a modification, a polymorphism, a deletion, an insertion, a substitution, a translocation, a fusion, a break, a duplication, an amplification, a repeat, a copy number variant, a DNA methylation variation, a transcript expression level, a transcript variant, and a splice variant. One of skill will appreciate that the at least one characteristic can be selected based upon the particular biomarker. By way of non-limiting example, the overexpression and underexpression of proteins can be detected using immunological assays and mutations in nucleic acids can be detected via sequence analysis (e.g., Sanger dye-termination sequencing or high throughput next-generation sequencing (NGS)).

The apparatus of any preceding or subsequent aspect or embodiment, or combinations thereof, may be caused to assess a biological sample from a patient using at least one assessment technique. The at least one assessment technique may comprise any useful technique, including without limitation gene expression analysis, nucleic acid sequence analysis, nucleic acid methylation analysis and/or proteomic analysis to detect the at least one particular biomarker in the at least one biological sampling event. The at least one particular biomarker may comprise any useful biomarker, e.g., a protein, a nucleic acid, a lipid, a carbohydrate, or any combination thereof. The at least one particular biomarker can be a biomarker listed in any one of Tables 1-7. The at least one particular biomarker can be listed elsewhere herein. The at least one particular biomarker may be as described in any one of US Patent Publications US20100113299, published May 6, 2010; US20140222443, published Aug. 7, 2014; US20150307947, published Oct. 29, 2015; US20160186266, published Jun. 30, 2016; and US20150024952, published Jan. 22, 2015; U.S. Pat. No. 8,700,335, issued Apr. 15, 2014 and U.S. Pat. No. 8,768,629, issued Jul. 1, 2014; and Int'l Patent Publications WO2015116868, published Aug. 6, 2015, and WO2016141169, published Sep. 9, 2016; each of which patent publications is incorporated herein by reference in its entirety.

The apparatus of any preceding or subsequent aspect or embodiment, or combinations thereof, may be caused to process the patient data to determine which members of the plurality of patients are matched and which members of the plurality of patients are unmatched. The patient data can also be processed to determine patients with mixed matched/unmatched treatments or treatments that were neither matched nor unmatched. In some embodiments, the apparatus is caused to perform a survival analysis with the unmatched and matched patient data. The apparatus can further be caused to display on the at least one graphical user interface a visual element associated with the survival analysis. The visual element associated with the survival analysis can be a Kaplan Meier plot (see, e.g., FIGS. 4A-4C, 4E, 4H, 4K-N) or other appropriate visual element as desired.

These and other features, aspects, embodiments, and advantages of the present disclosure will be apparent from a reading of the following detailed description together with the accompanying drawings, which are briefly described below. The present disclosure includes any combination of two, three, four, or more features or elements set forth in this disclosure or recited in any one or more of the claims, regardless of whether such features or elements are expressly combined or otherwise recited in a specific embodiment description or claim herein. This disclosure is intended to be read holistically such that any separable features or elements of the disclosure, in any of its aspects and embodiments, should be viewed as intended, namely to be combinable, unless the context of the disclosure clearly dictates otherwise.

### BRIEF DESCRIPTION OF THE SEVERAL VIEWS OF THE DRAWINGS

Having thus described the disclosure in general terms, reference will now be made to the accompanying drawings, which are not necessarily drawn to scale, and wherein:

FIG. 1 illustrates a block diagram of an apparatus for providing a user interface for an application for analyzing biological data according to one aspect of the present disclosure;

FIG. 2 illustrates an example system that may provide a user interface for an application for analyzing biological data according to one aspect of the present disclosure;

FIG. 3 illustrates a flowchart according to an example method for analyzing biological data according to one aspect of the present disclosure;

FIG. 4A illustrates an example of a display of a user interface for an application for analyzing biological data according to one aspect of the present disclosure;

FIG. 4B illustrates a zoomed view of a portion of FIG. 4A according to one aspect of the present disclosure;

FIG. 4C illustrates an example of a display of a user interface for an application for analyzing biological data according to one aspect of the present disclosure;

FIG. 4D illustrates a zoomed view of the portion of FIG. 4C according to one aspect of the present disclosure;

FIG. 4E illustrates an example of a display of a user interface for an application for analyzing biological data according to one aspect of the present disclosure;

FIG. 4F illustrates a detailed example of a portion of FIG. 4E according to one aspect of the present disclosure;

FIG. 4G an example of a display of a user interface for an application for analyzing biological data according to one aspect of the present disclosure;

FIG. 4H illustrates an example of a display of a user interface for an application for analyzing biological data according to one aspect of the present disclosure;

FIG. 4I illustrates an example of a display of a user interface for an application for analyzing biological data according to one aspect of the present disclosure;

FIG. 4J illustrates a zoomed view of a portion of FIG. 4I according to one aspect of the present disclosure;

FIG. 4K illustrates an example of a display of a user interface for an application for analyzing biological data according to one aspect of the present disclosure;

FIG. 4L illustrates an example of a display of a user interface for an application for analyzing biological data according to one aspect of the present disclosure;

FIG. 4M illustrates an example of a display of a user interface for an application for analyzing biological data according to one aspect of the present disclosure;

FIG. 4N illustrates an example of a display of a user interface for an application for analyzing biological data according to one aspect of the present disclosure;

FIG. 4O illustrates an example of a display of a user interface for an application for analyzing biological data according to one aspect of the present disclosure;

FIG. 4P illustrates an example of a display of a user interface for an application for analyzing biological data according to one aspect of the present disclosure;

FIG. 4Q illustrates an example of a display of a user interface for an application for analyzing biological data according to one aspect of the present disclosure;

FIG. 4R illustrates an example display of a user interface for an application for analyzing biological data according to one aspect of the present disclosure; and

FIG. 5 illustrates an example data storage arrangement for an application for analyzing biological data according to one aspect of the present disclosure.

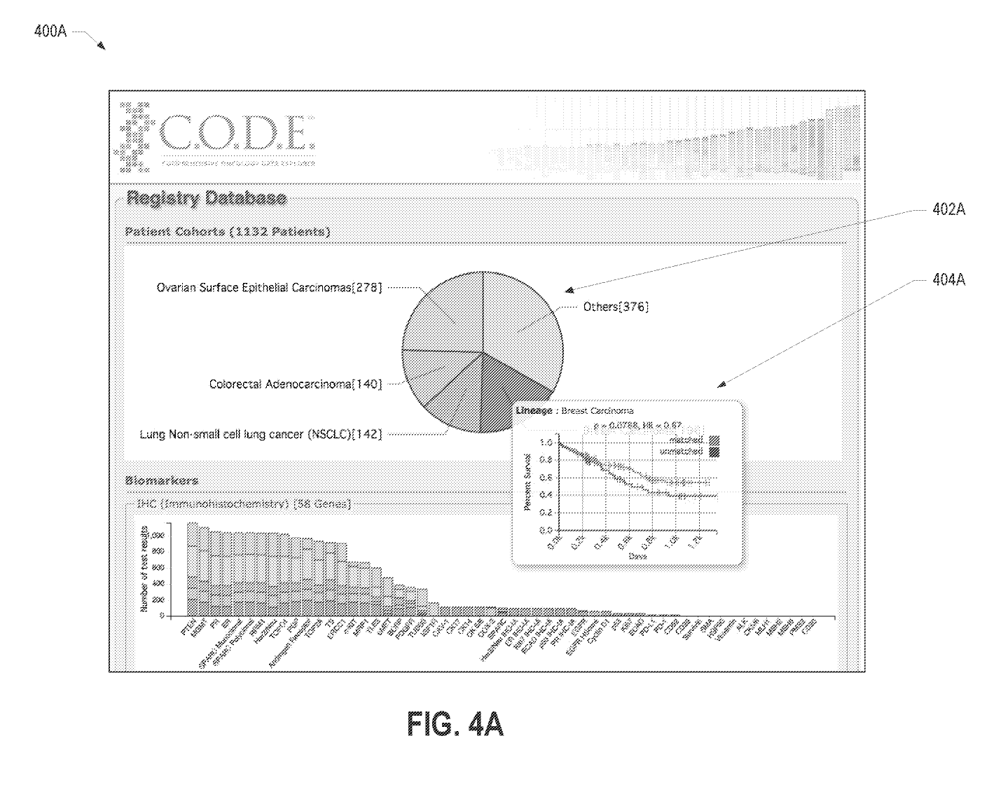

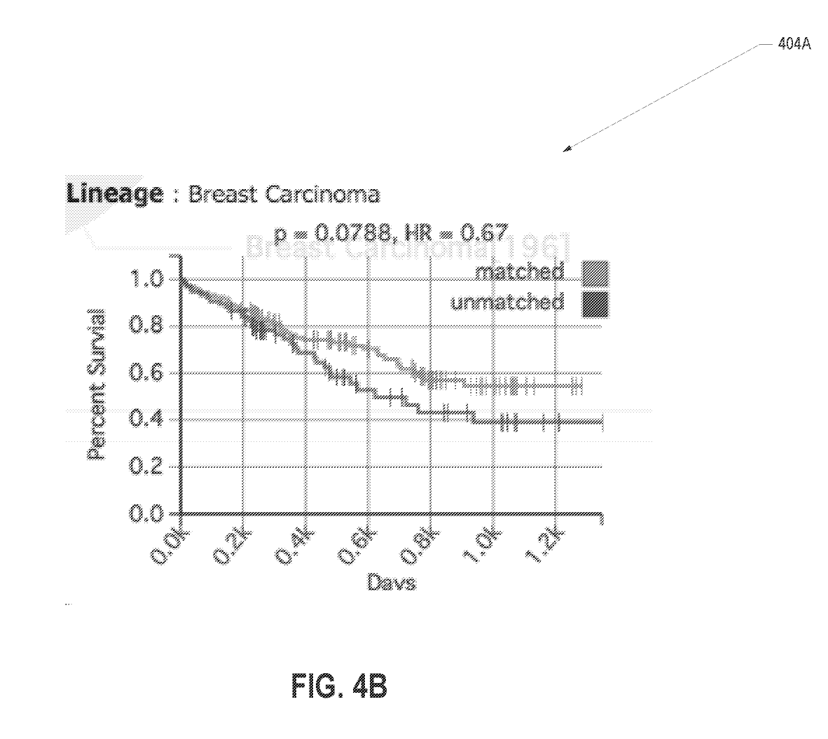

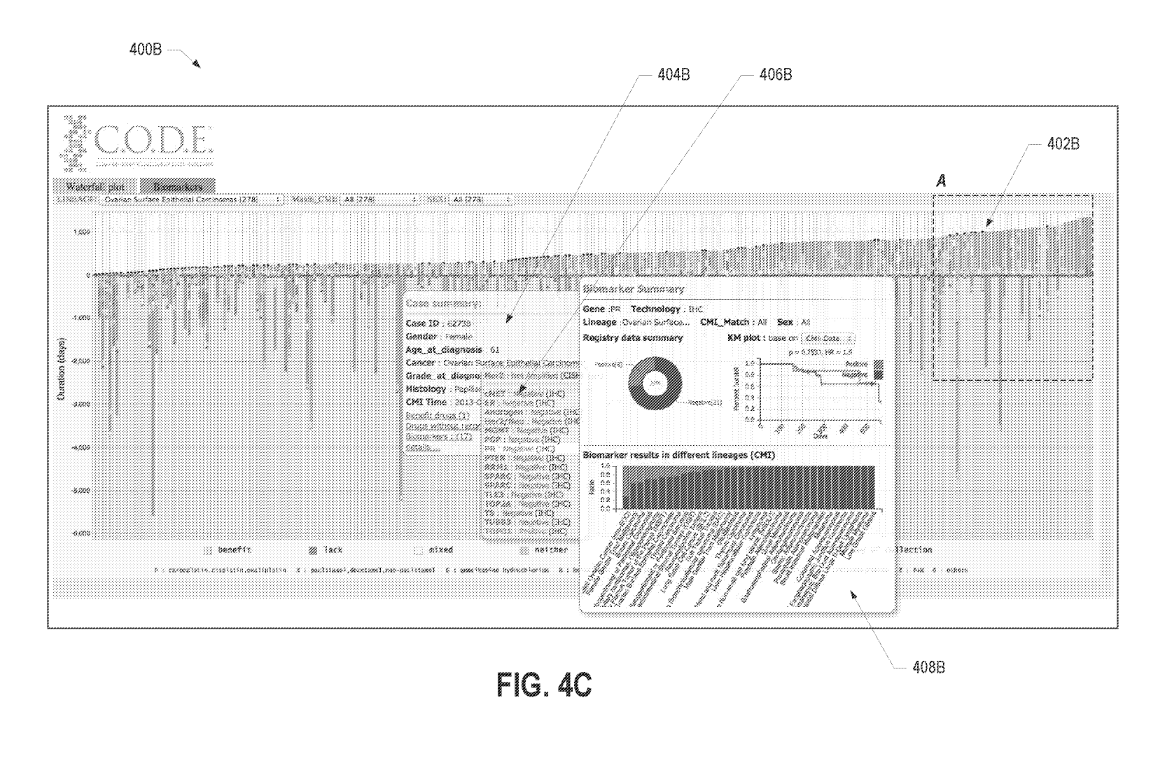

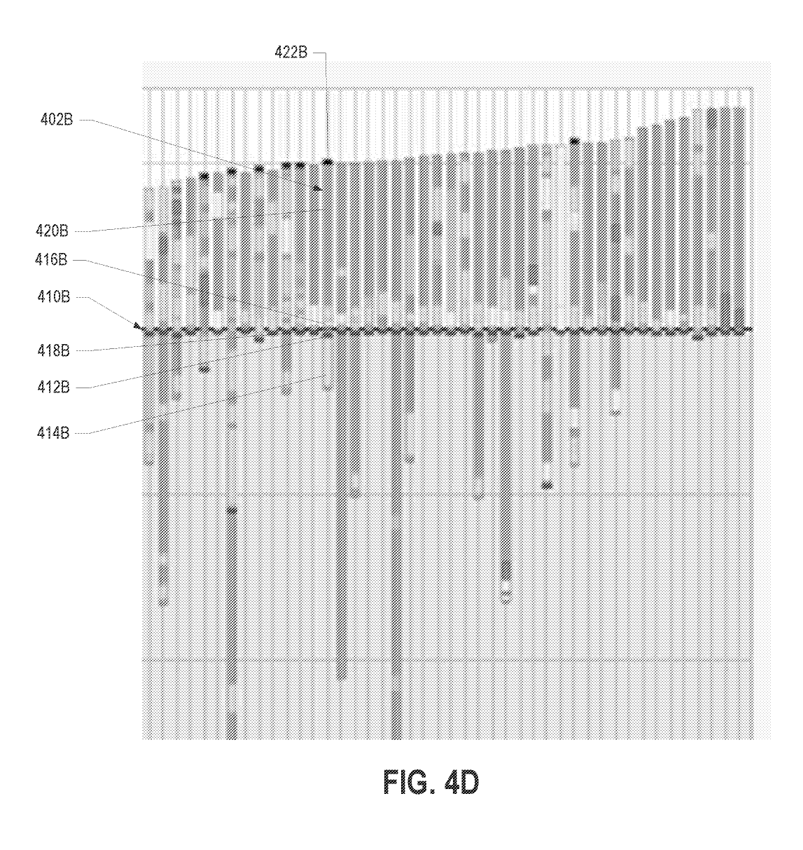

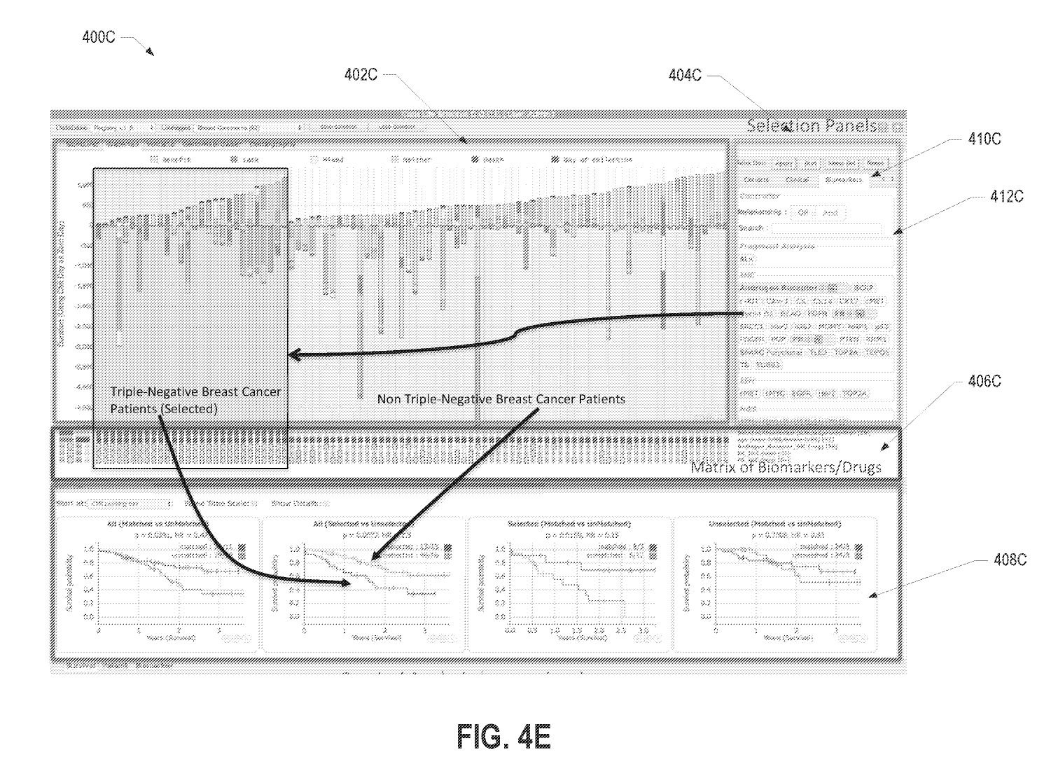

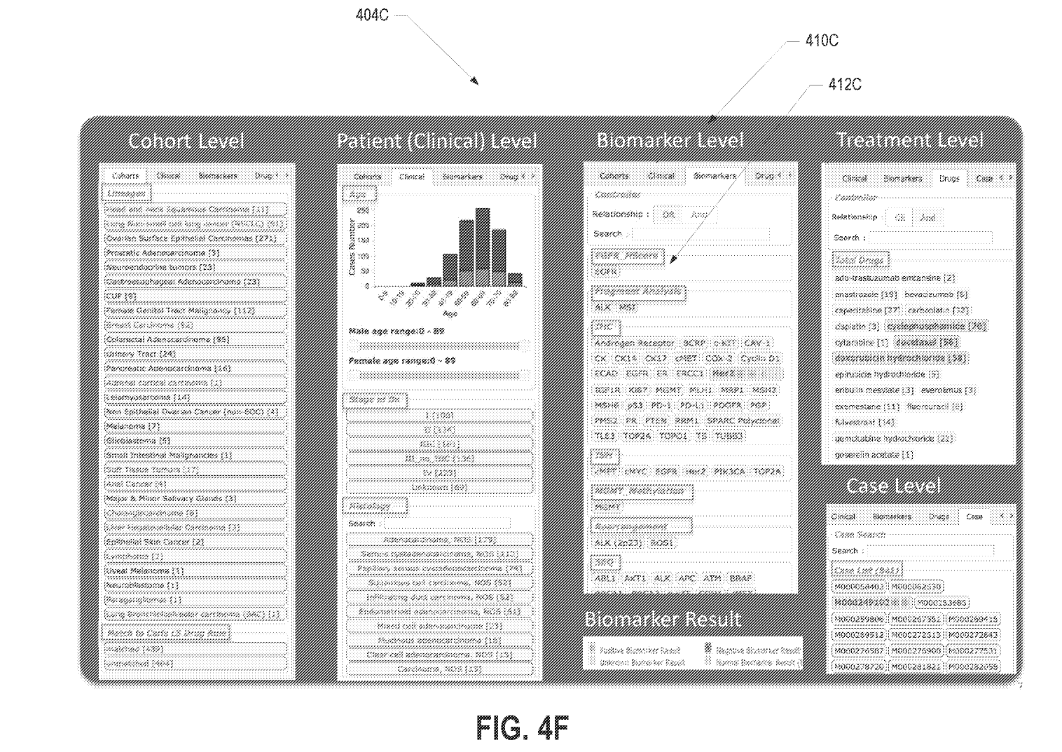

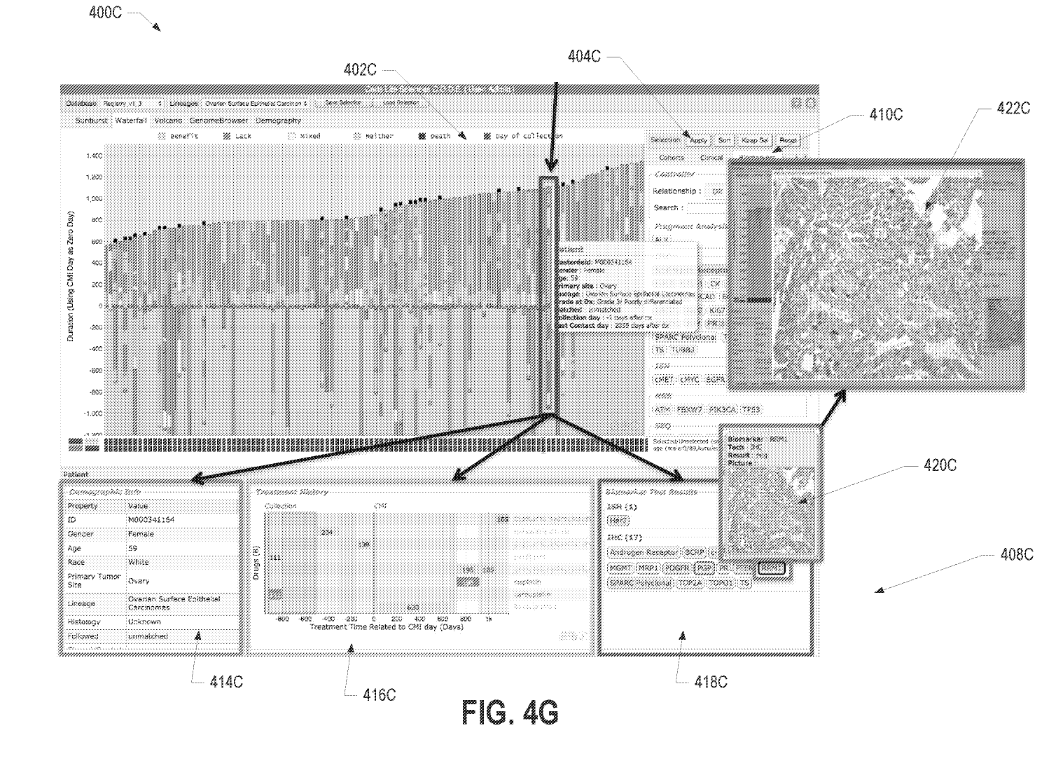

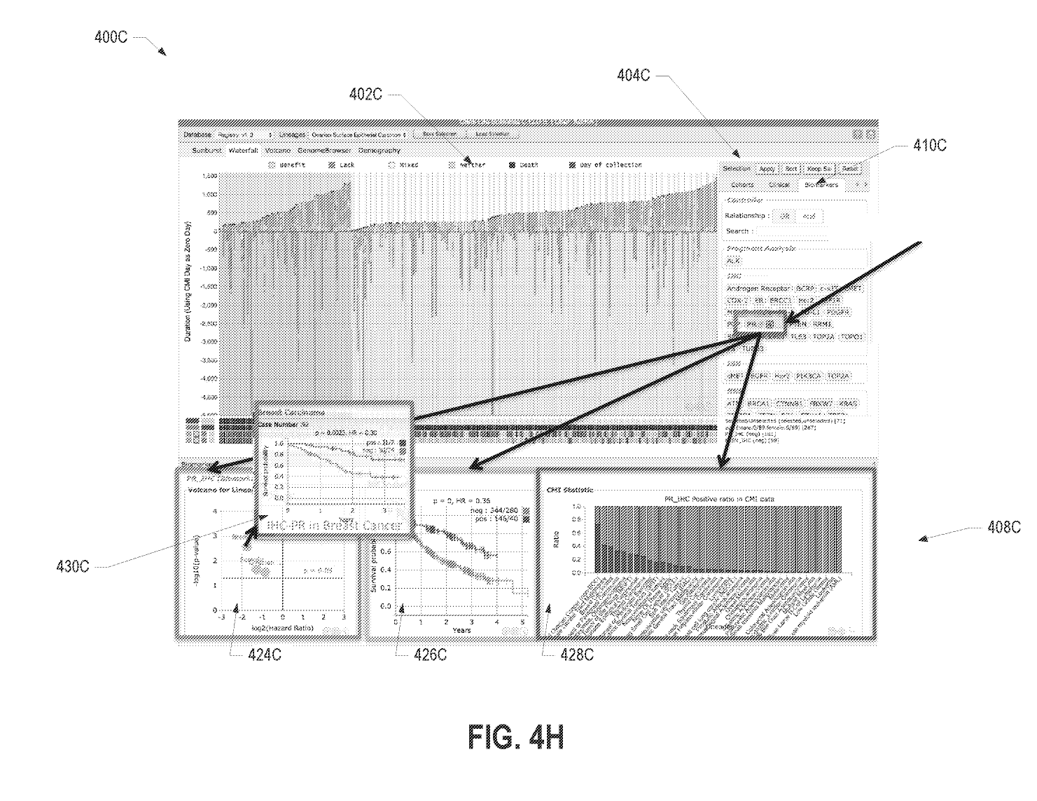

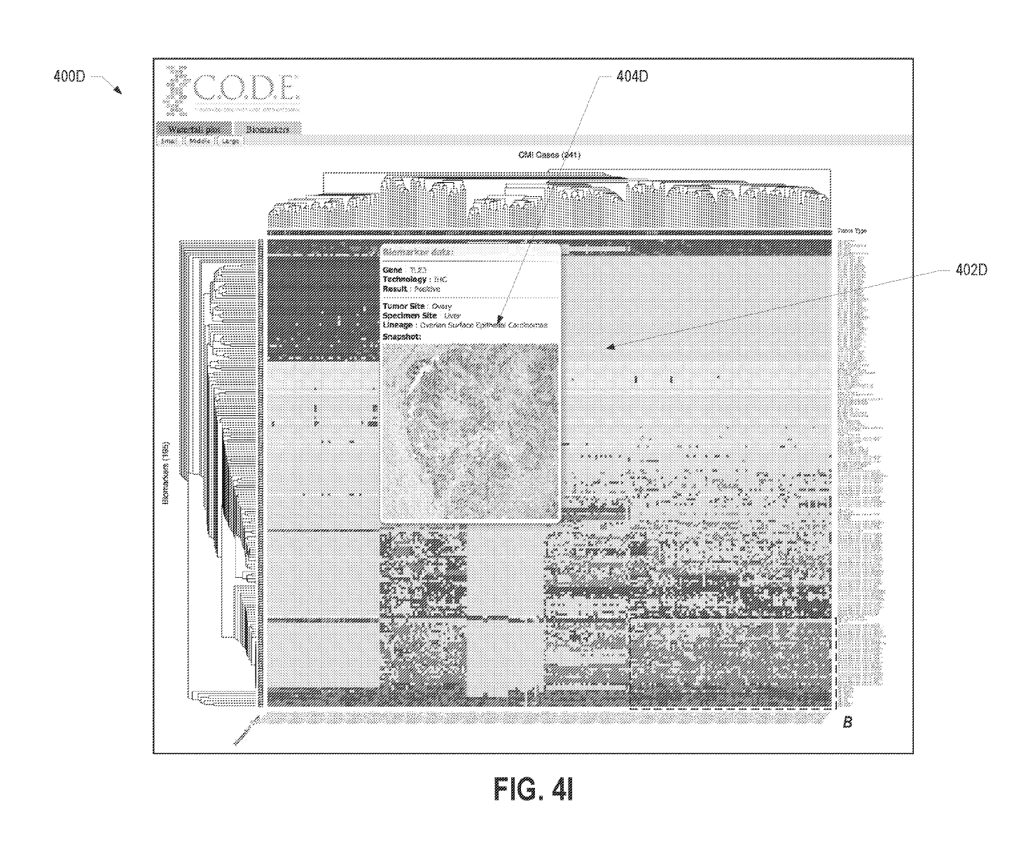

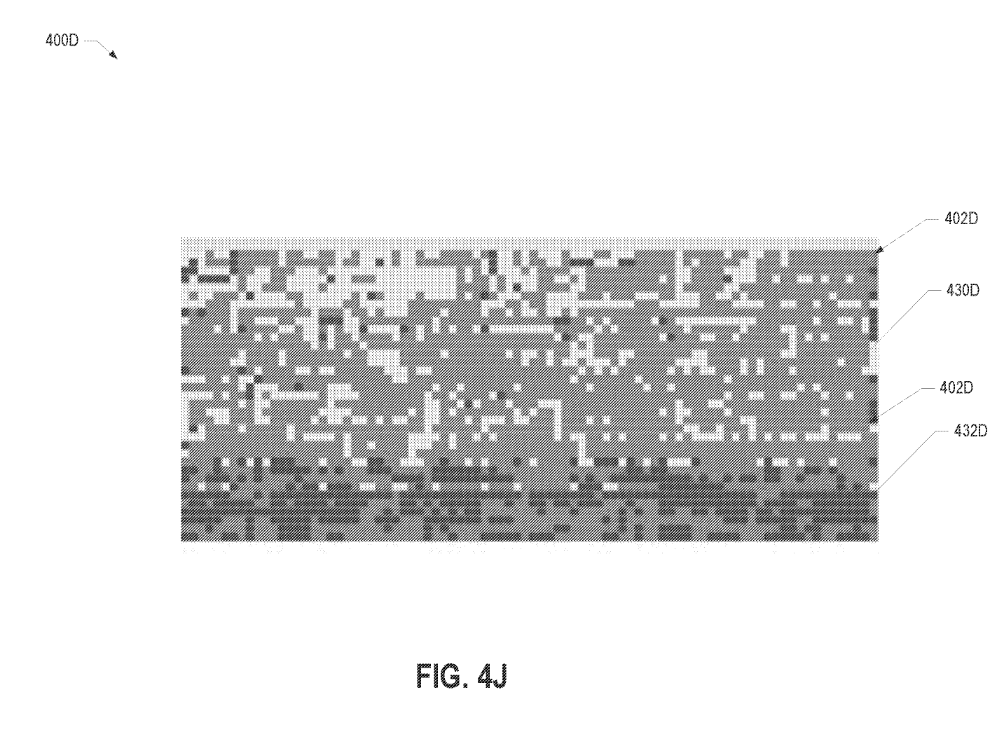

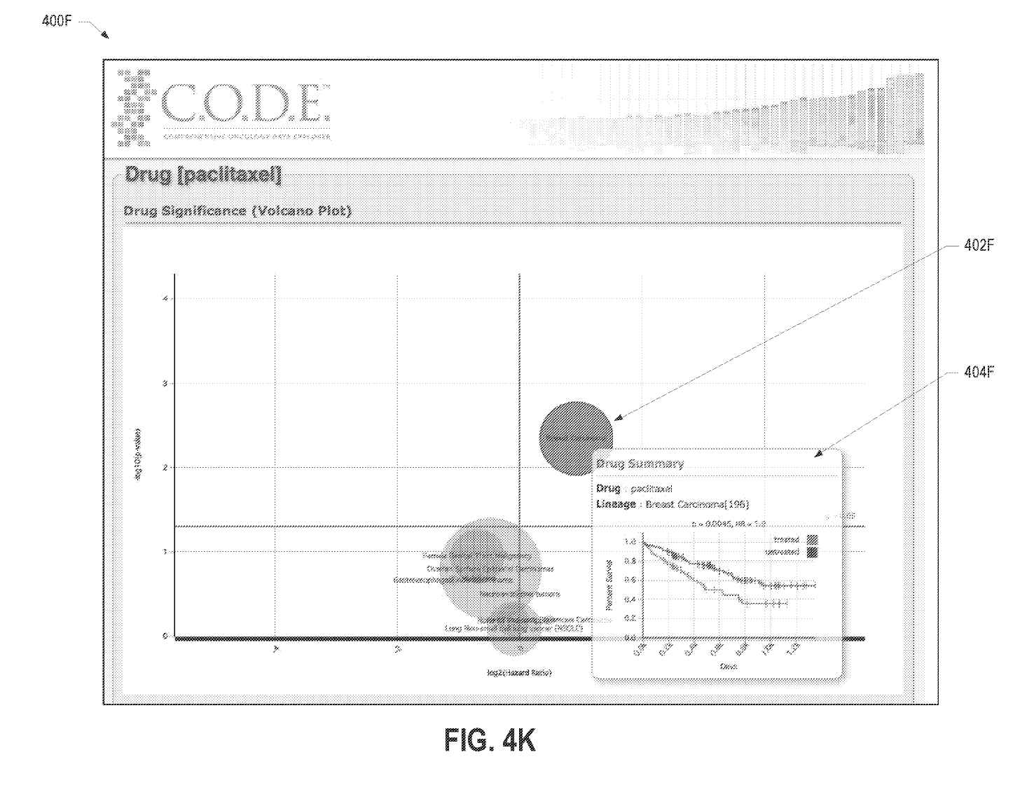

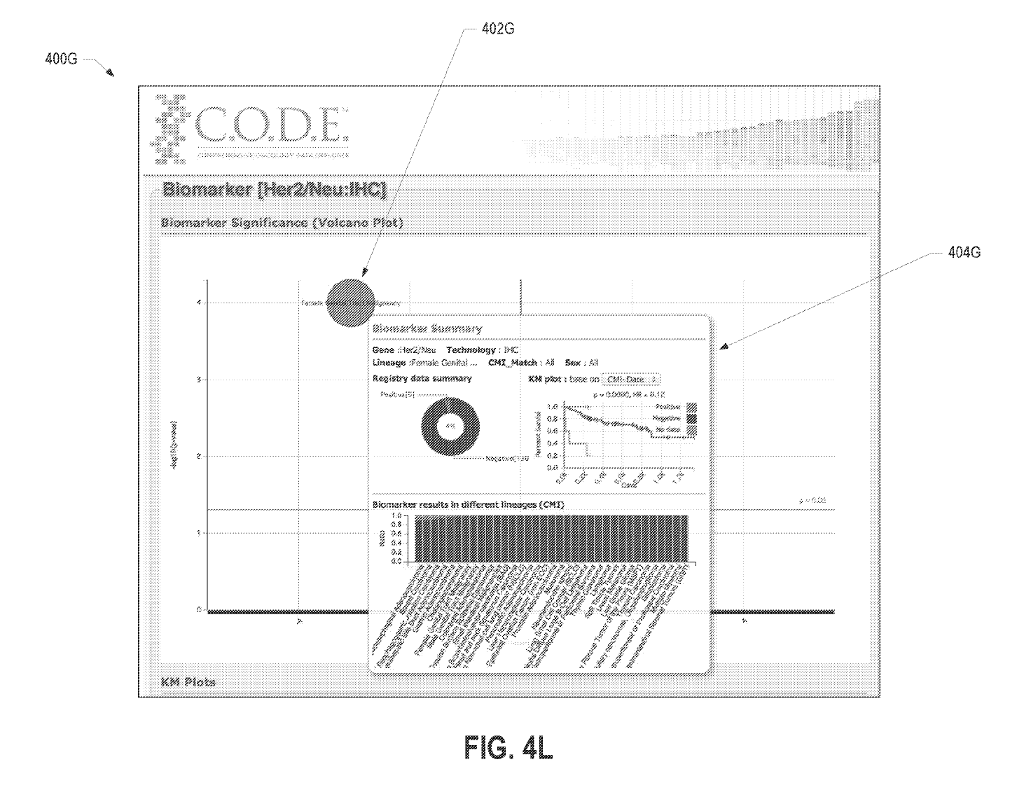

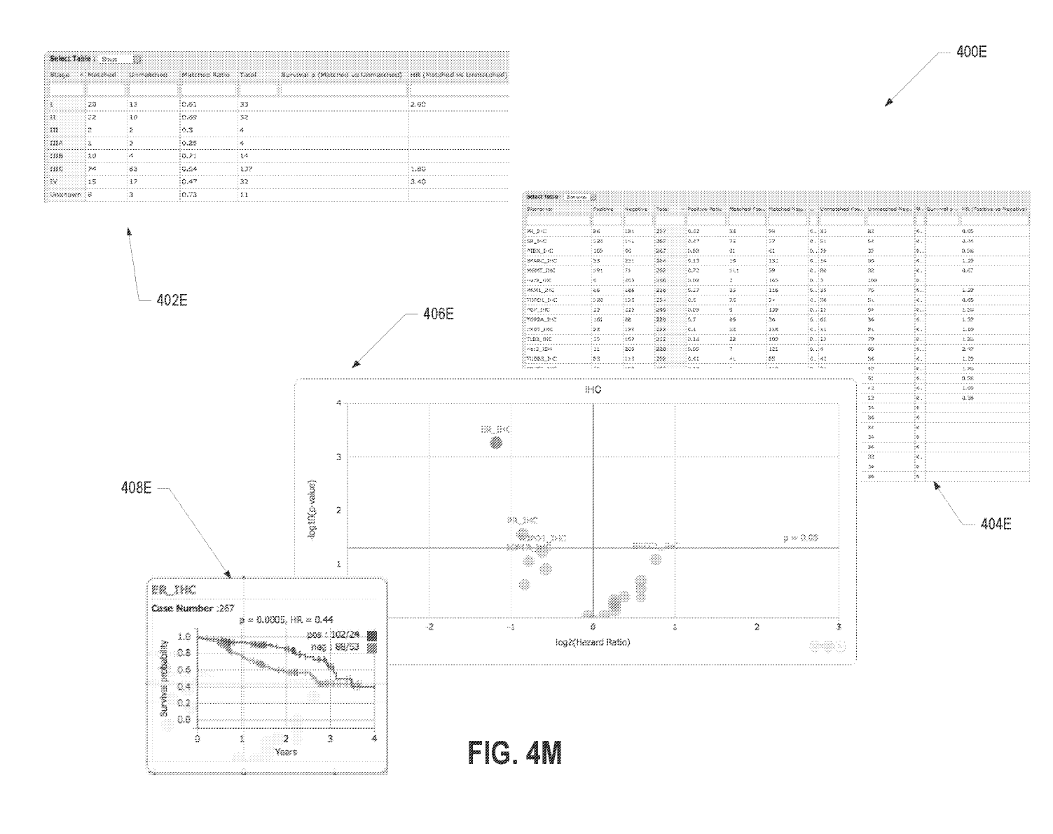

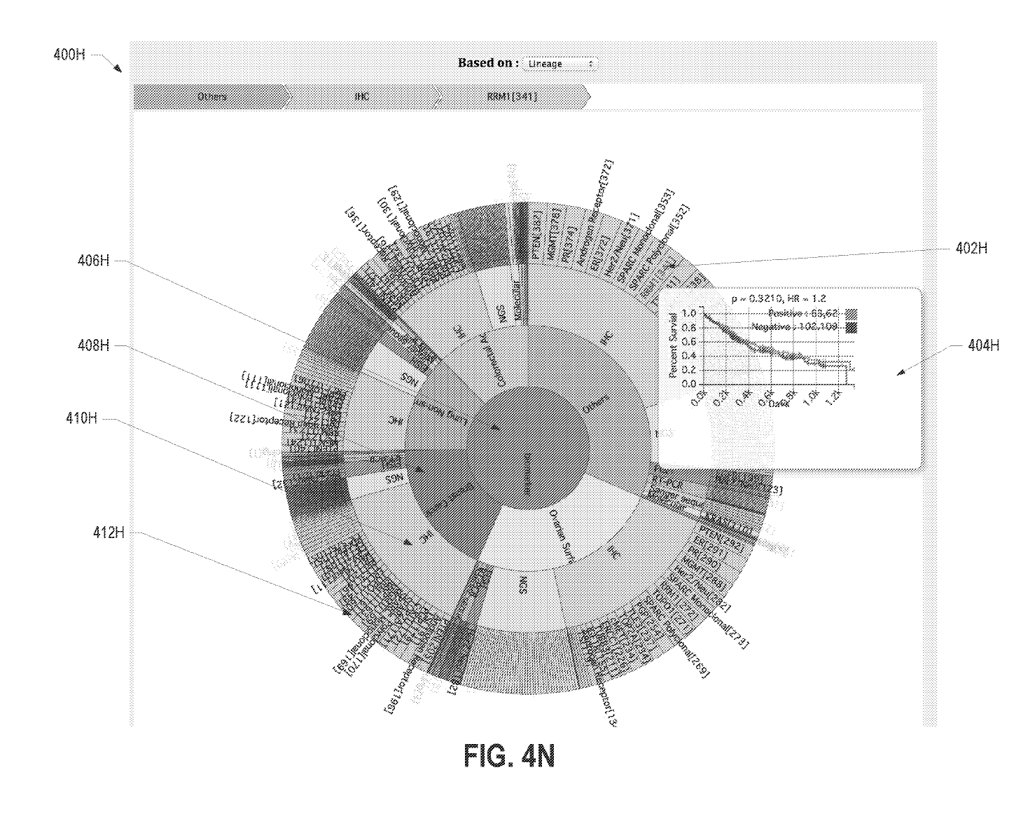

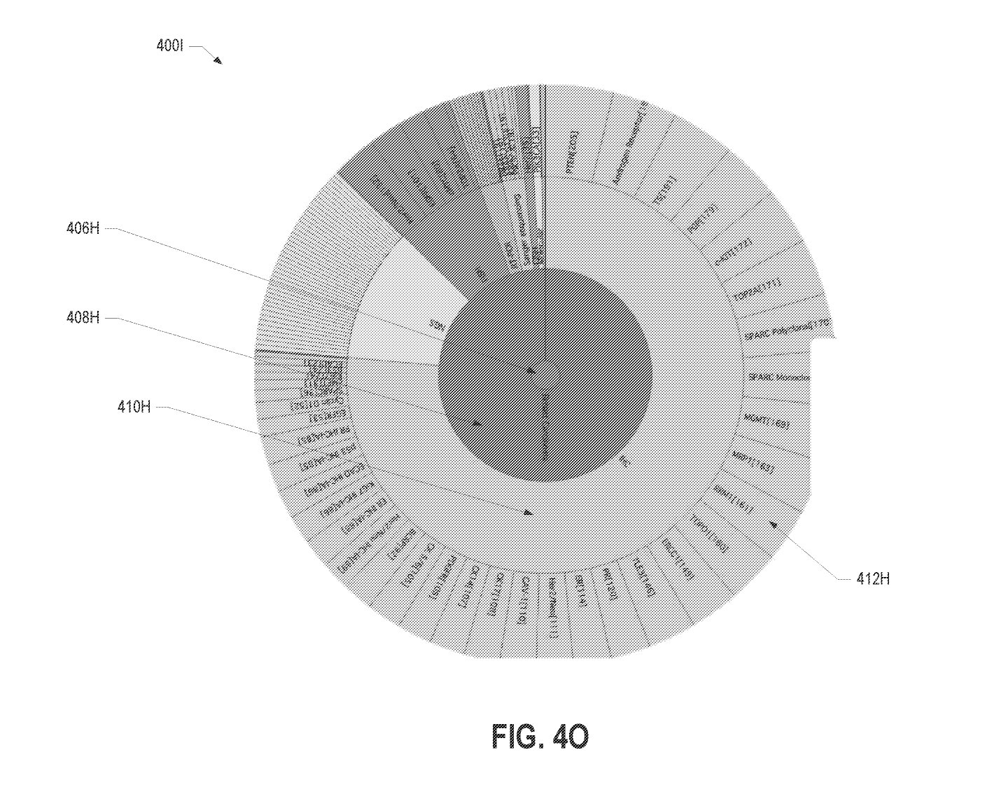

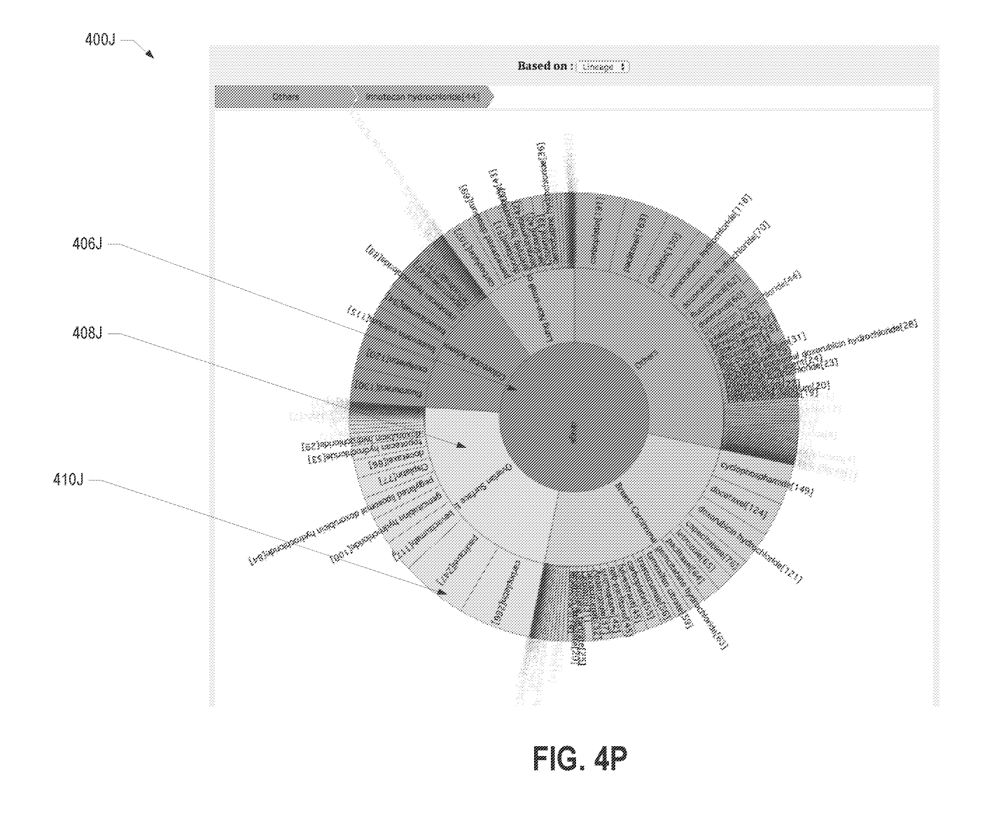

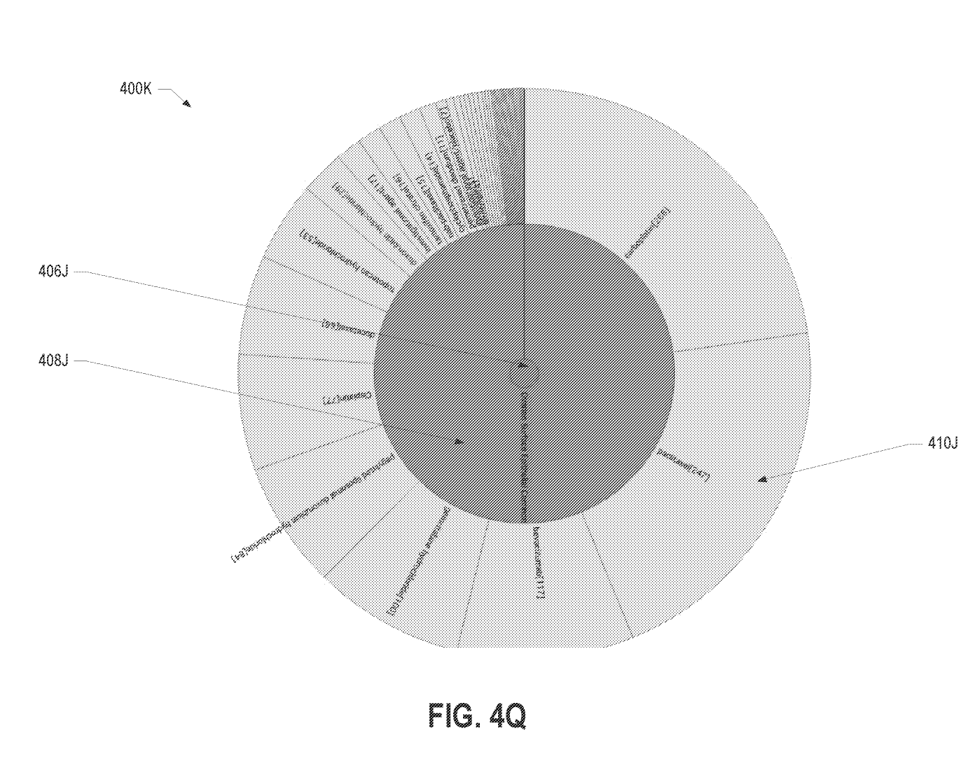

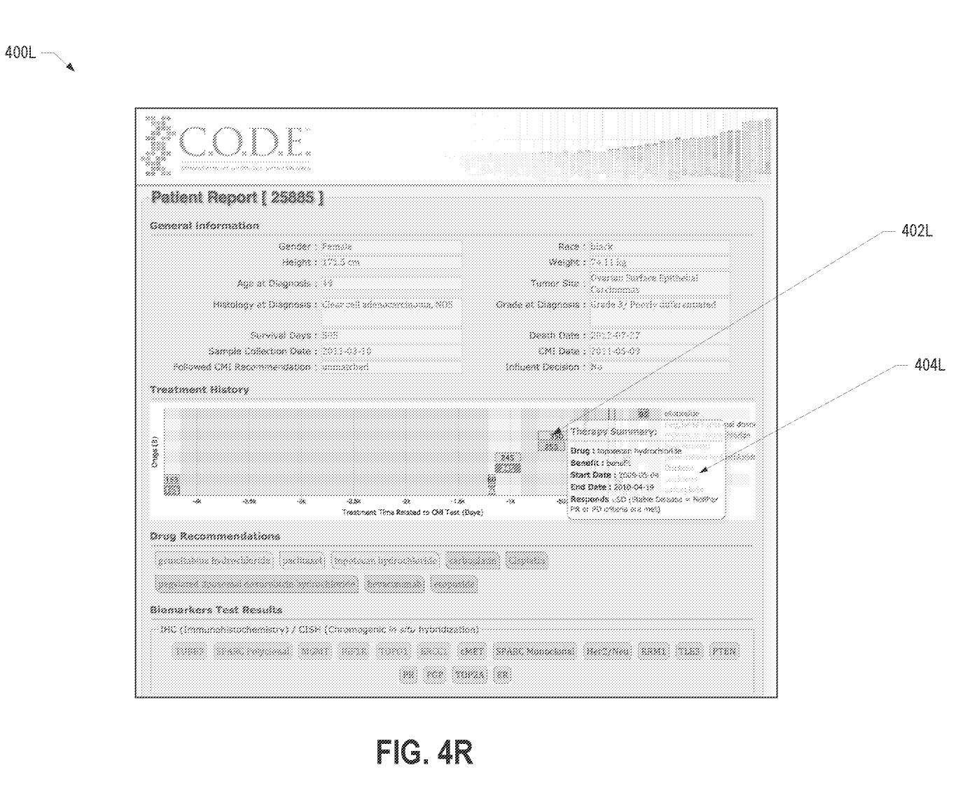

Accordingly, each of FIGS. 4A-4R illustrates an example display of a user interface for an application for analyzing biological data according to one aspect of the present disclosure. The specific data, text, numbers and such textual information provided in each of FIGS. 4A-4R are not necessary as to the inventive aspects provided herein. Such information is merely provided as an example of the kinds, types, and/or quantities of data, text, and/or information that are displayed on a user interface (i.e., the GUI) associated with the application for analyzing the biological data.

### DETAILED DESCRIPTION OF THE DISCLOSURE

The present disclosure now will be described more fully hereinafter with reference to the accompanying drawings, in which some, but not all aspects of the disclosure are shown. Indeed, the disclosure may be embodied in many different forms and should not be construed as limited to the aspects set forth herein. Like numbers refer to like elements throughout.

Molecular profiling systems and methods have been developed to profile various molecular characteristics of patient samples. Such profiling can be used for various purposes, such as providing diagnostic, prognostic and theranostic information. Diagnosis may refer to the detection, identification or characterization (e.g., staging or determining progress) of an illness, condition, disease or disorder by examination of symptoms and other patient characteristics, such as molecular analysis of patient samples. Prognosis may refer to the likely course or outcome of a condition or illness. For example, an advanced disease with limited treatment options can have a poor prognosis. Theranostics includes diagnostic testing that provides the ability to affect therapy or treatment of a diseased state. Theranostics testing provides a theranosis in a similar manner that diagnostics or prognostic testing provides a diagnosis or prognosis, respectively. By way of non-limiting example, theranosis includes detecting a state of a certain biomarker in a patient sample and making a prediction of a likely efficacy of a treatment option based on the detected state of the biomarker. As used herein, theranostics encompasses any desired form of therapy related testing, including predictive medicine, personalized medicine, integrated medicine, pharmacodiagnostics and Dx/Rx partnering. Therapy related tests can be used to predict and assess drug response in individual subjects in order to provide personalized medicine. Predicting a drug response comprises determining whether a subject is a likely responder or a likely non-responder to a candidate therapeutic agent, e.g., before the subject has been exposed or otherwise treated with the treatment. Assessing a drug response can be monitoring a response to a drug, e.g., monitoring the subject's improvement or lack thereof over a time course after initiating the treatment. Theranostic tests are useful to select a subject for treatment who is particularly likely to benefit from the treatment or to provide an early and objective indication of treatment efficacy in an individual subject.

As opposed to traditional medical approaches wherein patients with similar clinical criteria are lumped together for treatment options, molecular profiling analysis may be used to provide or assist in providing more informed and effective personalized treatment options for patients, resulting in improved patient care and enhanced treatment outcomes.

Molecular profiling can be used to determine one or more treatment regimen for a disease, for example a proliferative disorder such as cancer. As an overview, one or more samples from a patient are collected, including without limitation a tumor sample or bodily fluid. The samples are processed and any number of desired molecular tests is run on the one or more sample. For example, molecular testing can be performed to assess panels of biomarkers comprising proteins or nucleic acids. The states of the biomarkers can be compared to biomarker-drug association rules that map relations between states of various biomarkers and therapeutic agents that are more or less likely to benefit the patient. Thus, the states of the biomarkers are used to help guide treatment regimens for the patients. A report can be generated that comprises listings of the drugs that are predicted to be more likely to benefit the patient, less likely to benefit the patient, or of intermediate benefit. The report may list the biomarkers that were tested, the biomarker states determined, and other desired information such as biomarker descriptions and evidence behind the biomarker-drug association rules. Evidence may be derived from various sources such as scientific literature reports, clinical trials, and prior molecular profiling data. Systems can be constructed to carry out such molecular profiling. The systems may comprise various databases, including without limitation databases comprising reference values for the biomarkers tested, biomarker-drug association rules, and evidence supporting each such rule. The systems can comprise computer implemented instructions to compare the test results against the reference values and rules databases, determine drugs of likely benefit, lack of benefit, or intermediate benefit based on the comparisons, and generate the molecular profiling reports. Treating physicians such as oncologists can use such reports to assist in determining personalized treatment regimens for their patients.

Components of an exemplary molecular profiling system are described herein in Example 1. Systems and methods for molecular profiling can be found in US Patent Publications US20100113299, published May 6, 2010; US20140222443, published Aug. 7, 2014; US20150307947, published Oct. 29, 2015; US20160186266, published Jun. 30, 2016; and US20150024952, published Jan. 22, 2015; U.S. Pat. No. 8,700,335, issued Apr. 15, 2014 and U.S. Pat. No. 8,768,629, issued Jul. 1, 2014; and Int'l Patent Publications WO2015116868, published Aug. 6, 2015, and WO2016141169, published Sep. 9, 2016; each of which patent publications is incorporated herein by reference in its entirety. These publications further describe useful biomarkers and biomarker-drug association rules that can be used to perform the molecular profiling. These publications also provide illustrative molecular profiling reports.

A large amount of data can be generated by molecular profiling of individual patients. For example, data may be generated by profiling of at least hundreds, thousands, or tens of thousands patients. Such composite data may be generated for patients having multiple attributes such as cancers of different lineages, histologies, and stages. The patients may differ along clinical parameters including without limitation age and sex. The composite data can comprise the biomarkers and biomarker states determined for the patient samples. As available, data can also be collected for treatment regimens that were actually prescribed to the patients both before and after the time of molecular profiling. Patient response to each treatment can be recorded and tracked over time to create a repository of outcomes data for the patients. The outcomes data can track whether patients were treated with regimens predicted to be of likely benefit according to the molecular profiling (which may be referred to herein as “matched” treatments), treated with regimens predicted to likely not be of benefit according to the molecular profiling (which referred to herein as “unmatched” treatments), and patients whose treatments were of indeterminate benefit or not reported according to the molecular profiling. Systems can be implemented to dynamically add additional patient molecular profile data and outcomes data as such data becomes available.

It will be appreciated that such biomarker and outcome data can provide invaluable knowledge towards the treatment of additional patients. For example, oncologists may treat patients with treatment regimens that were beneficial to other patients having similar molecular profiles. Such data may also be useful for hypothesis generation. For example, molecular profiling data reveals the incidence of states of various biomarkers in various disease settings, and can be mined to generate hypotheses about disease etiology and drug targets. It will be appreciated by one of skill that mining such data with hundreds of parameters (e.g., different biomarkers, states of biomarkers, molecular techniques, drugs, clinical parameters) across patient cohorts with tens or thousands of patients can be very complex. The present invention provides methods and systems that can be used to visualize and analyze complex molecular profiling and outcomes data. Accordingly, the present invention provides improvements in the fields of life sciences and medical practice and research including such aspects as assisting in patient treatment and hypothesis generation. See, e.g., Examples 2 and 3 herein.

As used herein, the terms “data” and “information” and similar terms may be used interchangeably to refer to data capable of being stored, transmitted, received, and/or displayed in accordance with various aspects of the present disclosure. Thus, use of any such terms should not be taken to limit the nature and/or scope of the disclosure.

Additionally, the term “computer-readable medium” as used herein refers to any medium configured to provide and/or assist in providing information such as, for example instructions for execution, to a processor. Computer-readable mediums may take many forms, including, but not limited to a non-transitory computer-readable storage medium (e.g., non-volatile memory, volatile memory, etc.), a transmission medium, and/or the like. Examples of non-transitory computer-readable media include a magnetic computer readable medium (e.g., a floppy disk, a hard disk, magnetic tape, and/or the like), an optical computer readable medium (e.g., a compact disc read only memory (CD-ROM), a digital versatile disk (DVD), a Blu-Ray disc, and/or the like), a random access memory (RAM), a programmable read only memory (PROM), an erasable programmable read only memory (EPROM), a FLASH-EPROM, and/or any other suitable non-transitory medium from which a computer can read. One skilled in the art may appreciate that where aspects are described as using a computer-readable storage medium, other types of computer-readable media may be substituted for or used in addition to the computer-readable storage medium in additional aspects. The term computer-readable storage medium is used herein to refer to any computer-readable medium except transmission media.

In this regard, transmission media may include wired and/or wireless transmission media such as, for example coaxial cables, copper wire, and carrier waves that travel through space without wires and/or cables. Carrier waves may include acoustic waves and electromagnetic waves, which may include radio, optical and infrared waves. Signals include man-made transient variations in amplitude, frequency, phase, polarization, and/or other physical properties transmitted through the transmission medium.

Additionally, as used herein, the term “circuitry” may refer to (1) hardware-only circuit implementations; (2) combinations of circuits and computer program product(s) comprising software and/or firmware instructions stored on one or more computer-readable media that work together to cause an apparatus to perform one or more functions described herein; and/or (3) circuits such as, for example, microprocessor(s) or portion(s) of microprocessor(s) that require software or firmware for operation even if the software or firmware is not physically present. The definition of “circuitry” applies to all uses of the term herein, including in any of the claim(s). As a further example, the term “circuitry” may include, for example, a baseband integrated circuit or applications processor integrated circuit for a mobile device or similar integrated circuit in a server, a cellular network device, and/or other computing and/or network device.

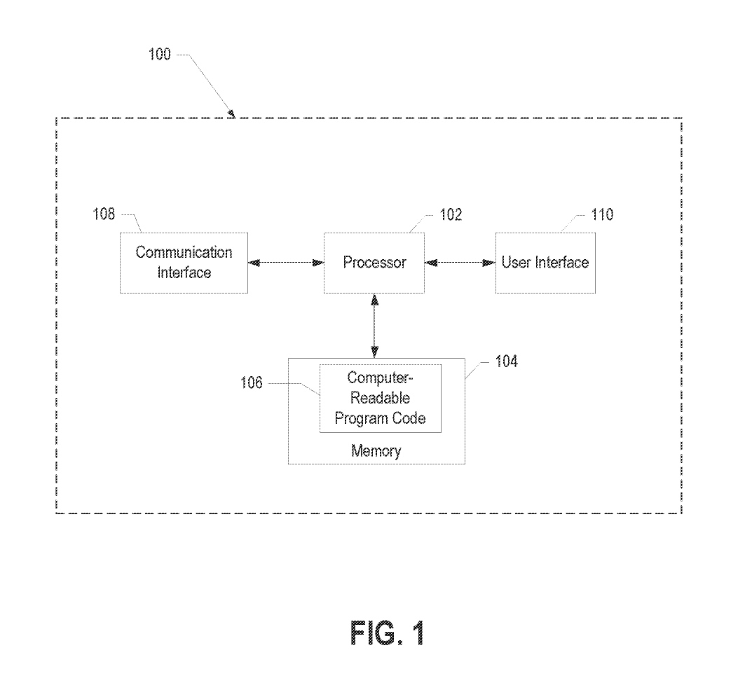

FIG. 1 illustrates a block diagram of an apparatus 100 that may be configured to provide a graphical user interface (GUI) for an application in accordance with various example aspects. The apparatus 100 may be embodied as any computing device or plurality of computing devices that may execute and/or otherwise facilitate usage of an application for which a GUI may be provided for analyzing biological data. According to some example embodiments, the apparatus 100 may be embodied as a server, desktop computer, a laptop computer, a mobile computing device (e.g., a smart phone, a tablet computer, and/or the like), or the like. It will be appreciated that the components, devices, and/or elements illustrated in and described with respect to FIG. 1 below may not be mandatory, and thus, some components, devices and/or elements may be omitted in various embodiments. Additionally, some embodiments may include additional or different components, devices, and/or elements beyond those illustrated in and described with respect to FIG. 1.

In some example embodiments, a processor 102 may be embodied in various forms. For example, the processor 102 may be embodied as various hardware processing means such as a microprocessor, a coprocessor, a controller or various other computing or processing devices including integrated circuits such as, for example, an ASIC (application specific integrated circuit), an FPGA (field programmable gate array), some combination thereof, or the like. Although illustrated as a single processor, it will be appreciated that the processor 102 may comprise a plurality of processors. The plurality of processors may be in operative communication with each other and may be collectively configured to perform one or more functionalities of the apparatus 100. In some embodiments in which the apparatus 100 is embodied as a plurality of computing devices and/or in some embodiments in which at least some functionalities attributed to the apparatus 100 may be performed and/or supported by a remote computing device(s), such as in embodiments in which an application for which a GUI may be provided, is at least partially hosted by a computing device(s) located remotely from an application user (e.g., by a cloud computing infrastructure), a plurality of processors, which may collectively form the processor 102, may be distributed across a plurality of computing devices that may be in operative communication with each other, such as via a network. In some example embodiments, the processor 102 may be configured to execute instructions that may be stored in a memory 104 or that may be otherwise accessible to the processor 102. As such, whether configured by hardware or by a combination of hardware and software, the processor 102 may be capable of performing operations according to various embodiments disclosed herein while configured accordingly.

In some example embodiments, the memory 104 may include one or more memory devices. Memory 104 may include fixed and/or removable memory devices. In embodiments in which the memory 104 includes a plurality of memory devices, the plurality of memory devices may be embodied on a single computing device, or may be distributed across a plurality of computing devices, which may collectively provide functionality of the apparatus 100. For example, in some example embodiments in which an application for which a GUI for analyzing biological data may be provided in accordance with various example embodiments is at least partially remotely hosted (e.g., by a cloud computing infrastructure), the memory 104 may include one or more memory devices that may be disposed remotely from an application user, such as in a server or other cloud computing infrastructure that may host the application. In some embodiments, the memory 104 may provide a non-transitory computer-readable storage medium that may store computer program instructions (e.g., computer-readable program code 106) that may be executed by the processor 102. In this regard, the memory 104 may be configured to store information, data, applications, instructions and/or the like for enabling the apparatus 100 to carry out various functions in accordance with one or more example embodiments. In some embodiments, the memory 104 may be in communication with one or more of the processor 102, communication interface 108, or user interface 110 via a bus(es) for passing information among components of the apparatus 100.

The apparatus 100 may further include a communication interface 108. The communication interface 108 may enable the apparatus 100 to receive a signal that may be sent by another computing device, such as over a network. In this regard, the communication interface 108 may include one or more interface mechanisms for enabling communication with other devices and/or networks. In some example embodiments, the communication interface 108 may include, for example, an antenna (or multiple antennas) and supporting hardware and/or software for enabling communications with a wireless communication network (e.g., a cellular network, Wi-Fi, WLAN, and/or the like) and/or a communication modem or other hardware/software for supporting communication via cable, digital subscriber line (DSL), USB, FireWire, Ethernet or other wireline networking methods. In some example embodiments, the communication interface 108 may be configured to send data to and/or receive data from one or more remote devices and/or networks in support of an application for which a GUI may be provided for analyzing biological data in accordance with one or more example embodiments. Thus, in some example embodiments, data can be communicated to and/or received from another device over a network via communication interface 108 in response to user interaction with a GUI object in accordance with various example embodiments. As an example, in some example embodiments in which a GUI object may be provided for a biological data visualization application, the GUI object may be used to select a parameter for visualization, analysis, and/or the like to apparatus 100 from a remote content source (e.g., a database) via a network, and the selected parameter may be received from the remote content source via the communication interface 108.

In some example embodiments, the apparatus 100 may include the user interface 110. The user interface 110 may be in communication with the processor 102, memory 104, and/or communication interface 108 to receive an indication of a user input and/or to provide an audible, visual, mechanical, or other output to a user. As such, the user interface 110 may include, for example, a keyboard, a mouse, a joystick, a display, a touch screen display, a microphone, a speaker, and/or other input/output mechanisms. In embodiments wherein the user interface 110 comprises a touch screen display, the user interface 110 may additionally be configured to detect and/or receive an indication of a touch gesture or other input to the touch screen display. In some example embodiments, the user interface 110 may comprise a display configured to display a GUI object in accordance with various example embodiments. For example, the user interface 110 of some example embodiments may comprise a display that may be configured to display a plurality of GUI objects that are associated with patient data in accordance with various example embodiments. In some example embodiments in which the user interface 110 comprises a display configured to display a GUI object, the user interface 110 may be configured to detect user input defining an interaction with the GUI object, such as a user input corresponding to a selection of a particular GUI object.

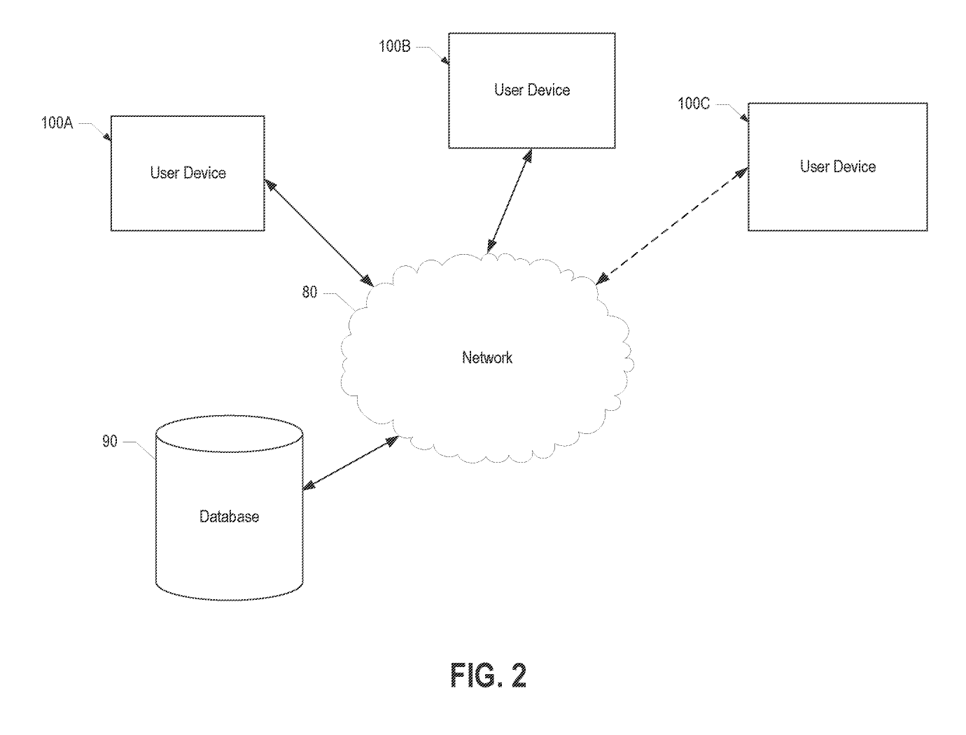

FIG. 2 illustrates a schematic block diagram of a system configured to provide a GUI for an application for analyzing biological data in accordance with various example embodiments of the present disclosure. For example, the user devices 100A, 100B, 100C may include components, devices, and/or elements included within the apparatus 100 as shown in FIG. 1 and described herein. Additionally, some user devices may include additional or different components, devices, and/or elements beyond those illustrated in and described with respect to FIG. 1.

As previously mentioned, the user devices 100A, 100B, 100C may include a communication interface 108 configured to communicate with any of the user devices and/or a database 90 over a network 80. The network 80 may, for example, comprise a wireline network, wireless network (e.g., a cellular network, wireless local area network, wireless wide area network, some combination thereof, or the like), or a combination thereof, and in some example embodiments may comprise the Internet.

The database 90 may be embodied as one or more servers, a cloud computing infrastructure, or the like, which may be configured to provide access to patient data corresponding to patient(s) to one or more user devices 100A, 100B, 100C via the network 80. In some example embodiments, the database 90 may be configured to store information, data, applications, instructions and/or the like for enabling the user devices to carry out various functions in accordance with one or more example embodiments. For example, the database may be configured to store patient data for a plurality of patients. In some embodiments, the patient data is associated with at least a biological sampling event, a biological processing event, a therapeutic regime, a marker status, and/or a patient status. Additionally, the patient data may include reference data associated with at least a biological sampling event, a biological processing event, a therapeutic regime, a marker status, and/or a patient status. In further embodiments, the patient data comprises historical data that tracks the patient status over a period of time. The period of time may comprise a period of time long enough to provide significant information regarding the patient's status. For example, the period of time may comprise a two year period, a three year period, a four year period, a five year period, a six year period, a seven year period, or a longer period time, such as the lifetime of the patient. The period can be based on any such period when the patient has an examination. The patient's status may be updated each time the patient comes in for an exam within that time period. Thus, patient data may comprise outcome data for the patient after one or more therapeutic regimens. Accordingly, the database 90 may be embodied as any suitable computing device and/or infrastructure configured to transmit patient data to any one of the user devices 100A, 100B, 100C via the network 80.

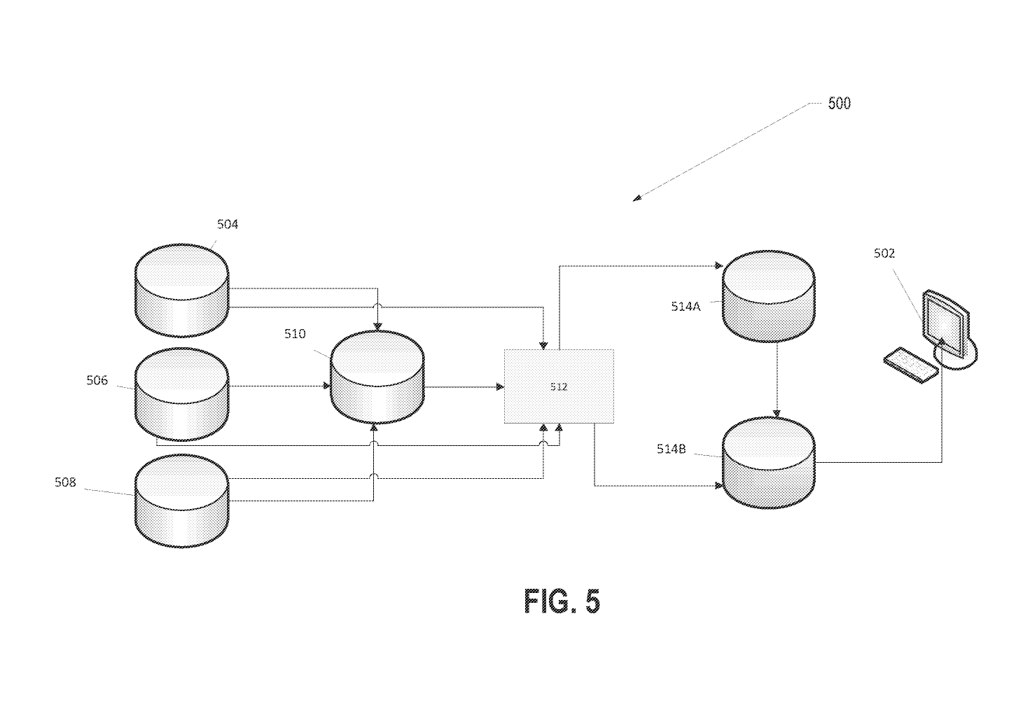

In some example embodiments, the database 90 may be configured to store and/or provide access to an application to one or more of the user devices 100A, 100B, 100C via the network 80. In this regard, in some example embodiments, functionality of the application for analyzing biological data may be divided between the user device and the database. As an example, in some embodiments, the database 90 may be configured to provide the user device 100A, 100B, 100C with patient data corresponding to a biological sampling event, a biological processing event, a therapeutic regime, a marker status, and/or a patient status. Additionally, the database 90 may be configured to provide data corresponding to a relationship between any one of the biological sampling event(s), biological processing event(s), therapeutic regime(s), marker status(es), and/or patient status(es) to any one of the user devices via the network. According to another embodiment, the database 90 may be configured to determine the existence of and/or the nature of the relationship between any of the biological sampling event(s), biological processing event(s), therapeutic regime(s), marker status(es), and/or patient status(es). Likewise, the database 90 may be configured to receive data corresponding to patient data, as described herein, via the network 80. A more detailed description regarding a data base arrangement including database 90 is provided in reference to FIG. 5.

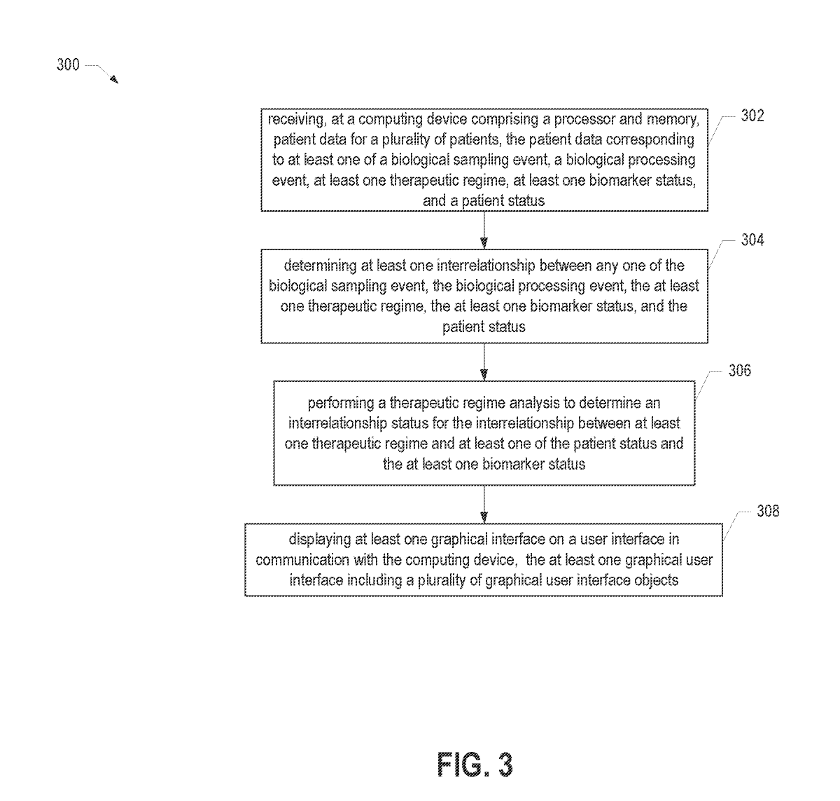

FIG. 3 illustrates a flowchart according to an example method of analyzing biological data according to various example aspects. In particular, the example method 300 of analyzing biological data may be provided via an application having a GUI object according to example embodiments described herein. The method 300 may begin with receiving, at a computing device comprising a processor and memory, patient data, 302, for a plurality of patients. The patient data may correspond to at least one of a biological sampling event, a biological processing event, a therapeutic regime, a marker status (e.g., a biomarker status), and/or a patient status. In some embodiments, the patient data may include reference data corresponding to biological sampling event(s), biological processing event(s), therapeutic regime(s), marker status(es), and/or patient status(es) for a plurality of patients. According to another embodiment, the patient data may include data corresponding to at least one of a biological sampling event, a biological processing event, a therapeutic regime, a marker status, and/or a patient status for a target patient associated with a biological sample to be analyzed.

The method 300 may further include determining at least one interrelationship between any one of the biological sampling event, the biological processing event, the therapeutic regime, the marker status, and the patient status, 304. For example, an apparatus and/or a database may include components, devices, and/or elements to determine the existence of and/or nature of a relationship between a marker status and a patient status. In this regard, a marker status (e.g., biomarker status) may indicate the presence of a particular marker within a biological sample (e.g., tissue, fluid, etc.). Additionally, data corresponding to a patient status may include information associated with patient attributes (e.g., age, sex, race, etc.), the patient's condition and/or disease (i.e., the particular condition and/or disease afflicting the patient), the status of the patient's condition and/or disease (i.e., remission, recurrence, etc.), an outcome of the condition and/or disease (i.e., death, partial remission, complete remission, recurrence, or cure), and/or the like. As such, an apparatus and/or a database may be configured to determine a relationship between a marker status and a patient status. For example, the apparatus and/or database, as described herein, may be configured to determine a relationship exists between the status of a patient's condition and/or disease and a biological sample that has been processed so as to indicate the presence of a particular biomarker.

The biological sample may include any relevant biological sample that can be used for molecular profiling (e.g., sections of tissues such as biopsy or tissue removed during surgical or other procedures, bodily fluids, autopsy samples, and frozen sections taken for histological purposes). Such samples include blood and blood fractions or products (e.g., serum, buffy coat, plasma, platelets, red blood cells, and the like), sputum, malignant effusion, cheek cells tissue, cultured cells (e.g., primary cultures, explants, and transformed cells), stool, urine, other biological or bodily fluids (e.g., prostatic fluid, gastric fluid, intestinal fluid, renal fluid, lung fluid, cerebrospinal fluid, and the like), etc. The sample can comprise biological material that is a fresh frozen & formalin fixed paraffin embedded (FFPE) block, formalin-fixed paraffin embedded, or is within an RNA preservative+formalin fixative. More than one sample of more than one type can be assessed for an individual patient. In an embodiment, the biological sample comprises a tumor sample. The tumor sample may be a fixed tumor sample.

The marker/biomarker can be any useful biological molecule or entity, including without limitation a protein (including a polypeptide or peptide), nucleic acid, lipid, carbohydrate, or a combination of any combination thereof. Nucleic acids include without limitation deoxyribonucleic acid (DNA) and ribonucleic acids (RNA), such as messenger RNA (mRNA), transfer RNA (tRNA), small RNAs, non-coding RNAs, and microRNAs. Any useful characteristic can be determined for a marker/biomarker, including without limitation a concentration, expression level, copy number, amino acid or nucleic acid sequence. Sequences can be assessed for various characteristics, including without limitation at least one of a mutation, a polymorphism, a deletion, an insertion, a substitution, a translocation, a fusion, a break, a duplication, an amplification, a repeat, a copy number variant (CNV), a DNA methylation variation, a transcript expression level, a transcript variant, and a splice variant.

A marker/biomarker status can be determined by any appropriate laboratory technique for assessing a molecule in a biological sample. The technique may comprise gene expression analysis, nucleic acid sequence analysis, nucleic acid methylation analysis and/or proteomic analysis. Techniques for assessing such markers include but are not limited to, nucleic acid sequencing, such as a DNA sequencing or RNA sequencing; protein immunoassays such as Western blots, ELISA or immunohistochemistry (IHC); nucleic acid analysis such in situ hybridization (ISH), including fluorescent in situ hybridization (FISH) and/or chromogenic in situ hybridization (CISH); nucleic acid amplification (e.g., polymerase chain reaction (PCR), and quantitative varieties thereof including qPCR or RT-PCR); various types of microarray (mRNA expression arrays, PCR-based low density arrays, protein arrays, etc); various types of nucleic acid sequencing (Sanger, pyrosequencing, etc); comparative genomic hybridization (CGH); high throughput sequencing (HTS) or Next Generation sequencing (NGS) of nucleic acids; Northern blot for RNA; Southern blot for DNA; flow cytometry; nucleic acid methylation analysis; nucleic acid fragment analysis; gel electrophoresis; and any other appropriate technique to assay the presence or quantity of a biological molecule of interest. In various embodiments of the invention, any one or more of these techniques are used concurrently or subsequent to each other for assessing markers of interest.

Additional description of useful samples and biomarker analysis techniques can be found in US Patent Publications US20100113299, published May 6, 2010; US20140222443, published Aug. 7, 2014; US20150307947, published Oct. 29, 2015; US20160186266, published Jun. 30, 2016; and US20150024952, published Jan. 22, 2015; U.S. Pat. No. 8,700,335, issued Apr. 15, 2014 and U.S. Pat. No. 8,768,629, issued Jul. 1, 2014; and Int'l Patent Publications WO2015116868, published Aug. 6, 2015, and WO2016141169, published Sep. 9, 2016; each of which patent publications is incorporated herein by reference in its entirety.

The technique used to assess a marker can be chosen by the evidence that links a characteristic of that marker to a diagnosis, prognosis and/or theranosis. In one non-limiting example, it is known that the protein level and DNA copy number of the HER2/ERBB2 gene and protein are related to the efficacy of anti-HER2 treatments such as trastuzumab, ado-trastuzumab emtansine, pertuzumab or lapatinib. Thus, one may choose to assess HER2 using IHC at the protein level or ISH to assess HER2 gene copy number.

Any number of markers can be assessed according to the invention. The markers may be chosen to relate to a diagnosis, prognosis and/or theranosis of a condition and/or disease such as a cancer. In some embodiments, the markers comprise at least one of 1p19q, ABL1, AKT1, ALK, APC, AR, ATM, BRAF, BRCA1, BRCA2, cKIT, cMET, CSF1R, CTNNB1, EGFR, EGFRvIII, ER, ERBB2 (HER2), ERCC1, FGFR1, FGFR2, FLT3, GNA11, GNAQ, GNAS, H3K36me3, HER2, HRAS, IDH1, IDH2, JAK2, KDR (VEGFR2), KRAS, MDM2, MGMT, MLH1, MPL, NOTCH1, NRAS, PBRM1, PD1, PDL1, PDGFRA, Pgp, PIK3CA, PR, PTEN, RET, RRM1, SMO, SPARC, TLE3, TOP2A, TOPO1, TP53, TS, TUBB3, VHL, MLH1, MSH2, MSH6, PMS2, microsatellite instability (MSI) and ROS1. The markers may also include at least one of CAIX, hENT1, IDO, LAG3, RET, and NTRK1 (NTRK, TRK). Any of the markers can be assessed using any appropriate laboratory technique disclosed above or known in the art.

The markers may include at least one, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or 46, marker selected from ABL1, AKT1, ALK, APC, ATM, BRAF, BRCA1, BRCA2, CDH1, cKIT, cMET, CSF1R, CTNNB1, EGFR, ERBB2 (Her2), ERBB4, FBXW7, FGFR1, FGFR2, FLT3, GNA11, GNAQ, GNAS, HNF1A, HRAS, IDH1, JAK2, JAK3, KDR (VGFR2), KRAS, MPL, NOTCH1, NPM1, NRAS, PDGFRA, PIK3CA, PTEN, PTPN11, RB1, RET, SMAD4, SMARCB1, SMO, STK11, TP53, and VHL. These markers may be assessed at the DNA sequence level, e.g., including without limitation at least one of a mutation, a polymorphism, a deletion, an insertion, a substitution, a translocation, a fusion, a break, a duplication, an amplification, a repeat, a copy number variant (CNV), a DNA methylation variation, a transcript expression level, a transcript variant, and a splice variant.

In other embodiment, nucleic acid sequence analysis is used to assess at least one gene, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 250, 300, 350, 400, 450, 500, or all genes, selected from the group consisting of ABI1, ABL2, ACSL3, ACSL6, AFF1, AFF3, AFF4, AKAP9, AKT2, AKT3, ALDH2, AMER1, AR, ARFRP1, ARHGAP26, ARHGEF12, ARID1A, ARID2, ARNT, ASPSCR1, ASXL1, ATF1, ATIC, ATP1A1, ATP2B3, ATR, ATRX, AURKA, AURKB, AXIN1, AXL, BARD1, BCL10, BCL11A, BCL11B, BCL2, BCL2L11, BCL2L2, BCL3, BCL6, BCL7A, BCL9, BCOR, BCORL1, BCR, BIRC3, BLM, BMPR1A, BRD3, BRD4, BRIP1, BTG1, BTK, BUB1B, C11orf30, C15orf21, C15orf55, C15orf65, C16orf75, C2orf44, CACNA1D, CALR, CAMTA1, CANT1, CARD11, CARS, CASC5, CASP8, CBFA2T3, CBFB, CBL, CBLB, CBLC, CCDC6, CCNB1IP1, CCND1, CCND2, CCND3, CCNE1, CD274, CD74, CD79A, CD79B, CDC73, CDH11, CDK12, CDK4, CDK6, CDK8, CDKN1B, CDKN2A, CDKN2B, CDKN2C, CDX2, CEBPA, CHCHD7, CHIC2, CHN1, CIC, CIITA, CLP1, CLTC, CLTCL1, CNBP, CNOT3, CNTRL, COL1A1, COPB1, COX6C, CREB1, CREB3L1, CREB3L2, CREBBP, CRKL, CRLF2, CRTC1, CRTC3, CSF3R, CTCF, CTLA4, CTNNA1, CXCR7, CYLD, CYP2D6, DAXX, DDB2, DDIT3, DDX10, DDX5, DDX6, DEK, DICER1, DNM2, DNMT3A, DOT1L, DUX4, EBF1, ECT2L, EIF4A2, ELF4, ELK4, ELL, ELN, EML4, EP300, EPHA3, EPHA5, EPHB1, EPS15, ERC1, ERCC1, ERCC2, ERCC3, ERCC4, ERCC5, ERG, ESR1, ETV1, ETV4, ETV5, ETV6, EWSR1, EXT1, EXT2, EZH2, EZR, FAM123B, FAM22A, FAM22B, FAM46C, FANCA, FANCC, FANCD2, FANCE, FANCF, FANCG, FANCL, FAS, FBXO11, FCGR2B, FCRL4, FEV, FGF10, FGF14, FGF19, FGF23, FGF3, FGF4, FGF6, FGFR1OP, FGFR3, FGFR4, FH, FHIT, FIP1L1, FLCN, FLI1, FLT1, FLT4, FNBP1, FOXA1, FOXL2, FOXO1, FOXO3, FOXO4, FOXP1, FSTL3, FUBP1, FUS, GAS7, GATA1, GATA2, GATA3, GID4, GMPS, GNA13, GOLGA5, GOPC, GPC3, GPHN, GPR124, GRIN2A, GSK3B, H3F3A, H3F3B, HERPUD1, HEY1, HGF, HIP1, HIST1H3B, HIST1H4I, HLF, HMGA1, HMGA2, HNRNPA2B1, HOOK3, HOXA11, HOXA13, HOXA9, HOXC11, HOXC13, HOXD11, HOXD13, HSP90AA1, HSP90AB1, IGF1R, IKBKE, IKZF1, IL2, IL21R, IL6ST, IL7R, INHBA, IRF4, IRS2, ITK, JAK1, JAZF1, JUN, KAT6A, KCNJ5, KDM5A, KDM5C, KDM6A, KDSR, KEAP1, KIAA1549, KIF5B, KLF4, KLHL6, KLK2, KTN1, LASP1, LCK, LCP1, LGR5, LHFP, LIFR, LMO1, LMO2, LPP, LRIG3, LRP1B, LYL1, MAF, MAFB, MALT1, MAML2, MAP2K1 (MEK1), MAP2K2 (MEK2), MAP2K4, MAP3K1, MAX, MCL1, MDM2, MDM4, MDS2, MECOM, MED12, MEF2B, MEN1, MITF, MKL1, MLF1, MLL, MLL2, MLL3, MLLT1, MLLT10, MLLT11, MLLT3, MLLT4, MLLT6, MN1, MNX1, MRE11A, MSH2, MSH6, MSI2, MSN, MTCP1, MTOR, MUC1, MUTYH, MYB, MYC, MYCL1, MYCN, MYD88, MYH11, MYH9, MYST4, NACA, NBN, NCKIPSD, NCOA1, NCOA2, NCOA4, NDRG1, NF2, NFE2L2, NFIB, NFKB2, NFKBIA, NIN, NKX2-1, NONO, NOTCH2, NR4A3, NSD1, NT5C2, NTRK2, NTRK3, NUMA1, NUP214, NUP93, NUP98, OLIG2, OMD, P2RY8, PAFAH1B2, PAK3, PALB2, PATZ1, PAX3, PAX5, PAX7, PAX8, PBRM1, PBX1, PCM1, PCSK7, PDCD1, PDCD1LG2, PDE4DIP, PDGFB, PDGFRB, PDK1, PER1, PHF6, PHOX2B, PICALM, PIK3CG, PIK3R1, PIK3R2, PIM1, PLAG1, PML, PMS1, PMS2, POLE, POT1, POU2AF1, POU5F1, PPARG, PPP2R1A, PRCC, PRDM1, PRDM16, PRF1, PRKAR1A, PRKDC, PRRX1, PSIP1, PTCH1, PTPRC, RABEP1, RAC1, RAD21, RAD50, RAD51, RAD51L1, RALGDS, RANBP17, RAP1GDS1, RARA, RBM15, RECQL4, REL, RHOH, RICTOR, RNF213, RNF43, RPL10, RPL22, RPL5, RPN1, RPTOR, RUNDC2A, RUNX1, RUNx1T1, SBDS, SDC4, SDHAF2, SDHB, SDHC, SDHD, SEPT5, SEPT6, SEPT9, SET, SETBP1, SETD2, SF3B1, SFPQ, SFRS3, SH2B3, SH3GL1, SLC34A2, SLC45A3, SMAD2, SMARCA4, SMARCE1, SOCS1, SOX10, SOX2, SPECC1, SPEN, SPOP, SRC, SRGAP3, SRSF2, SS18, SS18L1, SSX1, SSX2, SSX4, STAG2, STAT3, STAT4, STAT5B, STIL, SUFU, SUZ12, SYK, TAF15, TAL1, TAL2, TBL1XR1, TCEA1, TCF12, TCF3, TCF7L2, TCL1A, TERT, TET1, TET2, TFE3, TFEB, TFG, TFPT, TFRC, TGFBR2, THRAP3, TLX1, TLX3, TMPRSS2, TNFAIP3, TNFRSF14, TNFRSF17, TOP1, TPM3, TPM4, TPR, TRAF7, TRIM26, TRIM27, TRIM33, TRIP11, TRRAP, TSC1, TSC2, TSHR, TTL, U2AF1, UBR5, USP6, VEGFA, VEGFB, VTI1A, WAS, WHSC1, WHSC1L1, WIF1, WISP3, WRN, WWTR1, XPA, XPC, XPO1, YWHAE, ZBTB16, ZMYM2, ZNF217, ZNF331, ZNF384, ZNF521, ZNF703, ZRSR2.

In another embodiment, nucleic acid sequence analysis is used to assess at least one gene, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 250, 300, 350, 400, 450, 500, or all genes, selected from the group consisting of ABI1, ABL1, ABL2, ACKR3, ACSL3, ACSL6, AFF1, AFF3, AFF4, AKAP9, AKT1, AKT2, AKT3, ALDH2, ALK, AMER1 (FAM123B), APC, AR, ARAF, ARFRP1, ARHGAP26, ARHGEF12, ARID1A, ARID2, ARNT, ASPSCR1, ASXL1, ATF1, ATIC, ATM, ATP1A1, ATP2B3, ATR, ATRX, AURKA, AURKB, AXIN1, AXL, BAP1, BARD1, BCL10, BCL11A, BCL11B, BCL2, BCL2L11, BCL2L2, BCL3, BCL6, BCL7A, BCL9, BCOR, BCORL1, BCR, BIRC3, BLM, BMPR1A, BRAF, BRCA1, BRCA2, BRD3, BRD4, BRIP1, BTG1, BTK, BUB1B, C11orf30 (EMSY), C15orf65, C2orf44, CACNA1D, CALR, CAMTA1, CANT1, CARD11, CARS, CASC5, CASP8, CBFA2T3, CBFB, CBL, CBLB, CBLC, CCDC6, CCNB1IP1, CCND1, CCND2, CCND3, CCNE1, CD274 (PDL1), CD74, CD79A, CD79B, CDC73, CDH1, CDH11, CDK12, CDK4, CDK6, CDK8, CDKN1B, CDKN2A, CDKN2B, CDKN2C, CDX2, CEBPA, CHCHD7, CHEK1, CHEK2, CHIC2, CHN1, CIC, CIITA, CLP1, CLTC, CLTCL1, CNBP, CNOT3, CNTRL, COL1A1, COPB1, COX6C, CREB1, CREB3L1, CREB3L2, CREBBP, CRKL, CRLF2, CRTC1, CRTC3, CSF1R, CSF3R, CTCF, CTLA4, CTNNA1, CTNNB1, CYLD, CYP2D6, DAXX, DDB2, DDIT3, DDR2, DDX10, DDX5, DDX6, DEK, DICER1, DNM2, DNMT3A, DOT1L, EBF1, ECT2L, EGFR, EIF4A2, ELF4, ELK4, ELL, ELN, EML4, EP300, EPHA3, EPHA5, EPHB1, EPS15, ERBB2 (HER2), ERBB3 (HER3), ERBB4 (HER4), ERC1, ERCC1, ERCC2, ERCC3, ERCC4, ERCC5, ERG, ESR1, ETV1, ETV4, ETV5, ETV6, EWSR1, EXT1, EXT2, EZH2, EZR, FAM46C, FANCA, FANCC, FANCD2, FANCE, FANCF, FANCG, FANCL, FAS, FBXO11, FBXW7, FCRL4, FEV, FGF10, FGF14, FGF19, FGF23, FGF3, FGF4, FGF6, FGFR1, FGFR1OP, FGFR2, FGFR3, FGFR4, FH, FHIT, FIP1L1, FLCN, FLI1, FLT1, FLT3, FLT4, FNBP1, FOXA1, FOXL2, FOXO1, FOXO3, FOXO4, FOXP1, FSTL3, FUBP1, FUS, GAS7, GATA1, GATA2, GATA3, GID4 (C17orf39), GMPS, GNA11, GNA13, GNAQ, GNAS, GOLGA5, GOPC, GPC3, GPHN, GPR124, GRIN2A, GSK3B, H3F3A, H3F3B, HERPUD1, HEY1, HGF, HIP1, HIST1H3B, HIST1H4I, HLF, HMGA1, HMGA2, HMGN2P46, HNF1A, HNRNPA2B1, HOOK3, HOXA11, HOXA13, HOXA9, HOXC11, HOXC13, HOXD11, HOXD13, HRAS, HSP90AA1, HSP90AB1, IDH1, IDH2, IGF1R, IKBKE, IKZF1, IL2, IL21R, IL6ST, IL7R, INHBA, IRF4, IRS2, ITK, JAK1, JAK2, JAK3, JAZF1, JUN, KAT6A (MYST3), KAT6B, KCNJ5, KDM5A, KDM5C, KDM6A, KDR, KDSR, KEAP1, KIAA1549, KIF5B, KIT, KLF4, KLHL6, KLK2, KMT2A (MLL), KMT2C (MLL3), KMT2D (MLL2), KRAS, KTN1, LASP1, LCK, LCP1, LGR5, LHFP, LIFR, LMO1, LMO2, LPP, LRIG3, LRP1B, LYL1, MAF, MAFB, MALT1, MAML2, MAP2K1, MAP2K2, MAP2K4, MAP3K1, MAX, MCL1, MDM2, MDM4, MDS2, MECOM, MED12, MEF2B, MEN1, MET, MITF, MKL1, MLF1, MLH1, MLLT1, MLLT10, MLLT11, MLLT3, MLLT4, MLLT6, MN1, MNX1, MPL, MRE11A, MSH2, MSH6, MSI2, MSN, MTCP1, MTOR, MUC1, MUTYH, MYB, MYC, MYCL (MYCL1), MYCN, MYD88, MYH11, MYH9, NACA, NBN, NCKIPSD, NCOA1, NCOA2, NCOA4, NDRG1, NF1, NF2, NFE2L2, NFIB, NFKB2, NFKBIA, NIN, NKX2-1, NONO, NOTCH1, NOTCH2, NPM1, NR4A3, NRAS, NSD1, NT5C2, NTRK1, NTRK2, NTRK3, NUMA1, NUP214, NUP93, NUP98, NUTM1, NUTM2B, OLIG2, OMD, P2RY8, PAFAH1B2, PAK3, PALB2, PATZ1, PAX3, PAX5, PAX7, PAX8, PBRM1, PBX1, PCM1, PCSK7, PDCD1 (PD1), PDCD1LG2 (PDL2), PDE4DIP, PDGFB, PDGFRA, PDGFRB, PDK1, PER1, PHF6, PHOX2B, PICALM, PIK3CA, PIK3CG, PIK3R1, PIK3R2, PIM1, PLAG1, PML, PMS1, PMS2, POLE, POT1, POU2AF1, POU5F1, PPARG, PPP2R1A, PRCC, PRDM1, PRDM16, PRF1, PRKAR1A, PRKDC, PRRX1, PSIP1, PTCH1, PTEN, PTPN11, PTPRC, RABEP1, RAC1, RAD21, RAD50, RAD51, RAD51B, RAF1, RALGDS, RANBP17, RAP1GDS1, RARA, RB1, RBM15, RECQL4, REL, RET, RHOH, RICTOR, RMI2, RNF213, RNF43, ROS1, RPL10, RPL22, RPL5, RPN1, RPTOR, RUNX1, RUNx1T1, SBDS, SDC4, SDHAF2, SDHB, SDHC, SDHD, SEPT5, SEPT6, SEPT9, SET, SETBP1, SETD2, SF3B1, SFPQ, SH2B3, SH3GL1, SLC34A2, SLC45A3, SMAD2, SMAD4, SMARCA4, SMARCB1, SMARCE1, SMO, SNX29, SOCS1, SOX10, SOX2, SPECC1, SPEN, SPOP, SRC, SRGAP3, SRSF2, SRSF3, SS18, SS18L1, SSX1, STAG2, STAT3, STAT4, STATSB, STIL, STK11, SUFU, SUZ12, SYK, TAF15, TAL1, TAL2, TBL1XR1, TCEA1, TCF12, TCF3, TCF7L2, TCL1A, TERT, TET1, TET2, TFE3, TFEB, TFG, TFPT, TFRC, TGFBR2, THRAP3, TLX1, TLX3, TMPRSS2, TNFAIP3, TNFRSF14, TNFRSF17, TOP1, TP53, TPM3, TPM4, TPR, TRAF7, TRIM26, TRIM27, TRIM33, TRIP11, TRRAP, TSC1, TSC2, TSHR, TTL, U2AF1, UBR5, USP6, VEGFA, VEGFB, VHL, VTI1A, WAS, WHSC1, WHSC1L1, WIF1, WISP3, WRN, WT1, WWTR1, XPA, XPC, XPO1, YWHAE, ZBTB16, ZMYM2, ZNF217, ZNF331, ZNF384, ZNF521, ZNF703, ZRSR2.

In still another embodiment, nucleic acid sequence analysis is used to assess at least one gene, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 250, 300, 350, 400, 450, 500, 550, 600, 650, 700, or all genes, selected from the group consisting of 5T4, ABI1, ABL1, ABL2, ACKR3, ACSL3, ACSL6, ActRIIA, ACVR1B, ADGRA2, AFF1, AFF3, AFF4, AKAP9, AKT1, AKT2, AKT3, ALDH2, ALK, AMER1, ANG1/ANGPT1/TM7SF2, ANG2/ANGPT2NPS51, APC, AR, ARAF, ARFRP1, ARHGAP26, ARHGEF12, ARID1A, ARID1B, ARID2, ARNT, ASPSCR1, ASXL1, ATF1, ATIC, ATM, ATP1A1, ATP2B3, ATR, ATRX, AURKA, AURKB, AXIN1, AXL, BAP1, BARD1, BBC3, BCL10, BCL11A, BCL11B, BCL2, BCL2L1, BCL2L11, BCL2L2, BCL3, BCL6, BCL7A, BCL9, BCOR, BCORL1, BCR, BIRC3, BLM, BMPR1A, BR2, BRAF, BRCA1, BRCA2, BRD3, BRD4, BRIP1, BTG1, BTK, BUB1B, c-KIT, C11orf30, C15orf65, C2orf44, CACNA1D, CALR, CAMTA1, CANT1, CARD11, CARS, CASC5, CASP8, CBFA2T3, CBFB, CBL, CBLB, CBLC, CCDC6, CCNB1IP1, CCND1, CCND2, CCND3, CCNE1, CD110, CD123, CD137, CD137/4, CD19, CD22, CD274, CD27L, CD38, CD4, CD74, CD79A, CD79B, CDC73, CDH1, CDH11, CDK12, CDK4, CDK6, CDK7, CDK8, CDKN1A, CDKN1B, CDKN2A, CDKN2B, CDKN2C, CDX2, CEBPA, CHCHD7, CHD2, CHD4, CHEK1, CHEK2, CHIC2, Chk1, CHN1, CIC, CIITA, CLP1, CLTC, CLTCL1, CNBP, CNOT3, CNTRL, COL1A1, COPB1, CoREST, COX6C, CRAF, CREB1, CREB3L1, CREB3L2, CREBBP, CRKL, CRLF2, CRTC1, CRTC3, CSF1R, CSF3R, CTCF, CTLA4, CTNNA1, CTNNB1, CUL3, CXCR4, CYLD, CYP17A1, CYP2D6, DAXX, DDB2, DDIT3, DDR1, DDR2, DDX10, DDX5, DDX6, DEK, DICER1, DLL-4, DM4, DNAPK, DNM2, DNMT3A, DOT1L, DS6, EBF1, ECT2L, EGFR, EIF4A2, ELF4, ELK4, ELL, ELN, EML4, EP300, EPHA3, EPHA5, EPHA7, EPHA8, EPHB1, EPHB2, EPS15, ERBB2, ERBB3, ERBB4, ERC1, ERCC1, ERCC2, ERCC3, ERCC4, ERCC5, ERG, ERRFI1, ESR1, ETBR, ETV1, ETV4, ETV5, ETV6, EWSR1, EXT1, EXT2, EZH2, EZR, FAK, FAM46C, FANCA, FANCC, FANCD2, FANCE, FANCF, FANCG, FANCL, FAS, FAT1, FBXO11, FBXW7, FCRL4, FEV, FGF, FGF10, FGF14, FGF19, FGF23, FGF3, FGF4, FGF6, FGFR1, FGFR1OP, FGFR2, FGFR3, FGFR4, FH, FHIT, FIP1L1, FKBP12, FLCN, FLI1, FLT1, FLT3, FLT4, FNBP1, FOXA1, FOXL2, FOXO1, FOXO3, FOXO4, FOXP1, FRS2, FSTL3, FUBP1, FUS, GABRA6, GAS7, GATA1, GATA2, GATA3, GATA4, GATA6, GCC, GID4, GITR, GLI1, GMPS, GNA11, GNA13, GNAQ, GNAS, GNRH1, GOLGA5, GOPC, GPC3, GPHN, GRIN2A, GRM3, GSK3B, H3F3A, H3F3B, HCK, HERPUD1, HEY1, HGF, HIP1, HIST1H3B, HIST1H4I, HLF, HMGA1, HMGA2, HMGN2P46, HMT, HNF1A, HNRNPA2B1, HOOK3, HOXA11, HOXA13, HOXA9, HOXC11, HOXC13, HOXD11, HOXD13, HRAS, HSD3B1, HSP90AA1, HSP90AB1, IAP, IDH1, IDH2, IGF1R, IGF2, IKBKE, IKZF1, IL2, IL21R, IL6, IL6ST, IL7R, INHBA, INPP4B, IRF2, IRF4, IRS2, ITGAV, ITGB1, ITK, JAK1, JAK2, JAK3, JAZF1, JUN, KAT6A, KAT6B, KCNJ5, KDM5A, KDM5C, KDM6A, KDR, KDSR, KEAP1, KEL, KIAA1549, KIF5B, KIR3DL1, KLF4, KLHL6, KLK2, KMT2A, KMT2A (MLL), KMT2C, KMT2C (MLL3), KMT2D, KMT2D (MLL2), KRAS, KTN1, LASP1, LCK, LCP1, LGR5, LHFP, LIFR, LMO1, LMO2, LOXL2, LPP, LRIG3, LRP1B, LSD1, LYL1, LYN, LZTR1, MAF, MAFB, MAGI2, MALT1, MAML2, MAP2K1, MAP2K2, MAP2K4, MAP3K1, MAPK1, MAPK11, MAX, MCL1, MDM2, MDM4, MDS2, MECOM, MED12, MEF2B, MEK1, MEK2, MEN1, MET, MITF, MKL1, MLF1, MLH1, MLLT1, MLLT10, MLLT11, MLLT3, MLLT4, MLLT6, MMP9, MN1, MNX1, MPL, MPS1, MRE11A, MS4A1, MSH2, MSH6, MSI2, MSN, MST1R, MTCP1, MTOR, MUC1, MUC16, MUTYH, MYB, MYC, MYCL, MYCN, MYD88, MYH11, MYH9, NACA, NAE1, NBN, NCKIPSD, NCOA1, NCOA2, NCOA4, NDRG1, NF1, NF2, NFE2L2, NFIB, NFKB2, NFKBIA, NIN, NKX2-1, NONO, NOTCH1, NOTCH2, NOTCH3, NPM1, NR4A3, NRAS, NSD1, NT5C2, NTRK1, NTRK2, NTRK3, NUMA1, NUP214, NUP93, NUP98, NUTM1, NUTM2B, OLIG2, OMD, P2RY8, PAFAH1B2, PAK3, PALB2, PARK2, PARP1, PATZ1, PAX3, PAX5, PAX7, PAX8, PBRM1, PBX1, PCM1, PCSK7, PDCD1, PDCD1LG2, PDE4DIP, PDGFB, PDGFRA, PDGFRB, PDK1, PER1, PHF6, PHOX2B, PICALM, PIK3C2B, PIK3CA, PIK3CB, PIK3CD, PIK3CG, PIK3R1, PIK3R2, PIM1, PKC, PLAG1, PLCG2, PML, PMS1, PMS2, POLD1, POLE, POT1, POU2AF1, POU5F1, PPARG, PPP2R1A, PRCC, PRDM1, PRDM16, PREX2, PRF1, PRKAR1A, PRKCI, PRKDC, PRLR, PRRX1, PRSS8, PSIP1, PTCH1, PTEFb, PTEN, PTK2, PTPN11, PTPRC, PTPRD, QKI, RABEP1, RAC1, RAD21, RAD50, RAD51, RAD51B, RAF1, RALGDS, RANBP17, RANBP2, RANKL, RAP1GDS1, RARA, RB1, RBM10, RBM15, RECQL4, REL, RET, RHOH, RICTOR, RMI2, RNF213, RNF43, ROS1, RPL10, RPL22, RPL5, RPN1, RPS6KB1, RPTOR, RUNX1, RUNx1T1, SBDS, SDC4, SDHA, SDHAF2, SDHB, SDHC, SDHD, SEPT5, SEPT6, SEPT9, SET, SETBP1, SETD2, SF3B1, SFPQ, SH2B3, SH3GL1, SLAMF7, SLC34A2, SLC45A3, SLIT2, SMAD2, SMAD3, SMAD4, SMARCA4, SMARCB1, SMARCE1, SMO, SNCAIP, SNX29, SOCS1, SOX10, SOX2, SOX9, SPECC1, SPEN, SPOP, SPTA1, SRC, SRGAP3, SRSF2, SRSF3, SS18, SS18L1, SSX1, SSX2, SSX4, STAG2, STAT3, STAT4, STAT5B, STEAP1, STIL, STK11, SUFU, SUZ12, SYK, TAF1, TAF15, TAL1, TAL2, TBL1XR1, TBX3, TCEA1, TCF12, TCF3, TCF7L2, TCL1A, TERC, TERT, TET1, TET2, TFE3, TFEB, TFG, TFPT, TFRC, TGFB1, TGFBR2, THRAP3, TIE2, TLX1, TLX3, TMPRSS2, TNFAIP3, TNFRSF14, TNFRSF17, TOP1, TOP2A, TORK, TP53, TPM3, TPM4, TPR, TRAF7, TRIM26, TRIM27, TRIM33, TRIP11, TRRAP, TSC1, TSC2, TSHR, TTL, U2AF1, UAE, UBR5, USP6, VEGFA, VEGFB, VEGFR, VHL, VTI1A, WAS, WEE1, WHSC1, WHSC1L1, WIF1, WISP3, WNT, WRN, WT1, WWTR1, XPA, XPC, XPO1, YWHAE, ZAK, ZBTB16, ZBTB2, ZMYM2, ZNF217, ZNF331, ZNF384, ZNF521, ZNF703, and ZRSR2.

In an embodiment, nucleic acid sequence analysis is used to assess a copy number variation in at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or all of ABL1, AKT1, AKT2, ALK, ANG1/ANGPT1/TM7SF2, ANG2/ANGPT2/VPS51, APC, ARAF, ARID1A, ATM, AURKA, AURKB, BBC3, BCL2, BIRC3, BRAF, BRCA1, BRCA2, CCND1, CCND3, CCNE1, CDK4, CDK6, CDK8, CDKN2A, CHEK1, CHEK2, CREBBP, CRKL, CSF1R, CTLA4, CTNNB1, DDR2, EGFR, EP300, ERBB3, ERBB4, EZH2, FBXW7, FGF10, FGF3, FGF4, FGFR1, FGFR2, FGFR3, FLT3, GATA3, GNA11, GNAQ, GNAS, HNF1A, HRAS, IDH1, IDH2, JAK2, JAK3, KRAS, MCL1, MDM2, MLH1, MPL, MYC, NF1, NF2, NFKBIA, NOTCH1, NPM1, NRAS, NTRK1, PAX3, PAX5, PAX7, PAX8, PDGFRA, PDGFRB, PIK3CA, PTCH1, PTEN, PTPN11, RAF1, RB1, RET, RICTOR, ROS1, SMAD4, SRC, TOP1, TOP2A, TP53, VHL, and WT1.

In a further embodiment, nucleic acid sequence analysis is used to detect a gene fusion in at least 1, 2, 3, 4, 5, 6, 7, 8, 9, 10, 11, 12, 13, 14, 15, 16, 17, 18, 19, 20, 21, 22, 23, 24, 25, 26, 27, 28 or 29 of ALK, AR, BCR, BRAF, ETV1, ETV4, ETV5, ETV6, EWSR1, FGFR1, FGFR2, FGFR3, FUS, MYB, NFIB, NR4A3, NTRK1, NTRK2, NTRK3, PDGFRA, RAF1, RARA, RET, ROS1, SSX1, SSX2, SSX4, TFE3, and TMPRSS2. In a related embodiment, nucleic acid sequence analysis is used to detect a gene fusion in at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 of AKT3, ALK, ARHGAP26, AXL, BRAF, BRD3/4, EGFR, ERG, ESR1, ETV1/4/5/6, EWSR1, FGFR1/2/3, FGR, INSR, MAML2, MAST1/2, MET, MSMB, MUSK, MYB, NOTCH1/2, NRG1, NTRK1/2/3, NUMBL, NUTM1, PDGFRA/B, PIK3CA, PKN1, PPARG, PRKCA/B, RAF1, RELA, RET, ROS1, RSPO2/3, TERT, TFE3, TFEB, THADA and TMPRSS2.

The biological sample may be assessed using techniques which include, but are not limited to, IHC analysis, gene expression analysis, ISH analysis, and/or sequencing analysis (such as by PCR, RT-PCR, pyrosequencing, HTS, NGS) for at least one of the following: ABCC1, ABCG2, ACE2, ADA, ADH1C, ADH4, AGT, AR, AREG, ASNS, BCL2, BCRP, BDCA1, beta III tubulin, BIRC5, B-RAF, BRCA1, BRCA2, CA2, caveolin, CD20, CD25, CD33, CD52, CDA, CDKN2A, CDKN1A, CDKN1B, CDK2, CDW52, CES2, CK 14, CK 17, CK 5/6, c-KIT, c-Met, c-Myc, COX-2, Cyclin D1, DCK, DHFR, DNMT1, DNMT3A, DNMT3B, E-Cadherin, ECGF1, EGFR, EML4-ALK fusion, EPHA2, Epiregulin, ER, ERBR2, ERCC1, ERCC3, EREG, ESR1, FLT1, folate receptor, FOLR1, FOLR2, FSHB, FSHPRH1, FSHR, FYN, GART, GNA11, GNAQ, GNRH1, GNRHR1, GSTP1, HCK, HDAC1, hENT-1, Her2/Neu, HGF, HIF1A, HIG1, HSP90, HSP90AA1, HSPCA, IGF-1R, IGFRBP, IGFRBP3, IGFRBP4, IGFRBP5, IL13RA1, IL2RA, KDR, Ki67, KIT, K-RAS, LCK, LTB, Lymphotoxin Beta Receptor, LYN, MET, MGMT, MLH1, MMR, MRP1, MS4A1, MSH2, MSHS, Myc, NFKB1, NFKB2, NFKBIA, NRAS, ODC1, OGFR, p16, p21, p27, p53, p95, PARP-1, PDGFC, PDGFR, PDGFRA, PDGFRB, PGP, PGR, PI3K, POLA, POLA1, PPARG, PPARGC1, PR, PTEN, PTGS2, PTPN12, RAF1, RARA, ROS1, RRM1, RRM2, RRM2B, RXRB, RXRG, SIK2, SPARC, SRC, SSTR1, SSTR2, SSTR3, SSTR4, SSTR5, Survivin, TK1, TLE3, TNF, TOP1, TOP2A, TOP2B, TS, TUBB3, TXN, TXNRD1, TYMS, VDR, VEGF, VEGFA, VEGFC, VHL, YES1, and ZAP70.

Exemplary biomarker-drug association rules include without limitation: performing IHC on PD1 to determine likely benefit or lack of benefit from a PD-1 modulating therapy, PD-1 inhibitor, anti-PD-1 immunotherapy, anti-PD-1 monoclonal antibody, nivolumab, pidilizumab (CT-011, CureTech, LTD), pembrolizumab (lambrolizumab, MK-3475, Merck), a PD-1 antagonist, a PD-1 ligand soluble construct, and/or AMP-224 (Amplimmune); performing IHC on PD-L1 to determine likely benefit or lack of benefit from a PD-L1 modulating therapy, PD-L1 inhibitor, anti-PD-L1 immunotherapy, anti-PD-L1 monoclonal antibody, BMS-936559, MPDL3280A/RG7446, and/or MEDI4736 (MedImmune); performing IHC on RRM1 to determine likely benefit or lack of benefit from an antimetabolite and/or gemcitabine; performing IHC on TS to determine likely benefit or lack of benefit from a antimetabolite, fluorouracil, capecitabine, and/or pemetrexed; performing IHC on TOPO1 to determine likely benefit or lack of benefit from a TOPO1 inhibitor, irinotecan and/or topotecan; performing at least one of IHC on MGMT, pyrosequencing for MGMT promoter methylation, and sequencing on IDH1 to determine likely benefit or lack of benefit from an alkylating agent, temozolomide, and/or dacarbazine; performing IHC on AR to determine likely benefit or lack of benefit from an anti-androgen, bicalutamide, flutamide, abiraterone and/or enzalutamide; performing IHC on ER to determine likely benefit or lack of benefit from a hormonal agent, tamoxifen, fulvestrant, letrozole, and/or anastrozole; performing IHC on at least one of ER, PR and AR to determine likely benefit or lack of benefit from a hormonal agent, tamoxifen, toremifene, fulvestrant, letrozole, anastrozole, exemestane, megestrol acetate, leuprolide, goserelin, bicalutamide, flutamide, abiraterone, enzalutamide, triptorelin, abarelix, and/or degarelix; performing at least one of IHC on HER2 and ISH on HER2 to determine likely benefit or lack of benefit from a tyrosine kinase inhibitor and/or lapatinib, pertuzumab, and/or ado-trastuzumab emtansine (T-DM1); performing at least one of IHC on HER2, ISH on HER2, IHC on PTEN and sequencing on PIK3CA to determine likely benefit or lack of benefit from HER2 targeted therapy, and/or trastuzumab; performing at least one of ISH on TOP2A, ISH on HER2, IHC on TOP2A and IHC on PGP to determine likely benefit or lack of benefit from an anthracycline, doxorubicin, liposomal-doxorubicin, and/or epirubicin; performing sequencing on at least one of cKIT and PDGFRA to determine likely benefit or lack of benefit from a tyrosine kinase inhibitor and/or imatinib; performing at least one of ISH on ALK and ISH on ROS1 to determine likely benefit or lack of benefit from a tyrosine kinase inhibitor and/or crizotinib; performing at least one of IHC on ER or sequencing on PIK3CA to determine likely benefit or lack of benefit from an mTOR inhibitor, everolimus, and/or temsirolimus; performing sequencing on RET to determine likely benefit or lack of benefit from a tyrosine kinase inhibitor, and/or vandetanib; performing IHC on at least one of TLE3, TUBB3 and PGP to determine likely benefit or lack of benefit from a taxane, paclitaxel, and/or docetaxel; performing IHC on SPARC to determine likely benefit or lack of benefit from a taxane, and/or nab-paclitaxel; performing at least one of PCR and sequencing on BRAF to determine likely benefit or lack of benefit from a tyrosine kinase inhibitor, vemurafenib, dabrafenib, and/or trametinib; performing at least one of sequencing on KRAS, sequencing on BRAF, sequencing on NRAS, sequencing on PIK3CA and IHC on PTEN to determine likely benefit or lack of benefit from an EGFR-targeted antibody, cetuximab, and/or panitumumab; performing sequencing on EGFR to determine likely benefit or lack of benefit from an EGFR-targeted antibody, and/or cetuximab; performing at least one of sequencing on EGFR, sequencing on KRAS, ISH on cMET, sequencing on PIK3CA and IHC on PTEN to determine likely benefit or lack of benefit from a tyrosine kinase inhibitor, erlotinib, and/or gefitinib; performing sequencing on EGFR to determine likely benefit or lack of benefit from a tyrosine kinase inhibitor, and/or afatinib; performing sequencing on cKIT to determine likely benefit or lack of benefit from a tyrosine kinase inhibitor, and/or sunitinib; performing sequencing on at least one of BRCA1, BRCA2 and/or IHC on ERCC1 to determine likely benefit or lack of benefit from carboplatin, cisplatin, and/or oxaliplatin; performing ISH on ALK to determine likely benefit or lack of benefit from ceritinib; and performing ISH to detect 1p19q codeletion to determine likely benefit or lack of benefit from procarbazine, lomustine, and/or vincristine (PCV).

Additional biomarkers of interest, descriptions thereof, and rules associating the states of various biomarkers to predicted therapeutic/drug efficacies can be found in US Patent Publications US20100113299, published May 6, 2010; US20140222443, published Aug. 7, 2014; US20150307947, published Oct. 29, 2015; US20160186266, published Jun. 30, 2016; and US20150024952, published Jan. 22, 2015; U.S. Pat. No. 8,700,335, issued Apr. 15, 2014 and U.S. Pat. No. 8,768,629, issued Jul. 1, 2014; and Int'l Patent Publications WO2015116868, published Aug. 6, 2015, and WO2016141169, published Sep. 9, 2016; each of which patent publications is incorporated herein by reference in its entirety.

The methods, systems, apparatus and/or computer program of the invention can be used to analyze biological data in any relevant setting. The setting can be related to a disease, such as a neoplastic/proliferative disease, neurological disease, autoimmune disease, cardiovascular disease, or infectious disease. In a preferred embodiment, the disease comprises a cancer, such as an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumor (GIST), glioblastoma, head and neck squamous carcinoma, leukemia, liver hepatocellular carcinoma, low grade glioma, lung bronchioloalveolar carcinoma (BAC), non-small cell lung cancer (NSCLC), small cell lung cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma. The cancer may be an acute lymphoblastic leukemia; acute myeloid leukemia; adrenocortical carcinoma; AIDS-related cancer; AIDS-related lymphoma; anal cancer; appendix cancer; astrocytomas; atypical teratoid/rhabdoid tumor; basal cell carcinoma; bladder cancer; brain stem glioma; brain tumor, brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma; breast cancer; bronchial tumors; Burkitt lymphoma; cancer of unknown primary site (CUP); carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor.

Another example relationship the apparatus and/or database may be configured to determine includes a relationship between a particular therapeutic regime and a biological sample that has been processed so as to indicate the presence of a particular biomarker. Accordingly, the method 300 may include performing a therapeutic regime analysis to determine a relationship status for the relationship between a therapeutic regime and at least one of the patient status and the marker status, 306. In this regard, the apparatus and/or database may be configured to determine a positive interrelationship status between a particular therapeutic regime and a positive marker status (i.e., a particular therapeutic regime is shown to be statistically more effective for a particular condition and/or disease in response to determining that a particular biomarker is detected in a patient's biological sample). The apparatus and/or database may include computer-readable program code that includes instructions for comparing a particular therapeutic regime with a marker status and/or patient status. Accordingly, the apparatus and/or database may be configured to determine that patients having particular attributes may respond more positively or negatively to a particular therapeutic regime than those patients that did not possess the particular attribute. Likewise, the apparatus and/or database may be configured to determine that patients whose biological sample indicated the presence of a particular marker may respond more positively or negatively to a particular therapeutic regime than those patients whose biological sample indicated the particular marker was absent. The presence of a particular marker may include an expression level, or presence of a particular mutation or other characteristic of interest.

Additionally, the method 300 may include displaying at least one graphical user interface on a user interface (e.g., user interface 110, FIG. 1) in communication with the computing device, that includes a plurality of graphical user interface objects, 308, wherein each GUI object may be associated with any one of the patient data, the relationship, and/or relationship status, and wherein at least one GUI object includes an indicium corresponding to at least one of the patient data, the relationship, and/or relationship status, wherein at least one GUI object is associated with a target patient and includes indicia corresponding to any one of the patient data, the relationship, and/or relationship status of the target patient. For example, the apparatus may be configured to display a GUI on a monitor that includes a plurality of GUI objects. In some embodiments, the GUI may include a plurality of differing views that provide visualization for analyzing biological data. For example, the GUI may include a reference data view as shown in FIGS. 4A and 4B, a cohort view as shown in FIGS. 4C and 4D, a waterfall plot as shown in FIGS. 4E through 4H, a matrix view as shown in FIGS. 4I and 4J, a volcano plot for a specific drug as shown in FIG. 4K, a volcano plot for a specific biomarker as shown in FIG. 4L, a demography table as shown in FIG. M, a view of a sunburst plot for the plurality of biomarkers as shown in FIG. 4N and FIG. 4O, a view of a sunburst plot for the plurality of drugs as shown in FIGS. 4P and 4Q, and a patient view as shown in FIG. 4R. One of ordinary skill in the art may appreciate that these various GUIs displayed are merely exemplary and that other suitable GUIs for analyzing the biological sample are encompassed within the disclosure herein.

According to some embodiments, the method 300 may include displaying at least one GUI that includes a first GUI object configured to cause the display of a second GUI object upon the selection thereof. In some embodiments, the first GUI object may include information corresponding to patient data and the second GUI object may include information corresponding to patient data that is not ascertainable from the first GUI object. That is, the first GUI object may provide information corresponding to the patient data at a higher level, while the second GUI object may provide additional details corresponding to information presented by the first GUI object that is not presented by the first GUI object. For example, as shown in FIG. 4A, a GUI 400A may include a first GUI object 402A and a second GUI object 404A. In this regard, FIG. 4A illustrates a GUI where the first GUI object 402A has been selected thereby causing the display of the second GUI object 404A. The first GUI object 402A includes information of the various conditions and/or diseases of patients included in the reference data. Upon selection of a portion of the first GUI object 402A (i.e., the region of the pie chart associated with Breast Carcinoma), the apparatus may cause the GUI to display a second GUI object 404A (i.e., a Kaplan Meier Plot corresponding to Breast Carcinoma cases). Accordingly, the second GUI object 404A provides information, indicia, and/or the like not previously ascertainable from the display of the first GUI object 402A.

In some additional embodiments, the method 300 may include an additional step comprising assisting in providing patient care based on the one or more interrelationships displayed on the user interface. For example, assisting in providing the patient care comprises providing a diagnosis, providing a prognosis, selecting a recommended therapeutic regime, generating a hypothesis, and evaluating an efficiency of the therapeutic regime, based on the one or more interrelationships. Accordingly, an apparatus and/or database may be configured to display a graphical user interface that provides for a visual comparison between patient data corresponding to a biological sampling event(s), a biological processing event(s), therapeutic regime(s), marker status(es), and/or patient status(es) of a target patient and reference data corresponding to any one of the biological sampling event(s), biological processing event(s), therapeutic regime(s), marker status(es), and/or patient status(es) for the plurality of patients included in the reference data. As such, a clinician may manipulate the graphical user interface and the various GUI objects displayed thereby to visually compare a particular target patient against a set of reference patients based on shared patient attributes, therapy regime(s), marker status(s), and/or the like so as to increase the likelihood of a positive outcome and/or patient status. When so doing, the target patient data may not appear in the same GUI object as the reference patients. For example, the clinician may have a molecular profiling report for the target patient and compare results in the report against reference patients with desired attributes displayed in the various GUI objects.

In this regard, FIGS. 4A-4R illustrate exemplary graphical user interfaces according to various embodiments of the present disclosure. In particular, a GUI 400 may include a plurality of GUI objects 402. As shown in FIG. 4A, a GUI 400A may be configured to display information corresponding to reference data associated with at least a biological sampling event, a biological processing event, a therapeutic regime, a marker status, and/or a patient status. For example, the GUI 400A may include a first GUI object 402A (i.e., a pie chart illustrating the breakdown of the conditions and/or diseases of the patients included in the reference data). Additionally, the GUI 400A may include a second GUI object 404A that may be displayed upon detecting a selection of a portion of the first GUI object 402A. For example, a user may select a portion of the first GUI object 402A (e.g., the portion of the pie chart corresponding to the patients having breast carcinoma) that may cause an apparatus to display the second GUI object 404A (e.g., a pop-up overlay displaying a Kaplan Meier plot of those patients having breast carcinoma) within the graphical user interface 400.

FIG. 4B illustrates a zoomed view of the second or secondary GUI object 404A in FIG. 4A. As previously mentioned, the second GUI object 404A comprises a Kaplan Meier plot that includes a plurality of indicia that correspond to patient data associated with a particular patient. In some embodiments, the plurality of indicia includes various demarcations and/or regions having differing colors, patterns, shapes, and/or the like that correspond with patient data associated with at least a biological sampling event, a biological processing event, at least one therapeutic regime, a marker status, and/or at least one patient status. For example, as shown in FIG. 4B, the second GUI object 404A illustrates a survival analysis (i.e., a determination of percent survival over a period of time) performed for a particular condition and/or disease lineage using patient data identifying matched patients and unmatched patients. In some embodiments, matched patient data includes patients who received one or more therapeutic regime that provides a benefit in treating a condition and/or disease and unmatched patient data includes patients who received one or more therapeutic regime that provides a potential lack of benefit in treating the condition and/or disease. As illustrated in reference to FIGS. 4A-4B, the condition and/or disease lineage comprises breast carcinoma.

FIG. 4C illustrates another GUI 400B that includes a first or primary GUI object 402B, a secondary GUI object 404B, a tertiary GUI object 406B, and a quaternary GUI object 408B. The GUI 400B, in some embodiments, comprises a waterfall plot over which the plurality of GUI objects 402B, 404B, 406B, 408B may be overlaid. Although the GUI 400B illustrated in FIG. 4C illustrates a plurality of GUI objects 402B, 404B, 406B, 408B being nested with respect to one another to a fourth level, various embodiments may be configured to provide for any number of GUI objects that are nested, related, and/or ordered with respect to one another to any number of levels. As shown in FIG. 4C, each primary GUI object 402B (i.e., each vertical column) corresponds to a particular patient and includes a plurality of indicia that corresponds to data associated with at least a biological sampling event, a biological processing event, a therapeutic regime, a marker status, and/or a patient status.

Upon detecting the selection of any one of the primary or first GUI objects 402B, an apparatus may be configured to display a secondary GUI object 404B that includes an overlay displaying information corresponding to the patient data. For example, the secondary GUI object 404B includes data associated with a patient's case identification number, a patient's gender, a patient's age at diagnosis of the condition and/or disease, the condition and/or disease afflicting the patient, the severity of the condition and/or disease at the time of diagnosis, the method in which the biological sample from the patient was processed, and/or the date/time when the biological sample was processed. Additionally, the secondary GUI object 404B may include additional objects (i.e., hyperlinks) that, upon selection thereof, may cause the apparatus to display a tertiary GUI object 406B. For example, a tertiary GUI object 406B may include indicia that provides information corresponding to biomarker status(es) determined during a processing of the biological sample of the patient. In some example embodiments, each of the indicia may be user selectable and upon the selection of any one of the indicia included in the tertiary GUI object 406B, the apparatus may cause the GUI 400B to display a quaternary GUI object 408B (e.g., a Kaplan Meier plot illustrating the survivability of those patients based on the selected indicia corresponding to a particular biomarker).

FIG. 4D illustrates a zoomed view of the region A in FIG. 4C. As previously mentioned, each of the primary GUI objects 402B (i.e., each of the vertical columns) in FIG. 4C correspond with a particular patient. As shown in greater detail in FIG. 4D, each of the primary GUI objects 402B may include a plurality of indicia that corresponds with patient data associated with the particular patient. For example, the plurality of indicia may include various demarcations and/or regions having differing colors, patterns, shapes, and/or the like that correspond with patient data associated with at least a biological sampling event, a biological processing event, at least one therapeutic regime, a marker status, and/or at least one patient status. For example, as shown in FIG. 4D, the primary GUI object 402B may include a biological processing event indicia 410B that provides information corresponding to when a biological processing event occurred. In this regard, as shown in FIGS. 4C and 4D, each of the primary GUI objects 402B may be aligned with respect to one another based upon the biological processing event. That is, each of the primary GUI objects 402B are aligned with respect to one another such that the biological processing event indicia 410B of each of the primary GUI objects are aligned.

The primary GUI object 402B may further include a biological sampling event indicia 412B indicating when the biological sample was obtained from the particular patient. As shown in FIG. 4D, a biological sampling event indicia 412B may be shown as a blue line and/or region within the primary GUI object 402B. Additionally, the primary GUI object 402B may include additional indicia corresponding to a therapeutic regime. For example, each of the therapeutic regime indicia 414B, 416B, 418B corresponds with a particular therapeutic regime administered to the patient. In some embodiments, the therapeutic regime indicia 414B, 416B, 418B may be a region of the primary GUI object 402B that has a height corresponding to the length of time the therapeutic regime was administered. Further, each of the therapeutic regime indicia 414B, 416B, 418B may further provide a visual indication corresponding to a relationship status between the particular therapeutic regime and at least one marker status. For example, beneficial therapeutic regime indicia 414B may be a region of the primary GUI object 402B shaded in a green color, which may correspond with a positive relationship (e.g., increased efficacy and/or probabilities of positive outcomes) between the particular therapeutic regime and a particular biomarker present in the patient's biological sample. Accordingly, a non-beneficial therapeutic regime indicia 418B may be a region of the primary GUI object 402B shaded in a red color, which may correspond with a negative relationship (e.g., decreased efficacy, lower probabilities of positive outcomes, and/or the like) between the particular therapeutic regime and the particular biomarker present in the patient's biological sample. In another embodiment, the primary GUI object 402B may include an inconclusive therapeutic regime indicia 416B that is shown in FIG. 4D as a yellow shaded region. The inconclusive therapeutic regime indicia 416B may indicate that a positive or negative relationship between the particular therapeutic regime and the particular biomarker present in the patient's biological sample has not been established by the apparatus.

Additionally, the primary GUI object 402B may include indicia of a patient status. For example, the primary GUI object 402B may include a treatment-free indicia 420B illustrating a portion of time where no therapeutic regimes were administered to the patient. In this regard, the treatment-free indicia 420B may be portions of the primary GUI object 402B shaded in a grey color. Additionally or alternatively, an indicia of a patient status may include a death indicia 422B, which may correspond to the point in time where the patient died. For example, the death indicia 422B may include a bolded black line at the top of the primary GUI object 402B, which indicates the point in time when the patient died. As such, a clinician may visually compare patient data corresponding to a target patient against reference data for the plurality of patients by causing the apparatus to display a GUI object that corresponds to the target patient (i.e., causing the apparatus to display an additional column that includes indicia corresponding to patient data associated with the target patient). Additionally or alternatively, the apparatus may be configured to perform a therapeutic regime analysis and display a comparison between the target patient data and the reference data by causing the display of an additional GUI object that corresponds to the target patient information.

FIG. 4E illustrates another example GUI 400C that includes a first or primary GUI object 402C, a secondary GUI object 404C, a tertiary GUI object 406C, and a quaternary GUI object 408C. The GUI 400C, in some embodiments, comprises each of the plurality of GUI objects 402C, 404C, 406C, and 408C sized to fit within the GUI 400C, rather than being overlaid (e.g., GUI 400B, FIG. 4C) with respect to each other. Although the GUI 400C illustrates the plurality of GUI objects 402C, 404C, 406C, and 408C being sized to fit within the GUI 400C, various embodiments of the GUI 400C are configurable to provide for any number of GUI objects to fit within the GUI 400C, as well as to be nested, related, and/or ordered with respect to one another and/or any number of levels.

As shown in FIG. 4E, the GUI 400C includes data from a specific database. For example, the patient data displayed in the GUI 400C comprises patient data received from a selected database, i.e., Registry_v1_3. Such patient data may be mapped, processed, and/or stored in a manner to be described below in reference to FIG. 5. Additionally, the GUI 400C is configured to display the patient data that is filtered based on at least one biomarker status, a patient status, at least one therapeutic regime, a biological processing event, and/or a biological sampling event. As shown in FIG. 4E, a filter is applied to thereto so that only patient data associated with a specific condition and/or disease lineage (i.e., breast carcinoma) is displayed.

In FIG. 4E, the primary GUI object 402C comprises a waterfall plot, similar to the waterfall plot displayed in the GUI 400B in FIG. 4C, where each vertical column of the waterfall plot corresponds to a particular patient and includes a plurality of indicia that corresponds to data associated with at least a biological sampling event, a biological processing event, a therapeutic regime, a marker status, and/or a patient status.

The patient data provided in the primary GUI object 402C is configured to be further analyzed using the secondary GUI object 404C. The secondary GUI object 404C comprises a plurality of selectable indicia for further filtering of the patient data provided in the primary GUI object 402C. More particularly, the secondary GUI object 404C provides a visual display of the selectable indicia to allow a user to efficiently identify one or more particular patient or a patient cohort presenting the indicium(a) selected by the user. For example, the secondary GUI object 404C comprises a selection panel such as that illustrated in greater detail in FIG. 4F. Primary indicia 410C for selection may be displayed in the secondary GUI object 404C. For example, the primary indicia 410C is individually selectable by the clinician. Upon selection of a primary indicium 410C, a drop down menu comprising a plurality of secondary indicia 412C associated with the selected primary indicium 410C is displayed. For example, FIG. 4E illustrates a primary indicium 410C comprising a biomarker and secondary indicia 412C comprising one or more techniques for detecting a specific biomarker. In this example, the one or more techniques comprise fragment analysis, immunohistochemistry (IHC), in situ hybridization (ISH), and next-generation sequencing (NGS), as well as one or more specific biomarker detectable through each technique. Selection of the specific biomarker may result in manipulating the primary GUI object 402, as described below.

FIG. 4F illustrates a detailed view of the secondary GUI object 404C of FIG. 4E. More particularly, a detailed listing of each of the primary indicium 410C and their associated secondary indicia 412C is illustrated. For example, a biomarker primary indicium 410C in FIG. 4F comprises additional techniques including epidermal growth factor receptor (EGFR) H-score, O6-methylguanine-methyltransferase (MGMT) methylation, rearrangement, and sequencing. FIG. 4F also illustrates additional primary indicium and associated secondary indicium. For example, additional primary indicium 410C comprises cohort data, patient (clinical), biomarker result(s), treatment data, and case data. Secondary indicia associated with the cohort primary indicium includes condition and/or disease lineage(s), and match or unmatched. Secondary indicia associated with the patient (clinical) primary indicium includes age, sex, stage at diagnosis, and histology. Secondary indicia associated with the biomarker result primary indicium includes status of biomarker result (e.g., positive, negative, unknown, normal). Secondary indicia associated with the treatment primary indicium includes total drugs. Secondary indicia associated with the case primary indicium includes case list (e.g., a listing of de-identified patients). As may be apparent to one of ordinary skill in the art, the example primary indicium 410C and associated secondary indicia 412C described above are merely examples and are in no way limiting.

Exemplary indicia that can be used to filter the patient data displayed in the GUI objects are listed in Table 1. One of skill will appreciate that the filters can be updated as additional patient data having additional attributes is acquired.

TABLE 1
Filter indicia

| Indicium | Exemplary Values |
|---|---|
| Cancer Lineage | Breast Carcinoma, Non-small cell lung cancer (NSCLC), Ovarian Surface |
| | Epithelial Carcinoma, Gastroesophageal Adenocarcinoma, Soft Tissue Tumors, |
| | Cancer of Unknown Primary (CUP), Colorectal Adenocarcinoma, |
| | Neuroendocrine tumors, Female Genital Tract Malignancy, Leiomyosarcoma, |
| | Neuroblastoma, Pancreatic Adenocarcinoma, Lymphoma, Urinary Tract, |
| | Melanoma, Head and neck Squamous Carcinoma, Liver Hepatocellular |
| | Carcinoma, Cholangiocarcinoma, Lung Bronchioloalveolar carcinoma (BAC), |
| | Major & Minor Salivary Glands, Adrenal cortical carcinoma, Prostatic |
| | Adenocarcinoma, Glioblastoma, Non Epithelial Ovarian Cancer (non-EOC), |
| | Anal Cancer, Epithelial Skin Cancer, Paraganglioma, Small Intestinal |
| | Malignancies, Uveal Melanoma |
| Histology | Infiltrating duct carcinoma (NOS), Adenocarcinoma (NOS), Serous |
| | cystadenocarcinoma (NOS), Signet ring cell adenocarcinoma, Alveolar soft |
| | part sarcoma, Carcinoma (NOS), Intestinal type adenocarcinoma, |
| | Endometrioid adenocarcinoma (NOS), Papillary serous cystadenocarcinoma, |
| | Small cell carcinoma (NOS), Clear cell adenocarcinoma (NOS), Squamous cell |
| | carcinoma (NOS), Leiomyosarcoma (NOS), Mucinous adenocarcinoma, |
| | Neuroblastoma (NOS), Ewing sarcoma, Undifferentiated carcinoma (NOS), |
| | Mixed cell adenocarcinoma, Infiltrating ductular carcinoma, Non-small cell |
| | carcinoma, Carcinosarcoma (NOS), Epithelioid sarcoma, Diffuse large B-cell |
| | malignant lymphoma (NOS), Papillary transitional cell carcinoma, Amelanotic |
| | melanoma, Papillary carcinoma (NOS), Transitional cell carcinoma (NOS), |
| | Endocervical adenocarcinoma type, Mullerian mixed tumor, Desmoplastic |
| | small round cell tumor, Adenocarcinoma with mixed subtypes, Neuroendocrine |
| | carcinoma (NOS), Intraductal papillary adenocarcinoma with invasion, Serous |
| | adenocarcinofibroma, Adenosquamous carcinoma, Hepatocellular carcinoma |
| | (NOS), Large cell neuroendocrine carcinoma, Lobular carcinoma (NOS), |
| | Papillary adenocarcinoma (NOS), Cholangiocarcinoma, Invasive intraductal |
| | papillary-mucinous carcinoma, Keratinizing squamous cell carcinoma (NOS), |
| | Renal cell carcinoma (NOS), Malignant melanoma (NOS), Sarcoma (NOS), |
| | Mucinous bronchiolo-alveolar carcinoma, Tubular adenoma (NOS), Infiltrating |
| | lobular carcinoma (NOS), Ductal carcinoma (NOS), Clear cell |
| | adenocarcinofibroma, Mucinous cystadenocarcinoma (NOS), Large cell |
| | carcinoma (NOS), Metaplastic carcinoma (NOS), Serous surface papillary |
| | carcinoma, Papillary serous adenocarcinoma, Infiltrating duct adenocarcinoma, |
| | Infiltrating duct and lobular carcinoma, Duct adenocarcinoma (NOS), |
| | Collecting duct carcinoma, Adrenal cortical carcinoma, Spindle cell sarcoma, |
| | Gliosarcoma, Poorly differentiated Sertoli-Leydig cell tumor, Infiltrating |
| | lobular mixed with other types of carcinoma, Malignant teratoma (NOS), |
| | Nodular melanoma, Myxoid leiomyosarcoma, Adenocarcinoma in |
| | adenomatous polyp, Malignant mixed tumor (NOS), Mesodermal mixed tumor, |
| | Pheochromocytoma, malignant, Spindle cell squamous cell carcinoma, |
| | Papillary squamous cell carcinoma, Signet ring cell carcinoma, Glioblastoma |
| | (NOS), Malignant epithelioid hemangioendothelioma, Serous adenocarcinoma |
| | (NOS), Malignant chondroblastoma, Pleomorphic liposarcoma, Malignant |
| | peripheral nerve sheath tumor, Myxoid liposarcoma, Adenosarcoma, Eccrine |
| | adenocarcinoma, In situ infiltrating duct mixed with other types of carcinoma, |
| | Medullary carcinoma (NOS), Malignant granulosa cell tumor, Osteosarcoma |
| | (NOS), Malignant desmoplastic melanoma, Malignant fibrous histiocytoma, |
| | Malignant neoplasm, Liposarcoma (NOS), Serous carcinoma (NOS), Primary |
| | serous papillary carcinoma of peritoneum, Papillary urothelial carcinoma, |
| | Clear cell carcinoma |
| Stage | I, II, III, IV |
| Biomarkers | EGFR H-score: EGFR |
| | Fragment Analysis: ALK, microsatellite instability (MSI) |
| | IHC: Androgen Receptor, BCRP, c-KIT, CAV-1, CK, CK14, CK17, cMET, |
| | COX-2, Cyclin D1, ECAD, EGFR, ER, ERCC1, Her2, IGF1R, Ki67, MGMT, |
| | MLH1, MRP1, MSH2, MSH6, p53, PD-1, PD-L1, PDGFR, PGP, PMS2, PR, |
| | PTEN, RRM1, SPARC, TLE3, TOP2A, TOPO1, TS, TUBB3 |
| | ISH: cMET, cMYC, EGFR, Her2, PIK3CA, TOP2A |
| | Methylation: MGMT Promoter |
| | Rearrangement: ALK (2p23), ROS1 |
| | Sequencing: ABL1, AKT1, ALK, APC, ATM, BRAF, BRCA1, BRCA2, c- |
| | KIT, CDH1, cMET, CSF1R, CTNNB1, EGFR, ERBB2, ERBB4, FBXW7, |
| | FGFR1, FGFR2, FLT3, GNA11, GNAQ, GNAS, HNF1A, HRAS, IDH1, |
| | JAK2, JAK3, KDR, KRAS, MLH1, MPL, NOTCH1, NPM1, NRAS, |
| | PDGFRA, PIK3CA, PTEN, PTPN11, RB1, RET, SMAD4, SMARCB1, SMO, |
| | STK11, TP53, VHL |
| Drug | carboplatin, cisplatin, cyclophosphamide, doxorubicin hydrochloride, |
| | fluorouracil, gemcitabine hydrochloride, methotrexate, patupilone, vinorelbine |
| | tartrate, docetaxel, erlotinib hydrochloride, pemetrexed disodium, paclitaxel, |
| | pegylated liposomal doxorubicin hydrochloride, topotecan hydrochloride, |
| | epirubicin hydrochloride, trabectedin, bevacizumab, etoposide, irinotecan |
| | hydrochloride, nab-paclitaxel, trastuzumab, letrozole, capecitabine, leucovorin |
| | calcium, oxaliplatin, cetuximab, floxuridine, panitumumab, tamoxifen citrate, |
| | sorafenib tosylate, temozolomide, vincristine sulfate, ifosfamide, imatinib |
| | mesylate, imc-a12, temsirolimus, anastrozole, lapatinib ditosylate, |
| | investigational agent, prednisone, rituximab, everolimus, vinblastine sulfate, |
| | paclitaxel poliglumex, goserelin acetate, octreotide acetate, exemestane, |
| | fulvestrant, interferon, bortezomib, ziv-aflibercept, melphalan, cytarabine, |
| | hydrocortisone sodium succinate, pazopanib hydrochloride, raloxifene, |
| | investigational agent/placebo, 1-leucovorin, pralatrexate, megestrol acetate, |
| | bibf1120, dactinomycin, eribulin mesylate, mitomycin c, dasatinib, sunitinib |
| | malate, leuprolide acetate, bleomycin sulfate, mitoxantrone hydrochloride, |
| | dacarbazine, crizotinib, afatinib dimaleate, thalidomide, ado-trastuzumab |
| | emtansine, imiquimod, bicalutamide, ipilimumab, farletuzumab, abiraterone |
| | acetate, dalantercept, chlorambucil, degarelix, axitinib, recombinant interferon |
| | alfa-2a, lambrolizumab, cabozantinib-s-malate, veliparib, pertuzumab, |
| | regorafenib |
| Clinical | Age/s, Sex, Patient ID |
| Treatment | Matched, unmatched, mixed, indeterminate |
| Regimen | |

Referring back to FIG. 4E, the tertiary GUI object 406C includes a matrix of biomarkers and/or drugs associated with a particular patient displayed as a vertical column in the primary GUI object 402C (i.e., the waterfall plot). The tertiary GUI object 406C in FIG. 4E provides further indicia associated with each patient of the plurality of patients. More particularly, the tertiary GUI object 406C provides a distinct row associated with the selections made in the secondary GUI object 404C. Notably, manipulation of the secondary GUI object 404C results in manipulation of the tertiary GUI object 406C, as well as the primary GUI object 402C. For example, manipulating the secondary GUI object 404C includes selecting or deselecting, moving, adjusting, etc., indicia displayed in the GUI object. For example, selecting a secondary indicium 412C in the secondary GUI object 404C adds a corresponding row to the tertiary GUI object 406C, while unselecting a secondary indicium 412C in the secondary GUI object 404C removes the corresponding row from the tertiary GUI object 406C. For example, where a biomarker primary indicium 410C is selected and subsequent secondary indicia 412C of an Androgen Receptor, an Estrogen Receptor (ER), and a progesterone receptor (PR) are selected under the IHC technique, individual rows associated with each secondary indicium 412C are displayed in the tertiary GUI object 406C. In this example, the selection of ‘N’ or negative for each secondary indicium 412C in the secondary GUI object 404C results in visual indications in each added row for patients, e.g., where a ‘neg’ or negative value exists for each of the Androgen Receptor, the ER, and PR (thereby selecting for a patient cohort of triple negative breast cancer).

In some embodiments, patients exhibiting each of the secondary indicium 412C selected in the secondary GUI object 404C are grouped to one side of the tertiary GUI object 406C. Likewise, the primary GUI object 402C is modified based on the selection of primary and/or secondary indicium 410C, 412C in the secondary GUI object 404C. More particularly, the patient data provided in the primary GUI object 402C that comprises the indicia selected (i.e., ‘selected’ patients) in the secondary GUI object 404C may be manipulated such that this patient data is moved toward one side of the primary GUI object 402C and those cases that do not comprise the indicia selected (i.e., ‘unselected’ patients) in the secondary GUI object 404C may be manipulated such that this patient data is moved toward an opposite side of the primary GUI object 402C. For example and in reference to FIG. 4E, patients negative for the Androgen Receptor, ER, and PR (i.e., triple negative breast cancer) are all grouped to the left side of the GUI 400C in each of the primary GUI object 402C and the tertiary GUI object 406C, while patients who are not negative for all three of the Androgen Receptor, ER, and PR are all grouped to the right side of the GUI 400C in each of the primary GUI object 402C and the tertiary GUI object 406C.

Quaternary GUI object 408C provides one or more graphical comparisons of the patient data illustrated in the primary GUI object 402C based on the primary and secondary indicia 410C, 412C selected in the secondary GUI object 404C. For example, four Kaplan Meier plots with a survival analysis are provided as the quaternary GUI object 408C. The Kaplan Meier plots from left to right illustrate: i) a survival analysis comparing all patients who have been matched versus all patients who are unmatched; ii) a survival analysis comparing all selected patients versus all unselected patients; iii) a survival analysis comparing selected matched patients versus selected unmatched patients; and iv) a survival analysis comparing unselected matched patients versus unselected unmatched patients. However, the apparatus may be configured to process or analyze the data and display Kaplan Meier plots for indicia such as patient information, specific biomarkers, etc, as desired.

In another example embodiment, as shown in FIG. 4G, the quaternary GUI object 408C provides indicia associated with a specific patient selected in the primary GUI object 402C. For example, the quaternary GUI object 408C comprises graphical and visual information in a patient demographic GUI object 414C, a patient treatment history GUI object 416C, and a specific biomarker GUI object 418C. The patient demographic GUI object 414C comprises a table with a plurality of indicia specific to the patient selected (i.e., the vertical column selected) in the primary GUI object 402C. Such indicia may include a de-identified patient number, gender, age, race, a primary tumor site, condition and/or disease lineage, histology, followed (i.e., matched or unmatched), etc. The treatment history GUI object 416C comprises a graphical representation of specific drugs or therapeutic regimes and a length of treatment time for each drug or therapeutic regime. The effectiveness of each specific drug or therapeutic regime relative to the particular biomarker present may be indicated by color, shading, etc., or any other manner corresponding to the particular therapeutic regime and the particular biomarker present in the patient's biological sample displayed in the primary GUI object 402C.

The specific biomarker GUI object 418C comprises a list view of the technique used to assess the patient and the presence or absence of a specific biomarker resulting from that technique. The presence of a specific biomarker may be displayed differently than the absence of a specific biomarker. For example, the specific biomarker GUI object 418C comprises two different techniques, ISH and IHC. In this example, a Ribonucleotide Reductase Catalytic Subunit M1 (RRM1) was not detected (i.e., was absent) in the patient's biological sample using IHC. The RRM1 is, thus, colored in red in specific biomarker GUI object 418C. Further to this example, an Androgen Receptor was detected (i.e., was present) in the patient's biological sample using IHC. The Androgen Receptor is, thus, colored in green in the specific biomarker GUI object 418C.

In some embodiments, a quinary GUI object 420C is provided in the GUI 400C. The quinary GUI object 420C is, in some embodiments, nested or otherwise displayed relative to selection of indicia from the quaternary GUI object 408C. For example, as illustrated in FIG. 4G, the quaternary GUI object 420C is displayed upon selection of a specific biomarker in the specific biomarker GUI object 418C. Unlike the GUI objects 402C-408C, the quinary GUI object 420C overlays the GUI 400C, rather than being sized to fit within; though, in some embodiments, the quinary GUI object 420C is sized to fit within the GUI 400C. The quinary GUI object 420C is configured to provide additional data regarding a specific biomarker. For example, as illustrated in FIG. 4G, the quinary GUI object 420C is configured to display the biomarker name, the technique, the status of the biomarker, and/or an image corresponding to analysis of the biomarker. In this case, the image comprises a stained IHC slide, and other appropriate images may be displayed for different analysis techniques (e.g., traces for Sanger sequencing, fluorescence microscopy images for FISH, etc). Further selection of the quinary GUI object 420C results in providing a senary GUI object 422C including an image of the specific biomarker in greater detail in either overlay form or sized to fit within the GUI 400C.

In another example, as shown in FIG. 4H, the quaternary GUI object 408C provides indicia associated with a specific biomarker selected in the secondary GUI object 404C. For example, the quaternary GUI object 408C comprises graphical and visual information in a biomarker behavior GUI object 424C, a survival analysis GUI object 426C, and a statistic GUI object 428C. The biomarker behavior GUI object 424C comprises a volcano plot with an indication of a specific biomarker's behavior in various condition and/or disease lineages. For example, where a PR biomarker is selected in the secondary GUI object 404C, the behavior of that biomarker in condition and/or disease lineages such as breast carcinoma, ovarian surface epithelial carcinoma, etc., is displayed. Selecting a certain condition and/or disease lineage in the biomarker behavior GUI object 424C results in displaying a quinary GUI object 430C including additional information corresponding to patient data. For example, a Kaplan Meier plot for that condition and/or disease lineage is displayed by the quinary GUI object 430C. For example, selection of the breast carcinoma lineage in the quaternary GUI object 424C results in displaying a survival analysis Kaplan Meier plot 430C that overlies portions of the GUI objects 402C-408C.

The survival analysis GUI object 426C comprises a Kaplan Meier plot for a specific condition and/or disease lineage (e.g., ovarian surface epithelial carcinoma) where the specific biomarker is detected and where it is undetected. The statistic GUI object 428C provides a bar graph illustrating a ratio of positive and negative biomarker statuses for the specific biomarker selected in the secondary GUI object 404C for different condition and/or disease lineages. As desired, the data behind statistic GUI object 428C may be drawn from a larger cohort of patient data, e.g., including all patients that have been appropriate biomarker status data as opposed to only those with therapeutic regimen data such as in GUI object 402C.

In another example embodiment, as shown in FIGS. 4I and 4J, a graphical user interface 400D may include a matrix illustrating a marker status for each biomarker in the biological sample determined during a biological processing event. For example, a primary GUI object 402D may be defined by each cell of the matrix displayed by the GUI 400D. In this regard, FIG. 4J illustrates the region B of FIG. 4I in greater detail.

Further, each column of the matrix may correspond to an individual patient, while each row may correspond to a specific biomarker tested during the biological sampling event. Further, upon selection of the primary GUI object 402D, the apparatus may be configured to cause the display of a secondary GUI object 404D upon the graphical user interface 400D. In particular, selection of a primary GUI object 402D may cause the apparatus to display an overlay that includes additional information corresponding to the primary GUI object. For example, the secondary GUI object 404D may include information corresponding to the marker status, the biological processing event, and/or the like.

Additionally or alternatively, each of the primary GUI objects 402D may further include an indicium that corresponds with a relationship status between the marker status and the patient. For example, a primary GUI object 402D may include a particular color, shade, pattern, and/or the like within the matrix cell to indicate the presence of a particular biomarker in the biological sample taken for the particular patient. For example, as shown in FIG. 4J, a positive marker status indicium 430D corresponding to the presence and/or detection of a particular biomarker in the biological sample from the specific patient may include a matrix cell filled with a red color. Likewise a negative marker status indicium 432D corresponding to the absence of a particular biomarker in the biological sample from the particular patient may include a matrix cell filled with a blue color. Additional colors may be utilized to indicate an additional or alternative relationship status between the marker status and the patient.

In another embodiment, a graphical user interface 400F, 400G may be configured to display a volcano plot corresponding to a particular therapeutic regime (e.g., a drug) and/or a particular marker (e.g., a biomarker). In particular, FIG. 4K illustrates a volcano plot of a log-rank test p-value with respect to a hazard ratio for the selected therapeutic regime in different conditions and/or diseases (i.e., differing condition and/or disease lineages). Likewise, FIG. 4L illustrates a volcano plot of a log-rank test p-value with respect to a hazard ratio for the selected biomarker in different conditions and/or diseases. In this regard, FIG. 4K includes a primary GUI object 402F that corresponds with patient data associated with a particular condition (e.g., breast carcinoma). Upon selection of the primary GUI object 402F, a secondary GUI object 404F (i.e., an overlay) may be displayed by the GUI 400F that includes information corresponding to the efficacy of the selected therapeutic regime for the particular condition. Likewise, as shown in FIG. 4L, upon selection of the primary GUI object 402G displayed by the GUI 400G, the apparatus may cause the display of a secondary GUI object 404G that includes information corresponding to the presence of the selected biomarker for a particular condition.

In another embodiment, a GUI 400E is configured to display a demography table GUI object 402E. The demography table GUI object 402E is configured to display indicia relating to patient data for the plurality of patients. For example, the indicia comprise a specific condition and/or disease lineage, status of the condition and/or disease (e.g., stage), matched patients, unmatched patients, a match ratio, etc. In another embodiment, the GUI 400E is configured to display another demography table GUI object 404E. The demography table GUI object 404E is also configured to display indicia relating to patient data for the plurality of patients. In some embodiments, the indicia displayed in the demography table GUI object 402E and the indicia displayed in the demography table GUI object 404E differs based on a filter applied. For example, the filter is based on at least one biomarker status, the patient status, the at least one therapeutic regime, the biological processing event, and/or the biological sampling event. The filter applied to the patient data that is displayed in the demography table GUI object 402E comprises a stage (i.e., stage I, II, III, IIIA, IIIB, IIIC, IV, or unknown). The filter applied to the patient data that is displayed in the demography table GUI object 404E may comprise a specific biomarker and the technique used to assess the biomarker (i.e., PR IHC, ER IHC, etc).

The GUI 400E also displays a primary GUI object 406E as an overlay to the demography table on which the primary GUI object 406E is based. More particularly, the indicia provided in the demography table (e.g., 402E, 404E) is selectable by a clinician to be transformed into various types of graphical, textual, tabular, etc., displays. As illustrated in FIG. 4M, the indicia provided in the demography table GUI object 404E is used as the data for the primary GUI object 406E, which comprises a volcano plot. The volcano plot 406E in FIG. 4M is configured to display indicia corresponding to a particular assessment technique and/or a particular biomarker. In particular, the volcano plot GUI object 406E illustrates a volcano plot of a log-rank test p-value with respect to a hazard ratio for different biomarkers detected using the selected assessment technique (e.g., IHC). In this regard, upon selection of a specific biomarker within the volcano plot GUI object 406E, a secondary GUI object 408E (i.e., an overlay) may be displayed by the GUI 400E. The secondary GUI object 408E comprises a Kaplan Meier survival analysis for indicia based on the specific biomarker and assessment technique selected in the primary GUI object 406E.

According to yet another aspect, as shown in FIG. 4N, a graphical user interface 400H may be configured to provide a first or primary GUI object 402H that upon selection, may be configured to cause a secondary GUI object 404H to be displayed by the GUI 400H. In particular, FIG. 4N illustrates a sunburst plot detailing the relationship between any one of a marker status, a biological processing, and/or a patient's condition and/or disease. In this regard, a primary GUI object 402H, as shown in FIG. 4N, visually indicates the percentage of biological samples that include a particular biomarker (e.g., RRM1 in this example) that were obtained using IHC for conditions and/or diseases that are classified as “Others”.

Accordingly, each concentric circle and/or ring of the sunburst plots illustrated in FIGS. 4N-4Q represents a relationship between a particular level (i.e. a particular circle and/or ring) and preceding and/or subsequent levels. In this regard, FIGS. 4N and 4O illustrate the relationships between the biomarker, the condition and/or disease, and a particular processing method for detecting the biomarker. Likewise, FIGS. 4P and 4Q illustrate the relationships between a particular therapeutic regime and a patient condition and/or disease.

For example, referring to FIG. 4N, a top level GUI object 406H indicates the sunburst plot illustrates the relationship between the top level GUI object (e.g., biomarkers) and the subsequent level GUI objects. A secondary level GUI object 408H is defined by the portion of the sunburst plot demarcated by an angular interval. Accordingly, a relationship between the total number of patients and the particular condition and/or disease (e.g., breast carcinoma) is illustrated by the size of the angular interval and the corresponding portion defined thereby. A tertiary level GUI object 410H and a quaternary level GUI object 412H may illustrate the relationship between a method of processing a biological sample, a biomarker status, and/or a patient condition and/or disease. For example, the size of the tertiary level GUI object 410H, as defined by the portion of the tertiary ring demarcated by a particular angular interval may visually indicate the percentage of patients that have a particular condition (e.g., breast carcinoma) whose biological sample was processed using a particular method (e.g., immunohistochemistry). Likewise, the size of the quaternary level GUI object 412H may visually indicate the percentage of patients that have a particular condition (e.g., breast carcinoma) whose biological sample was processed using a particular method (e.g., immunohistochemistry) that have a positive indication for a particular biomarker (e.g., RRM1).

Additionally, in some embodiments, the selection of an intermediate level GUI object may cause the apparatus to display a GUI 400I that illustrates an intermediate level sunburst plot. For example, selection of the secondary level GUI object 408H in a GUI 400H of a top level sunburst plot, as shown in FIG. 4N, may cause the apparatus to display a GUI 400I of an intermediate level sunburst plot, as shown in FIG. 4O. Accordingly, the angular interval demarcating the portion of the secondary level GUI object 408H increases to 360 degrees indicating the displayed intermediate level sunburst plot is associated with methods used for processing the biological sample and biomarkers detected for only a particular patient condition and/or disease (e.g., breast carcinoma). In some embodiments, selection of a primary level GUI object 406H provided by a GUI 400I illustrating an intermediate level sunburst plot may cause the apparatus to display a GUI 400H illustrating the top level sunburst plot. In this regard, FIGS. 4P and 4Q illustrates various sunburst plot GUIs 400J, 400K that are provided by an apparatus according to example embodiments of the present disclosure and act in a similar fashion to the sunburst plot GUIs 400H, 400I, as shown in FIGS. 4N and 4O, respectively. More particularly, the sunburst plot GUIs 400J, 400K illustrated in FIGS. 4P and 4Q illustrate the relationship between a particular therapeutic regime and a patient condition and/or disease.

In yet another embodiment of the present invention, FIG. 4R illustrates an apparatus that is configured to display a GUI 400L that includes a first GUI object 402L and a second GUI object 404L. In particular, the GUI 400L may include GUI objects, information, and/or indicia corresponding to a particular patient such as, for example a target patient. In another embodiment, the GUI 400L may include GUI objects, information, and/or indicia corresponding to a patient included in the reference data. Upon selection of the first GUI object 402L, which includes indicia corresponding with a relationship status between a therapeutic regime and a biomarker status for example, the apparatus may be configured to cause the GUI 400L to display a second GUI object 404L that includes additional, more detailed information corresponding to therapeutic regime and/or patient status. Indicia corresponding with a relationship status may include a variety of shading, coloring, highlighting, bolding, and/or any other suitable visual and/or audible signals that provide an indication of the relationship status.

FIG. 5 illustrates a schematic of a data storage arrangement, generally designated 500. The data storage arrangement 500 is generically embodied by the database 90 described in reference to FIG. 2. More particularly, the data storage arrangement 500 is configured to store and/or provide access to an application to one or more of the user devices 100A, 100B, 100C via the network 80 (see, FIG. 2). The user devices 100A, 100B, 100C are depicted in FIG. 5 as a computing device 502 comprising a hardware processor and memory and a display in communication therewith. Such a computing device 502 is a special purpose computing device that is configured to improve the art of providing patient care. For example, providing patient care using the computing device 502 comprises assisting with diagnosis, providing a prognosis, selecting a recommended therapeutic regime, generating a hypothesis, and evaluating an efficiency of the therapeutic regime, based on the one or more interrelationships.

The computing device 502 is configured to receive patient data from one or more database. In various embodiments, the one or more database comprises a clinical database 504, a biomarker database 506, a knowledge database 508, and/or a cohort database 510 comprising a combination of the clinical database 504, the biomarker database 506, and the knowledge database 508. Each database 506-510 is configured to receive updated patient data by either requesting such data or by receiving updated data from another source (e.g., a third party source, a government source, etc.). The patient data is then configured to be mapped in order to organize the data by various characteristics, such as at least a biological sampling event, a biological processing event, a therapeutic regime, a marker status, and/or a patient status. The patient data can be mapped at a repository or the like, 512, or may be mapped in each of databases 506-510. As illustrated in FIG. 5, the data is mapped at repository 512 separate from the databases 506-510.

After the data is mapped, the mapped data may be transmitted to one or more external database relative to the computing device 502. For example, a first external database 514A is a database that comprises original data from one or more of the clinical database 504, the biomarker database 506, the knowledge database 508, and/or the cohort database 510 including protected health information (PHI) data, as well as the mapped data from the repository 512. In some embodiments, the first external database 514A comprises the capabilities for staging the data contained within. More particularly, the first external database 514A is configured to stage the original data and the mapped data via processing the data in an extract, transform, and load (ETL) process. The ETL process is performed with validations or rules applied to the data in order to reject some of the patient data that does not comprise correct/expected values in a given domain. In some embodiments, the first external database 514A is configured to transmit the original data and the mapped data to a relational database for the staging of the data to be performed.

In some embodiments, after the data is staged, the staged data may be combined with further mapped data from the data repository. In this embodiment, the combined data may be transformed to prepare all the data for querying and analysis, e.g., by a clinician using the computing device 502. The first external database 514A is configured to either perform the data transformation itself or transmit the data stored within to a relational database.

In some embodiments, after the data is transformed at least a portion of the data is sent to a second external database 514B, where the data is combined with further mapped data and processed to integrate the data sets, as well as to remove any PHI data. The portion of the data sent to the second external database 514B comprises data that is ready to be queried, searched, analyzed, etc., at the computing device 502. In some embodiments, all of the data is considered ‘load production ready’ data and is transmitted to the second external database 514B. As illustrated in FIG. 5, it is the second external database 514B that is in communication with the computing device 502. However, in some embodiments, not illustrated, the first external database 514A and the second external database 514B are the same and all back-end data processing is performed in the same database that is in communication with the computing device 502.

The patient data stored in at least the second external database 514B can be configured to have limited access. Accordingly, in some embodiments, one or more user defined roles are created in order to restrict specific users from viewing specific portions of the patient data and/or manipulating the mapped patient data stored. For example, the user defined roles enable a particular user to access only patient data from the clinical database 504, another user to access only patient data from the biomarker database 506, another user to access patient data from each of the clinical database, the biomarker database 506, and the knowledge database. In other examples, the user defined roles enable a particular user to only access data from the second external database 514B. The one or more user defined roles can be created based on any desired attribute, e.g., at least one of condition and/or disease lineage, a patient cohort, a user affiliation, or user's membership in a study group. As examples of each, user access may be restricted to view only patient data for a given lineage such as only breast cancer, a given patient cohort such as only patient data for patients having a certain biomarker profile or being treated at a certain hospital or physician practice group, or a study group such as only patients participating in a certain clinical trial or other study.

It will be understood that each block of the flowchart in FIG. 3, and combinations of blocks in the flowchart, may be implemented by various means, such as hardware and/or a computer program product comprising one or more computer-readable mediums having computer readable program instructions stored thereon. For example, one or more of the procedures described herein may be embodied by computer program instructions of a computer program product. In this regard, the computer program product(s) which may embody the procedures described herein may be stored by one or more memory devices of a mobile terminal, server, or other computing device and executed by a processor in the computing device. In some embodiments, the computer program instructions comprising the computer program product(s) which embody the procedures described above may be stored by memory devices of a plurality of computing devices. As will be appreciated, any such computer program product may be loaded onto a computer or other programmable apparatus to produce a machine, such that the computer program product including the instructions which execute on the computer or other programmable apparatus creates means for implementing the functions specified in the flowchart block(s). Further, the computer program product may comprise one or more computer-readable memories on which the computer program instructions may be stored such that the one or more computer-readable memories can direct a computer or other programmable apparatus to function in a particular manner, such that the computer program product comprises an article of manufacture which implements the function specified in the flowchart block(s). The computer program instructions of one or more computer program products may also be loaded onto a computer or other programmable apparatus to cause a series of operations to be performed on the computer or other programmable apparatus to produce a computer-implemented process such that the instructions which execute on the computer or other programmable apparatus implement the functions specified in the flowchart block(s). Accordingly, blocks of the flowchart support combinations of means for performing the specified functions. It will also be understood that one or more blocks of the flowchart, and combinations of blocks in the flowchart, may be implemented by special purpose hardware-based computer systems which perform the specified functions, or combinations of special purpose hardware and computer program product(s).

### EXAMPLES

### Example 1: Molecular Profiling Systems

Molecular profiling is performed to assist in determining a treatment regimen for a cancer. Using a molecular profiling approach, molecular characteristics of the disease itself are assessed to determine a candidate treatment. Thus, this approach provides the ability to select treatments without regard to the anatomical origin of the diseased tissue, or other traditional “one-size-fits-all” approaches that do not take into account personalized characteristics of a particular patient's affliction. The profiling comprises determining gene and gene product expression levels, gene copy number and mutation analysis. Treatments are identified that are of likely benefit or not against cancer cells that overexpress certain genes or gene products, underexpress certain genes or gene products, carry certain chromosomal aberrations or mutations in certain genes, or any other measurable molecular attributes as compared to reference cells. The system has the power to take advantage of any useful technique to measure any biological characteristic that can be linked to a therapeutic efficacy. The end result allows caregivers to expand the range of therapies available to treat patients, thereby providing the potential for longer life span and/or quality of life than traditional “one-size-fits-all” approaches to selecting treatment regimens.

This Example illustrates components of a molecular profiling system that performs analysis of a cancer sample using a variety of molecular assessment techniques that measure expression levels, chromosomal aberrations, mutations, rearrangements and other characteristics. The molecular “blueprint” of the cancer is used to generate a prioritized ranking of druggable targets and/or drug associated targets in tumor and their associated therapies. This Example provides components of a molecular profiling system that can be used to generate patient data for use in the methods, systems, storage medium and apparatus of the invention. Such components (e.g., biomarkers or analysis techniques) can be used as criterion to filter the visualized patient data as described herein.

Formalin-fixed paraffin-embedded (FFPE) tumor samples are received from a treating physician and are reviewed by a pathologist for quality control before subsequent analysis. Analysis methods and biomarkers assessed are as described in Tables 2-6. As indicated in Table 2, certain tests may be performed based on tumor lineage as desired. In Table 2, mutation and copy number variation (CNV) on DNA can be assessed using next-generation sequencing (NGS) of the biomarkers according to Tables 3 and 4, and fusions can be assessed using NGS on RNA on the biomarkers according to Table 5. Table 6 lists certain “hotspot” genes with mutations linked to drug efficacy, prognosis, and clinical trial enrollment. The tables generally refer to genes by their recognized gene names. Listing of gene names and descriptions can be found using a variety of online databases, including GeneCards® (www.genecards.org), HUGO Gene Nomenclature (www.genenames.org), Entrez Gene (www.ncbi.nlm.nih.gov/entrez/query.fcgi?db=gene), UniProtKB/Swiss-Prot (www.uniprot.org), UniProtKB/TrEMBL (www.uniprot.org), OMIM (www.ncbi.nlm.nih.gov/entrez/query.fcgi?db=OMIM), GeneLoc (genecards.weizmann.ac.il/geneloc/), and Ensembl (www.ensembl.org). Generally, gene symbols and names below correspond to those approved by HUGO, and protein names are those recommended by UniProtKB/Swiss-Prot. Where a protein name indicates a precursor, the mature protein is also implied. Gene and protein symbols may be used interchangeably herein and the meaning can be derived from context, e.g., NGS is used to analyze nucleic acids whereas IHC is used to analyze proteins.

TABLE 2
Molecular Profiling Tests

| | Immunohistochemistry (IHC) | Sequencing | Other |
|---|---|---|---|
| Tumor Lineage | Biomarkers | (NGS) | Biomarkers |
| Bladder | ERCC1, PD-L1, RRM1, TOP2A, | Mutation, CNV | |
| | TRKA/B/C (NTRK), TS, | Analysis (DNA) | |
| | TUBB3 | | |
| Breast | AR, ER, ERCC1, Her2/Neu, PD- | Mutation, CNV | TOP2A (CISH) |
| | L1, PR, PTEN, RRM1, TLE3, | Analysis (DNA) | |
| | TRKA/B/C | (NTRK) | |
| Cancer of Unknown | ERCC1, PD-L1, RRM1, TOPO1, | Mutation, CNV | |
| Primary (CUP) | TRKA/B/C (NTRK), TS, | Analysis (DNA) | |
| | TUBB3 | | |
| Cervix | ER, ERCC1, PD-L1, PR, RRM1, | Mutation, CNV | |
| | TOP2A, TOPO1, TRKA/B/C | Analysis (DNA) | |
| | (NTRK), TS, TUBB3 | | |
| Cholangiocarcinoma/ | ERCC1, Her2/Neu, PD-L1, | Mutation, CNV | |
| Hepatobillary | RRM1, TOPO1, TRKA/B/C | Analysis (DNA) | |
| | (NTRK), TS, TUBB3 | | |
| Colorectal | ERCC1, PD-L1, PTEN, TOPO1, | Mutation, CNV | MSI (Fragment |
| | TRKA/B/C (NTRK), TS | Analysis (DNA) | Analysis) |
| Endometrial | ER, ERCC1, PR, PD-L1, PTEN, | Mutation, CNV | MSI (Fragment |
| | RRM1, TOP2A, TOPO1, | Analysis (DNA) | Analysis) |
| | TRKA/B/C (NTRK), TS, | | |
| | TUBB3 | | |
| Gastric/Esophageal | ERCC1, Her2/Neu, PD-L1, | Mutation, CNV | |
| | TOP2A, TOPO1, TRKA/B/C | Analysis (DNA) | |
| | (NTRK), TS, TUBB3 | | |
| GIST | PD-L1, PTEN, TRKA/B/C | Mutation, CNV | |
| | (NTRK) ERCC1, PD-L1, TOPO1 | Analysis (DNA) | |
| Glioma | ERCC1, PD-L1, RRM1, | Mutation, CNV | MGMT |
| | TRKA/B/C (NTRK), TS, | Analysis (DNA); | Methylation |
| | TUBB3 | Fusion Analysis | (Pyro |
| | | (RNA) | Sequencing) |
| Head & Neck | ERCC1, PD-L1, RRM1, TOP2A, | Mutation, CNV | |
| | TRKA/B/C (NTRK), TUBB3 | Analysis (DNA) | |
| Kidney | ERCC1, MGMT, PD-L1, | Mutation, CNV | |
| | TRKA/B/C (NTRK), TUBB3 | Analysis (DNA) | |
| Melanoma | ERCC1, PD-L1, MGMT, | Mutation, CNV | |
| | TOP2A, TS | Analysis (DNA) | |
| Non-Small Cell Lung | ALK, ERCC1, PD-L1, PTEN, | Mutation, CNV | |
| | RRM1, TOPO1, TRKA/B/C | Analysis (DNA); | |
| | (NTRK), TS, TUBB3 | Fusion Analysis | |
| | | (RNA) | |
| Ovarian | ER, ERCC1, PD-L1, PR, RRM1, | Mutation, CNV | |
| | TOP2A, TOPO1, TRKA/B/C | Analysis (DNA) | |
| | (NTRK), TUBB3 | | |
| Pancreatic | ERCC1, MLH1, MSH2, MSH6, | Mutation, CNV | |
| | PD-L1, PMS2, RRM1, TOPO1, | Analysis (DNA) | |
| | TRKA/B/C (NTRK), TS, | | |
| | TUBB3 | | |
| Prostate | AR, ERCC1, PD-L1, TRKA/B/C | Mutation, CNV | |
| | (NTRK), TUBB3 | Analysis (DNA) | |
| Sarcoma | ERCC1 , MGMT, PD-L1, RRM1, | Mutation, CNV | |
| | TOP2A, TOPO1, TRKA/B/C | Analysis (DNA) | |
| | (NTRK), TUBB3 | | |
| Thyroid | ERCC1, PD-L1, TOP2A, | Mutation, CNV | |
| | TRKA/B/C (NTRK) | Analysis (DNA) | |
| Other Tumors | ERCC1, PD-L1, RRM1, TOP2A, | Mutation, CNV | |
| | TRKA/B/C (NTRK), TS, | Analysis (DNA) | |
| | TUBB3 | | |

TABLE 3
Next-Generation Sequencing Mutation Analysis
Mutations

ABI1
ABL1
ACKR3
AKT1
AMER1 (FAM123B)
AR
ARAF
ATP2B3
ATRX
BCL11B
BCL2
BCL2L2
BCOR
BCORL1
BRD3
BRD4
BTG1
BTK
C15orf65
CBLC
CD79B
CDH1
CDK12
CDKN2B
CDKN2C
CEBPA
CHCHD7
CNOT3
COL1A1
COX6C
CRLF2
DDB2
DDIT3
DNM2
DNMT3A
EIF4A2
ELF4
ELN
ERCC1
ETV4
FAM46C
FANCF
FEV
FOXL2
FOXO3
FOXO4
FSTL3
GATA1
GATA2
GNA11
GPC3
HEY1
HIST1H3B
HIST1H4I
HLF
HMGN2P46
HNF1A
HOXA11
HOXA13
HOXA9
HOXC11
HOXC13
HOXD11
HOXD13
HRAS
IKBKE
INHBA
IRS2
JUN
KAT6A (MYST3)
KAT6B
KCNJ5
KDM5C
KDM6A
KDSR
KLF4
KLK2
LASP1
LMO1
LMO2
MAFB
MAX
MECOM
MED12
MKL1
MLLT11
MN1
MPL
MSN
MTCP1
MUC1
MUTYH
MYCL (MYCL1)
NBN
NDRG1
NKX2-1
NONO
NOTCH1
NRAS
NUMA1
NUTM2B
OLIG2
OMD
P2RY8
PAFAH1B2
PAK3
PATZ1
PAX8
PDE4DIP
PHF6
PHOX2B
PIK3CG
PLAG1
PMS1
POU5F1
PPP2R1A
PRF1
PRKDC
RAD21
RECQL4
RHOH
RNF213
RPL10
SEPT5
SEPT6
SFPQ
SLC45A3
SMARCA4
SOCS1
SOX2
SPOP
SRC
SSX1
STAG2
TAL1
TAL2
TBL1XR1
TCEA1
TCL1A
TERT
TFE3
TFPT
THRAP3
TLX3
TMPRSS2
UBR5
VHL
WAS
ZBTB16
ZRSR2

TABLE 4
Next-Generation Sequencing Mutation and CNV Analysis
Mutations and Copy Number Variations (CNV)

| | | | | | | | | | |
|---|---|---|---|---|---|---|---|---|---|
| ABL2 | BRCA21 | COPB1 | ESR1 | FUS | KIT | MYB | PER1 | RUNX1 | TFG |
| ACSL3 | BRIP1 | CREB1 | ETV1 | GAS7 | KLHL6 | MYC | PICALM | RUNX1T1 | TFRC |
| ACSL6 | BUB1B | CREB3L1 | ETV5 | GATA3 | KMT2A | MYCN | PIK3CA | SBDS | TGFBR2 |
| | | | | | (MLL) | | | | |
| AFF1 | C11orf30 | CREB3L2 | ETV6 | GID4 | KMT2C | MYD88 | PIK3R1 | SDC4 | TLX1 |
| | (EMSY) | | | (C17orf39) | (MLL3) | | | | |
| AFF3 | C2orf44 | CREBBP | EWSR1 | GMPS | KMT2D | MYH11 | PIK3R2 | SDHAF2 | TNFAIP3 |
| | | | | | (MLL2) | | | | |
| AFF4 | CACNA1D | CRKL | EXT1 | GNA13 | KRAS | MYH9 | PIM1 | SDHB | TNFRSF14 |
| AKAP9 | CALR | CRTC1 | EXT2 | GNAQ | KTN1 | NACA | PML | SDHC | TNFRSF17 |
| AKT2 | CAMTA1 | CRTC3 | EZH2 | GNAS | LCK | NCKIPSD | PMS2 | SDHD | TOP1 |
| AKT3 | CANT1 | CSF1R | EZR | GOLGA5 | LCP1 | NCOA1 | POLE | SEPT9 | TP53 |
| ALDH2 | CARD11 | CSF3R | FANCA | GOPC | LGR5 | NCOA2 | POT1 | SET | TPM3 |
| ALK | CARS | CTCF | FANCC | GPHN | LHFP | NCOA4 | POU2AF1 | SETBP1 | TPM4 |
| APC | CASC5 | CTLA4 | FANCD2 | GPR124 | LIFR | NF1 | PPARG | SETD2 | TPR |
| ARFRP1 | CASP8 | CTNNA1 | FANCE | GRIN2A | LPP | NF2 | PRCC | SF3B1 | TRAF7 |
| ARHGAP26 | CBFA2T3 | CTNNB1 | FANCG | GSK3B | LRIG3 | NFE2L2 | PRDM1 | SH2B3 | TRIM26 |
| ARHGEF12 | CBFB | CYLD | FANCL | H3F3A | LRP1B | NFIB | PRDM16 | SH3GL1 | TRIM27 |
| ARID1A | CBL | CYP2D6 | FAS | H3F3B | LYL1 | NFKB2 | PRKAR1A | SLC34A2 | TRIM33 |
| ARID2 | CBLB | DAXX | FBXO11 | HERPUD1 | MAF | NFKBIA | PRRX1 | SMAD2 | TRIP11 |
| ARNT | CCDC6 | DDR2 | FBXW7 | HGF | MALT1 | NIN | PSIP1 | SMAD4 | TRRAP |
| ASPSCR1 | CCNB1IP1 | DDX10 | FCRL4 | HIP1 | MAML2 | NOTCH2 | PTCH1 | SMARCB1 | TSC1 |
| ASXL1 | CCND1 | DDX5 | FGF10 | HMGA1 | MAP2K1 | NPM1 | PTEN | SMARCE1 | TSC2 |
| ATF1 | CCND2 | DDX6 | FGF14 | HMGA2 | MAP2K2 | NR4A3 | PTPN11 | SMO | TSHR |
| ATIC | CCND3 | DEK | FGF19 | HNRNPA2B1 | MAP2K4 | NSD1 | PTPRC | SNX29 | TTL |
| ATM | CCNE1 | DICER1 | FGF23 | HOOK3 | MAP3K1 | NT5C2 | RABEP1 | SOX10 | U2AF1 |
| ATP1A1 | CD274 | DOT1L | FGF3 | HSP90AA1 | MCL1 | NTRK1 | RAC1 | SPECC1 | USP6 |
| | (PDL1) | | | | | | | | |
| ATR | CD74 | EBF1 | FGF4 | HSP90AB1 | MDM2 | NTRK2 | RAD50 | SPEN | VEGFA |
| AURKA | CD79A | ECT2L | FGF6 | IDH1 | MDM4 | NTRK3 | RAD51 | SRGAP3 | VEGFB |
| AURKB | CDC73 | EGFR | FGFR1 | IDH2 | MDS2 | NUP214 | RAD51B | SRSF2 | VTI1A |
| AXIN1 | CDH11 | ELK4 | FGFR1OP | IGF1R | MEF2B | NUP93 | RAF1 | SRSF3 | WHSC1 |
| AXL | CDK4 | ELL | FGFR2 | IKZF1 | MEN1 | NUP98 | RALGDS | SS18 | WHSC1L1 |
| BAP1 | CDK6 | EML4 | FGFR3 | IL2 | MET | NUTM1 | RANBP17 | SS18L1 | WIF1 |
| | | | | | (cMET) | | | | |
| BARD1 | CDK8 | EP300 | FGFR4 | IL21R | MITF | PALB2 | RAP1GDS1 | STAT3 | WISP3 |
| BCL10 | CDKN1B | EPHA3 | FH | IL6ST | MLF1 | PAX3 | RARA | STAT4 | WRN |
| BCL11A | CDKN2A | EPHA5 | FHIT | IL7R | MLH1 | PAX5 | RB1 | STAT5B | WT1 |
| BCL2L11 | CDX2 | EPHB1 | FIP1L1 | IRF4 | MLLT1 | PAX7 | RBM15 | STIL | WWTR1 |
| BCL3 | CHEK1 | EPS15 | FLCN | ITK | MLLT10 | PBRM1 | REL | STK11 | XPA |
| BCL6 | CHEK2 | ERBB2 | FLI1 | JAK1 | MLLT3 | PBX1 | RET | SUFU | XPC |
| | | (HER2) | | | | | | | |
| BCL7A | CHIC2 | ERBB3 | FLT1 | JAK2 | MLLT4 | PCM1 | RICTOR | SUZ12 | XPO1 |
| | | (HER3) | | | | | | | |
| BCL9 | CHN1 | ERBB4 | FLT3 | JAK3 | MLLT6 | PCSK7 | RMI2 | SYK | YWHAE |
| | | (HER4) | | | | | | | |
| BCR | CIC | ERC1 | FLT4 | JAZF1 | MNX1 | PDCD1 | RNF43 | TAF15 | ZMYM2 |
| | | | | | | (PD1) | | | |
| BIRC3 | CIITA | ERCC2 | FNBP1 | KDM5A | MRE11A | PDCD1LG2 | ROS1 | TCF12 | ZNF217 |
| | | | | | | (PDL2) | | | |
| BLM | CLP1 | ERCC3 | FOXA1 | KDR | MSH2 | PDGFB | RPL22 | TCF3 | ZNF331 |
| | | | | (VEGFR2) | | | | | |
| BMPR1A | CLTC | ERCC4 | FOXO1 | KEAP1 | MSH6 | PDGFRA | RPL5 | TCF7L2 | ZNF384 |
| BRAF | CLTCL1 | ERCC5 | FOXP1 | KIAA1549 | MSI2 | PDGFRB | RPN1 | TET1 | ZNF521 |
| BRCA11 | CNBP | ERG | FUBP1 | KIF5B | MTOR | PDK1 | RPTOR | TET2 | ZNF703 |

| | |
|---|---|
| CNTRL | TFEB |

TABLE 5
Next-Generation Fusions and Transcript Variants

| Gene Fusions (RNA) | | | | | | | | Variant Transcripts (RNA) | |
|---|---|---|---|---|---|---|---|---|---|
| ALK | BRAF | NTRK1 | NTRK2 | NTRK3 | RET | ROS1 | RSPO3 | EGFR vIII | MET Exon |
| | | | | | | | | | 14 Skipping |

TABLE 6
Next-Generation Sequencing Hotspots
Next-Generation Sequencing Hotspots

ABL1
AKT1
ALK
APC
ATM
BRCA1
BRCA2
BRAF
CDH1
CSF1R
CTNNB1
EGFR
ERBB2 (HER2)
ERBB4 (HER4)
FBXW7
FGFR1
FGFR2
FLT3
GNA11
GNAQ
GNAS
HNF1A
HRAS
IDH1
JAK2
JAK3
KDR (VEGFR2)
KIT (cKIT)
KRAS
MET (cMET)
MPL
NOTCH1
NPM1
NRAS
PDGFRA
PIK3CA
PTEN
PTPN11
RB1
RET
SMAD4
SMARCB1
SMO
STK11
TP53
VHL

The desired molecular tests from Tables 2-6 are performed and results analyzed. The results can be compared against a database of drug-biomarker associations to identify therapeutic drug regimens that are more or less likely to benefit the patient. Certain biomarker states may indicate that the patient is a candidate for enrollment in certain clinical trials. Exemplary biomarker-drug associations are shown in Table 7.

TABLE 7
Exemplary biomarker-drug associations

| Drug/Agent | Biomarker | Platform |
|---|---|---|
| aspirin | PIK3CA | NGS |
| (assoc. in CRC) | | |
| afatinib | EGFR | NGS |
| (assoc. in NSCLC) | ERBB2 (HER2) | NGS |
| afatinib + cetuximab | EGFR T790M | NGS |
| (combination assoc. | | |
| in NSCLC) | | |
| cabozantinib | cMET | NGS |
| (assoc. in NSCLC) | | |
| capecitabine, | TS | IHC |
| fluorouracil, | | |
| pemetrexed | | |
| carboplatin, | BRCA1 | NGS |
| cisplatin, oxaliplatin | BRCA2 | NGS |
| | ERCC1 | IHC |
| ceritinib | ALK | IHC |
| cetuximab, | BRAF | NGS |
| panitumumab | KRAS | NGS |
| (assoc. in CRC) | NRAS | NGS |
| | PIK3CA | NGS |
| | PTEN | IHC |
| cetuximab | EGFR | CISH |
| (assoc. in NSCLC) | | |
| crizotinib | ALK | IHC |
| | cMET | CISH, NGS |
| | ROS1 | FISH |
| dabrafenib, | BRAF | NGS |
| vemurafenib | | |
| dacarbazine, | MGMT | IHC |
| temozolomide | MGMT-Methylation | Pyrosequencing |
| | IDH1 (associated in High Grade Glioma) | NGS |
| docetaxel, paclitaxel, | TLE3 | IHC |
| nab-paclitaxel | TUBB3 | IHC |
| doxorubicin, | HER2/Neu | CISH |
| liposomal- | TOP2A | IHC |
| doxorubicin, | | CISH |
| epirubicin | | |
| erlotinib, gefitinib | EGFR | NGS |
| (assoc. in NSCLC) | KRAS | NGS |
| | PIK3CA | NGS |
| | cMET | CISH |
| | PTEN | IHC |
| everolimus, | ER (associated in Breast) | IHC |
| temsirolimus | PIK3CA | NGS |
| gemcitabine | RRM1 | IHC |
| hormone therapies | AR | IHC |
| | ER | IHC |
| | PR | IHC |
| imatinib | cKIT | NGS |
| | PDGFRA | NGS |
| irinotecan | TOPO1 | IHC |
| topotecan | | |
| (excluding Breast, | | |
| CRC, NSCLC) | | |
| lapatinib, | HER2/Neu | IHC; CISH |
| pertuzumab, T-DM1 | | |
| lomustine, | 1p19q | FISH |
| procarbazine, | | |
| vincristine | | |
| mitomycin-c | BRCA1 | NGS |
| | BRCA2 | |
| nivolumab, | PD-L1 | IHC |
| pembrolizumab | | |
| (assoc. in Bladder, | | |
| Kidney, Melanoma, | | |
| NSCLC) | | |
| olaparib | BRCA1 | NGS |
| (assoc. in Ovarian) | BRCA2 | |
| osimertinib | EGFR T790M | NGS |
| (assoc. in NSCLC) | | |
| palbociclib | ER | IHC |
| (assoc. in Breast) | HER2/Neu | IHC; CISH |
| sunitinib | cKIT | NGS |
| (assoc. in GIST) | | |
| trametinib | BRAF | NGS |
| (assoc. in Melanoma) | | |
| trastuzumab | ERBB2 (HER2) | NGS |
| | HER2/Neu | IHC; CISH |
| | PTEN (associated in Breast) | IHC |
| | PIK3CA (associated in Breast) | NGS |
| vandetanib | RET | NGS |
| clinical trials | EGFR PTEN | IHC |
| clinical trials | EGFRvIII | Fragment Analysis |
| clinical trials | cMET | CISH; NGS |
| clinical trials | MLH1, MSH2, MSH6, | PMS2 IHC |
| | MSI | Fragment Analysis |
| clinical trials | ABL1, AKT1, ALK, APC, ATM, CSF1R, | NGS |
| | CTNNB1, EGFR, ERBB2 (Her2), FGFR1, FGFR2, | |
| | FLT3, GNA11, GNAQ, GNAS, HRAS, IDH1, | |
| | JAK2, KDR (VEGFR2), KRAS, MPL, NOTCH1, | |
| | NRAS, PTEN, SMO, TP53, VHL | |

In reference to Table 7, cetuximab/panitumumab, vemurafenib/dabrafenib, and trametinib may be reported in combination in colorectal cancer (CRC). Hormone therapies may include tamoxifen, toremifene, fulvestrant, letrozole, anastrozole, exemestane, megestrol acetate, leuprolide, goserelin, bicalutamide, flutamide, abiraterone, enzalutamide, triptorelin, abarelix, and degarelix. Abbreviations in Table 7 are as used herein: CRC: colorectal cancer; NSCLC: non-small cell lung cancer; IHC: Immunohistochemistry; CISH: Chromogenic in situ Hybridization; FISH: Fluorescence in situ Hybridization; NGS: Next-Generation Sequencing.

A report is generated from the above molecular profiling system. The report contains listings of drugs that are more likely to benefit the patient, less likely to benefit the patient, and of indeterminate benefit. The report is used by a treating physician to assist in providing a treatment plan for the patient whose tumor was profiled. Ultimate treatment decisions lie with the treating physician.

Further details of systems and methods for molecular profiling, including without limitation listings of biomarkers and biomarker-drug association rules, and exemplary molecular profiling reports, can be found in US Patent Publications US20100113299, published May 6, 2010; US20140222443, published Aug. 7, 2014; US20150307947, published Oct. 29, 2015; US20160186266, published Jun. 30, 2016; and US20150024952, published Jan. 22, 2015; U.S. Pat. No. 8,700,335, issued Apr. 15, 2014 and U.S. Pat. No. 8,768,629, issued Jul. 1, 2014; and Int'l Patent Publications WO2015116868, published Aug. 6, 2015, and WO2016141169, published Sep. 9, 2016; each of which patent publications is incorporated herein by reference in its entirety.

### Example 2: Treatment Planning

A database is assembled comprising patient data from over 100,000 molecular profiles collected over several years. Outcomes data is available for over 5 years for 10% of the profiles.

An oncologist having a patient with triple negative breast cancer orders molecular profiling of a tumor sample collected from the patient during surgery. The oncologist receives a molecular profiling report. The oncologist queries the database using the visualization methods and apparatus of the invention through a secure web interface to examine treatments and outcomes for other patients with triple negative breast cancer. In one instance, the oncologist identifies previous triple negative breast cancer patients in a waterfall plot that have certain similar molecular profiling results as the current patient. Based on a combination of the molecular profiling report, the visualization analysis, and expert medical opinion, the oncologist selects a treatment regimen most likely to benefit the patient.

### Example 3: Hypothesis Generation

A database is assembled comprising patient data from over 100,000 molecular profiles collected over several years. Outcomes data is available for over 5 years for 10% of the profiles. A researcher is interested in identifying biomarker targets for triple negative breast cancer patients. The researcher queries the database using the visualization methods and apparatus of the invention through a secure web interface to examine treatments and outcomes for patients with triple negative breast cancer. The researcher identifies classes of drugs and biological pathways that correspond to the biological states and/or treatment of prior triple negative breast cancer patients. The researcher chooses to examine members of a biological pathway with a high incidence of mutations as candidates for development of targeted drug therapy.

Many modifications and other embodiments of the inventions set forth herein will come to mind to one skilled in the art to which these inventions pertain having the benefit of the teachings presented in the foregoing descriptions and the associated drawings. Therefore, it is to be understood that the embodiments of the invention are not to be limited to the specific embodiments disclosed and that modifications and other embodiments are intended to be included within the scope of the invention. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the invention. In this regard, for example, different combinations of elements and/or functions than those explicitly described above are also contemplated within the scope of the invention. Although specific terms are employed herein, they are used in a generic and descriptive sense only and not for purposes of limitation.

That which is claimed is:

1. A method of analyzing biological data, the method comprising:
receiving, at a computing device comprising a processor and memory, patient data for a plurality of patients, the patient data corresponding to at least one of a biological sampling event, a biological processing event, at least one therapeutic regime, at least one biomarker status, and a patient status;
determining at least one interrelationship between any one of the biological sampling event, the biological processing event, the at least one therapeutic regime, the at least one biomarker status, and the patient status;
performing a therapeutic regime analysis to determine an interrelationship status for the interrelationship between at least one therapeutic regime and at least one of the patient status and the at least one biomarker status; and
displaying at least one graphical interface on a user interface in communication with the computing device, the graphical interface including a plurality of visual elements, each visual element of the plurality of visual elements being associated with the patient data, at least one visual element being associated with the at least one interrelationship, at least one visual element including an indicium corresponding to at least one of the interrelationship status and the biomarker status.

2. The method according to claim 1, further comprising manipulating a primary visual element to display a secondary visual element including additional information corresponding to the patient data upon selection thereof.

3. The method according to claim 2, further comprising displaying the secondary visual element such that the secondary visual element overlays the primary visual element or the primary visual element is resized such that the secondary visual element is displayed adjacent to the primary visual element.

4. The method according to claim 1, further comprising assisting in providing patient care based on the one or more interrelationships displayed on the user interface.

5. The method according to claim 4, wherein assisting in providing the patient care comprises assisting in at least one of providing a diagnosis, providing a prognosis, selecting a recommended therapeutic regime, generating a hypothesis, and evaluating an efficiency of the therapeutic regime, based on the one or more interrelationships.

6. The method according to claim 4, wherein assisting in providing the patient care comprises selectively manipulating the graphical interface and one or more of the plurality of visual elements displayed thereon to visually compare a target patient against a set of reference patients.

7. The method according to claim 6, wherein visually comparing the target patient against the set of reference patients is based on shared patient attributes, the at least one therapeutic regime, and/or the at least one biomarker status.

8. The method according to claim 1, wherein performing the therapeutic regime analysis comprises identifying a positive interrelationship status between the at least one therapeutic regime and at least one positive biomarker status in response to determining that the at least one therapeutic regime is likely to be more effective for a condition and/or disease when a positive biomarker status for a particular biomarker is detected in the at least one biological sampling event.

9. The method according to claim 8, wherein the particular biomarker is a biomarker listed in any one of Tables 1-7.

10. The method according to claim 1, further comprising storing the patient data for the plurality of patients in a clinical database, a biomarker database, a knowledge database, and/or a cohort database comprising a combination of the clinical database, the biomarker database, and the knowledge database.

11. The method according to claim 10, further comprising mapping the patient data from the clinical database, the biomarker database, the knowledge database, and/or the cohort database and storing it in one or more external databases in communication with the computing device.

12. The method according to claim 11, further comprising creating one or more user defined roles to restrict specific users from viewing specific portions of the patient data and/or manipulating the mapped patient data stored in the one or more external databases.

13. The method according to claim 12, wherein the one or more user defined roles are based on at least one of disease lineage, patient cohort, user affiliation, or user's membership in a study group.

14. The method according to claim 1, wherein the plurality of visual elements comprises at least one of a sunburst plot, a Kaplan Meier plot, a waterfall plot, a table, a volcano plot, or a graph.

15. The method according to claim 1, further comprising applying a filter to the patient data to filter the patient data based on at least one of a particular biomarker, the at least one biomarker status, a patient cohort, a patient status, the at least one therapeutic regime, the biological processing event, the biological sampling event, at least one indicium listed in Table 1, and any combination thereof.

16. The method according to claim 15, wherein display of at least one of the plurality of visual elements is associated with the filtered patient data.

17. The method according to claim 1, wherein the patient data further comprises historical data that tracks the patient status over a period of time.

18. The method according to claim 17, wherein the patient status comprises information associated with an age of the patient, a sex of the patient, a race of the patient, a condition and/or disease of the patient, a status of the condition and/or disease of the patient, and/or an outcome of the condition and/or disease of the patient.

19. The method according to claim 18, wherein the outcome of the condition and/or disease of the patient comprises death, partial remission, complete remission, recurrence, or cure.

20. The method of claim 18, wherein the condition or disease of the patient comprises a cancer.

21. The method according to claim 20, wherein the lineage of the cancer is a lineage listed in Table 1.

22. The method according to claim 20, wherein the stage of the cancer comprises a stage listed in Table 1.

23. The method according to claim 20, wherein the histology of the cancer comprises a histology listed in Table 1.

24. The method according to claim 1, wherein determining the at least one interrelationship comprises determining an existence of a relationship between the patient status and the at least one biomarker status based on the biological sampling event.

25. The method according to claim 1, further comprising determining the at least one biomarker status by detecting at least one biomarker in the at least one biological sampling event and assessing at least one characteristic for the at least one particular biomarker, wherein the at least one characteristic comprises at least one of overexpression, underexpression, a modification, a polymorphism, a deletion, an insertion, a substitution, a translocation, a fusion, a break, a duplication, an amplification, a repeat, a copy number variant, a DNA methylation variation, a transcript expression level, a transcript variant, and a splice variant.

26. The method according to claim 25, wherein detecting the at least one particular biomarker in the at least one biological sampling event comprises assessing a biological sample from a patient using at least one assessment technique, the at least one assessment technique comprising gene expression analysis, nucleic acid sequence analysis, nucleic acid methylation analysis and/or proteomic analysis.

27. The method according to claim 26, wherein the at least one particular biomarker comprises a protein, a nucleic acid, a lipid, a carbohydrate, or any combination thereof.

28. The method according to claim 26 or 27, wherein the at least one particular biomarker is a biomarker listed in any one of Tables 1-7.

29. The method according to claim 1, further comprising processing the patient data to determine which members of the plurality of patients are matched and which members of the plurality of patients are unmatched.

30. The method according to claim 29, wherein matched patient data includes patients who received one or more therapeutic regime predicted to provide a benefit in treating a condition and/or disease and wherein unmatched patient data includes patients who received one or more therapeutic regime predicted to provide a potential lack of benefit in treating the condition and/or disease.

31. The method according to claim 30, further comprising performing a survival analysis with the unmatched and matched patient data.

32. The method according to claim 31, further comprising displaying on the at least one graphical user interface a visual element associated with the survival analysis.

33. The method according to claim 32, wherein the visual element associated with the survival analysis is a Kaplan Meier plot.

34. A computer-readable storage medium that is non-transitory and has computer-readable program code portions stored therein that, in response to execution by a processor, cause an apparatus to at least:
receive, at a computing device comprising the processor and memory, patient data for a plurality of patients, the patient data corresponding to at least one of a biological sampling event, a biological processing event, at least one therapeutic regime, at least one biomarker status, and a patient status;
determine at least one interrelationship between any one of the biological sampling event, the biological processing event, the at least one therapeutic regime, the at least one biomarker status, and the patient status;
perform a therapeutic regime analysis to determine an interrelationship status for the interrelationship between at least one therapeutic regime and at least one of the patient status and the at least one biomarker status; and
display at least one graphical interface on a user interface in communication with the computing device, the graphical interface including a plurality of visual elements, each visual element of the plurality of visual elements being associated with the patient data, at least one visual element being associated with the at least one interrelationship, at least one visual element including an indicium corresponding to at least one of the interrelationship status and the biomarker status.

35. The computer readable storage medium according to claim 34, wherein the apparatus is caused to manipulate a primary visual element to display a secondary visual element including additional information corresponding to the patient data upon selection thereof.

36. The computer readable storage medium according to claim 35, wherein the apparatus is caused to display the secondary visual element such that the secondary visual element overlays the primary visual element or the primary visual element is resized such that the secondary visual element is displayed adjacent to the primary visual element.

37. The computer readable storage medium according to claim 34, wherein the apparatus is caused to assist in providing patient care based on the one or more interrelationships displayed on the user interface.

38. The computer readable storage medium according to claim 37, wherein the apparatus is caused to assist in at least one of providing a diagnosis, providing a prognosis, selecting a recommended therapeutic regime, generating a hypothesis, and evaluating an efficiency of the therapeutic regime, based on the one or more interrelationships in order to assist in providing the patient care.

39. The computer readable storage medium according to claim 37, wherein the apparatus is caused to selectively manipulate the graphical interface and one or more of the plurality of visual elements displayed thereon to visually compare a target patient against a set of reference patients in order to assist in providing the patient care.

40. The computer readable storage medium according to claim 39, wherein a visual comparison of the target patient against the set of reference patients is based on shared patient attributes, the at least one therapeutic regime, and/or the at least one biomarker status.

41. The computer readable storage medium according to claim 34, wherein performing the therapeutic regime analysis comprises identifying a positive interrelationship status between the at least one therapeutic regime and at least one positive biomarker status in response to determining that the at least one therapeutic regime is likely to be more effective for a condition and/or disease when a positive biomarker status for a particular biomarker is detected in the at least one biological sampling event.

42. The computer readable storage medium according to claim 41, wherein the particular biomarker comprises a biomarker listed in any one of Tables 1-7.

43. The computer readable storage medium according to claim 34, wherein the apparatus is caused to store the patient data for the plurality of patients in a clinical database, a biomarker database, a knowledge database, and/or a cohort database comprising a combination of the clinical database, the biomarker database, and the knowledge database.

44. The computer readable storage medium according to claim 43, wherein the apparatus is caused to map the patient data from the clinical database, the biomarker database, the knowledge database, and/or the cohort database and storing it in one or more external databases in communication with the computing device.

45. The computer readable storage medium according to claim 44, wherein the apparatus is caused to create one or more user defined roles to restrict specific users from viewing specific portions of the patient data and/or manipulating the mapped patient data stored in the one or more external databases.

46. The computer readable storage medium according to claim 45, wherein the one or more user defined roles are based on at least one of disease lineage, patient cohort, user affiliation, or user's membership in a study group.

47. The computer readable storage medium according to claim 34, wherein the plurality of visual elements comprises at least one of a sunburst plot, a Kaplan Meier plot, a waterfall plot, a table, a volcano plot, or a graph.

48. The computer readable storage medium according to claim 34, wherein the apparatus is caused to apply a filter to the patient data to filter the patient data based on at least one of a particular biomarker, the at least one biomarker status, a patient cohort, a patient status, the at least one therapeutic regime, the biological processing event, the biological sampling event, at least one indicium listed in Table 1, and any combination thereof.

49. The computer readable storage medium according to claim 48, wherein display of at least one of the plurality of visual elements is associated with the filtered patient data.

50. The computer readable storage medium according to claim 34, wherein the patient data further comprises historical data that tracks the patient status over a period of time.

51. The computer readable storage medium according to claim 50, wherein the patient status comprises information associated with an age of the patient, a sex of the patient, a race of the patient, a condition and/or disease of the patient, a status of the condition and/or disease of the patient, and/or an outcome of the condition and/or disease of the patient.

52. The computer readable storage medium according to claim 51, wherein the outcome of the condition and/or disease of the patient comprises death, partial remission, complete remission, recurrence, or cure.

53. The computer readable storage medium according to claim 51, wherein the condition or disease of the patient comprises a cancer.

54. The computer readable storage medium according to claim 53, wherein the lineage of the cancer is a lineage listed in Table 1.

55. The computer readable storage medium according to claim 53, wherein the stage of the cancer comprises a stage listed in Table 1.

56. The computer readable storage medium according to claim 53, wherein the histology of the cancer comprises a histology listed in Table 1.

57. The computer readable storage medium according to claim 34, wherein the apparatus is caused to determine an existence of a relationship between the patient status and the at least one biomarker status based on the biological sampling event to determine the at least one interrelationship.

58. The computer readable storage medium according to claim 34, wherein the apparatus is caused to determine the at least one biomarker status by detecting at least one biomarker in the at least one biological sampling event and assessing at least one characteristic for the at least one particular biomarker, wherein the at least one characteristic comprises at least one of overexpression, underexpression, a modification, a polymorphism, a deletion, an insertion, a substitution, a translocation, a fusion, a break, a duplication, an amplification, a repeat, a copy number variant, a DNA methylation variation, a transcript expression level, a transcript variant, and a splice variant.

59. The computer readable storage medium according to claim 58, wherein the apparatus is caused to assess a biological sample from a patient using data generated via at least one assessment technique, the at least one assessment technique comprising gene expression analysis, nucleic acid sequence analysis, nucleic acid methylation analysis and/or proteomic analysis to detect the at least one particular biomarker in the at least one biological sampling event

60. The computer readable storage medium according to claim 59, wherein the at least one particular biomarker comprises a protein, a nucleic acid, a lipid, a carbohydrate, or any combination thereof.

61. The computer readable storage medium according to claim 59 or 60, wherein the at least one particular biomarker is a biomarker listed in any one of Tables 1-7.

62. The computer readable storage medium according to claim 34, wherein the apparatus is caused to process the patient data to determine which members of the plurality of patients are matched and which members of the plurality of patients are unmatched.

63. The computer readable storage medium according to claim 62, wherein matched patient data includes patients who received one or more therapeutic regime predicted to provide a benefit in treating a condition and/or disease and wherein unmatched patient data includes patients who received one or more therapeutic regime predicted to provide a potential lack of benefit in treating the condition and/or disease.

64. The computer readable storage medium according to claim 63, wherein the apparatus is caused to perform a survival analysis with the unmatched and matched patient data.

65. The computer readable storage medium according to claim 64, wherein the apparatus is caused to display on the at least one graphical user interface a visual element associated with the survival analysis.

66. The computer readable storage medium according to claim 65, wherein the visual element associated with the survival analysis is a Kaplan Meier plot.

67. An apparatus for analyzing biological data, the apparatus including a user interface, and a computing device in communication with the user interface, the computing device comprising a processor and memory including computer-readable program code stored therein, the computer-readable code configured, upon the execution thereof by the processor, to cause the apparatus to:
receive patient data for a plurality of patients, the patient data corresponding to at least one of a biological sampling event, a biological processing event, at least one therapeutic regime, at least one biomarker status, and a patient status;
determine at least one interrelationship between any one of the biological sampling event, the biological processing event, the at least one therapeutic regime, the at least one biomarker status, and the patient status;
perform a therapeutic regime analysis to determine an interrelationship status for the interrelationship between at least one therapeutic regime and at least one of the patient status and the at least one biomarker status; and
display at least one graphical interface on the user interface, the graphical interface including a plurality of visual elements, each visual element of the plurality of visual elements being associated with the patient data, at least one visual element being associated with the at least one interrelationship, at least one visual element including an indicium corresponding to at least one of the interrelationship status and the biomarker status.

68. The apparatus according to claim 67, wherein the apparatus is caused to manipulate a primary visual element to display a secondary visual element including additional information corresponding to the patient data upon selection thereof.

69. The apparatus according to claim 67, wherein the apparatus is caused to display the secondary visual element such that the secondary visual element overlays the primary visual element or the primary visual element is resized such that the secondary visual element is displayed adjacent to the primary visual element.

70. The apparatus according to claim 67, wherein the apparatus is caused to assist in providing patient care based on the one or more interrelationships displayed on the user interface.

71. The apparatus according to claim 70, wherein the apparatus is caused to assist in at least one of providing a diagnosis, providing a prognosis, selecting a recommended therapeutic regime, generating a hypothesis, and evaluating an efficiency of the therapeutic regime, based on the one or more interrelationships in order to assist in providing the patient care.

72. The apparatus according to claim 70, wherein the apparatus is caused to selectively manipulate the graphical interface and one or more of the plurality of visual elements displayed thereon to visually compare a target patient against a set of reference patients in order to assist in providing the patient care.

73. The apparatus according to claim 72, wherein a visual comparison of the target patient against the set of reference patients is based on shared patient attributes, the at least one therapeutic regime, and/or the at least one biomarker status.

74. The apparatus according to claim 67, wherein performing the therapeutic regime analysis comprises identifying a positive interrelationship status between the at least one therapeutic regime and at least one positive biomarker status in response to determining that the at least one therapeutic regime is likely to be more effective for a condition and/or disease when a positive biomarker status for a particular biomarker is detected in the at least one biological sampling event.

75. The apparatus according to claim 74, wherein the particular biomarker is a biomarker listed in any one of Tables 1-7.

76. The apparatus according to claim 67, wherein the apparatus is caused to store the patient data for the plurality of patients in a clinical database, a biomarker database, a knowledge database, and/or a cohort database comprising a combination of the clinical database, the biomarker database, and the knowledge database.

77. The apparatus according to claim 76, wherein the apparatus is caused to map the patient data from the clinical database, the biomarker database, the knowledge database, and/or the cohort database and storing it in one or more external databases in communication with the computing device.

78. The apparatus according to claim 77, wherein the apparatus is caused to create one or more user defined roles to restrict specific users from viewing specific portions of the patient data and/or manipulating the mapped patient data stored in the one or more external databases.

79. The apparatus according to claim 78, wherein the one or more user defined roles are based on at least one of disease lineage, patient cohort, user affiliation, or user's membership in a study group.

80. The apparatus according to claim 67, wherein the plurality of visual elements comprises at least one of a sunburst plot, a Kaplan Meier plot, a waterfall plot, a table, a volcano plot, or a graph.

81. The apparatus according to claim 67, wherein the apparatus is caused to apply a filter to the patient data to filter the patient data based on at least one of a particular biomarker, the at least one biomarker status, a patient cohort, a patient status, the at least one therapeutic regime, the biological processing event, the biological sampling event, at least one indicium listed in Table 1, and any combination thereof.

82. The apparatus according to claim 81, wherein display of at least one of the plurality of visual elements is associated with the filtered patient data.

83. The apparatus according to claim 67, wherein the patient data further comprises historical data that tracks the patient status over a period of time.

84. The apparatus according to claim 83, wherein the patient status comprises information associated with an age of the patient, a sex of the patient, a race of the patient, a condition and/or disease of the patient, a status of the condition and/or disease of the patient, and/or an outcome of the condition and/or disease of the patient.

85. The apparatus according to claim 84, wherein the outcome of the condition and/or disease of the patient comprises death, partial remission, complete remission, recurrence, or cure.

86. The apparatus according to claim 84, wherein the condition or disease of the patient comprises a cancer.

87. The apparatus according to claim 86, wherein the lineage of the cancer is a lineage listed in Table 1.

88. The apparatus according to claim 86, wherein the stage of the cancer comprises a stage listed in Table 1.

89. The apparatus according to claim 86, wherein the histology of the cancer comprises a histology listed in Table 1.

90. The apparatus according to claim 67, wherein the apparatus is caused to determine an existence of a relationship between the patient status and the at least one biomarker status based on the biological sampling event to determine the at least one interrelationship.

91. The apparatus according to claim 67, wherein the apparatus is caused to determine the at least one biomarker status by detecting at least one biomarker in the at least one biological sampling event and assessing at least one characteristic for the at least one particular biomarker, wherein the at least one characteristic comprises at least one of overexpression, underexpression, a modification, a polymorphism, a deletion, an insertion, a substitution, a translocation, a fusion, a break, a duplication, an amplification, a repeat, a copy number variant, a DNA methylation variation, a transcript expression level, a transcript variant, and a splice variant.

92. The apparatus according to claim 91, wherein the apparatus is caused to assess a biological sample from a patient using at least one assessment technique, the at least one assessment technique comprising gene expression analysis, nucleic acid sequence analysis, nucleic acid methylation analysis and/or proteomic analysis to detect the at least one particular biomarker in the at least one biological sampling event.

93. The apparatus according to claim 92, wherein the at least one biomarker comprises a protein, a nucleic acid, a lipid, a carbohydrate, or any combination thereof.

94. The apparatus according to claim 92 or 93, wherein the at least one particular biomarker is a biomarker listed in any one of Tables 1-7.

95. The apparatus according to claim 67, wherein the apparatus is caused to process the patient data to determine which members of the plurality of patients are matched and which members of the plurality of patients are unmatched.

96. The apparatus according to claim 95, wherein matched patient data includes patients who received one or more therapeutic regime predicted to provide a benefit in treating a condition and/or disease and wherein unmatched patient data includes patients who received one or more therapeutic regime predicted to provide a potential lack of benefit in treating the condition and/or disease.

97. The apparatus according to claim 96, wherein the apparatus is caused to perform a survival analysis with the unmatched and matched patient data.

98. The apparatus according to claim 97, wherein the apparatus is caused to display on the at least one graphical user interface a visual element associated with the survival analysis.

99. The apparatus according to claim 98, wherein the visual element associated with the survival analysis is a Kaplan Meier plot.

100. A method of analyzing biological data associated with a biological sample from a target patient, the method comprising:
receiving, at a computing device comprising a processor and memory, patient data associated with the target patient, the patient data corresponding to a biological sampling event, a biological processing event, a therapeutic regime, a marker status, and a patient status;
receiving reference data associated with a plurality of patients, the reference data corresponding to a plurality of biological sampling events, biological processing events, therapeutic regimes, marker statuses, and patient statuses;
determining at least one interrelationship between any one of the biological sampling events, the biological processing events, the therapeutic regimes, the marker statuses, and the patient statuses;
performing a therapeutic regime analysis to determine the interrelationship between at least one therapeutic regime and at least one of the at least one patient status and the at least one marker status;
displaying at least one graphical user interface, the graphical user interface configured to:
display a plurality of graphical user interface objects associated with the reference data,
display a plurality of graphical user interface objects associated with the patient data,
display, on at least one graphical interface on a user interface in communication with the computing device, a primary graphical user interface object configured to, upon receiving an indication of a user input defining a selection of the primary graphical user interface object, cause the graphical user interface to display a secondary graphical user interface object; and
assisting in providing patient care based on the one or more interrelationships displayed on the user interface.
